(19) United States
(12) Patent Application Publication
(10) Pub. No.: US 2013/0202614 A1
(43) Pub. Date: Aug. 8, 2013

(54) AUTOTAXIN PATHWAY MODULATION AND USES THEREOF

(71) Applicant: B.S.R.C. "Alexander Fleming", Varkiza (GR)

(72) Inventor: Vassilios Aidinis, Nea Smyrni (GR)

(73) Assignee: B.S.R.C. "ALEXANDER FLEMING", Varkiza (GR)

(21) Appl. No.: 13/693,329

(22) Filed: Dec. 4, 2012

Related U.S. Application Data
(63) Continuation of application No. PCT/EP2011/059224, filed on Jun. 3, 2011.
(60) Provisional application No. 61/351,681, filed on Jun. 4, 2010.

(51) Int. Cl. A61K 39/395; A61K 38/46; C12Q 1/34

(52) U.S. Cl. CPC A61K 39/3955; C12Q 1/34; A61K 38/465
USPC 424/158.1; 435/7.92; 506/9; 435/6.12; 424/94.6

(57) ABSTRACT

Disclosed are methods for preventing, treating, or reducing symptoms of a disorder involving the autotaxin (ATX) pathway. In one embodiment, the method features administering to a mammal a sufficient amount of an autotaxin (ATX) or lysophosphatidic acid (LPA) signaling inhibitor, to prevent, treat or reduce symptoms of an inflammatory disorder, autoimmune disorder, fibrosis or malignancy of the lung. Further disclosed are methods for diagnosing an autotaxin-related disorder as well as kits for performing the methods of the invention.

### CROSS-REFERENCE TO RELATED APPLICATIONS

This application is a continuation of International Application Serial No. PCT/EP2011/059224 filed Jun. 3, 2011, which itself claims priority to U.S. provisional application Ser. No. 61/351,681 filed Jun. 4, 2010, both of which are incorporated by reference herein in their entireties.

### FIELD OF THE INVENTION

The invention relates to methods for preventing, treating, or reducing systems of a disorder involving the autotaxin (ATX) pathway such as an inflammatory disease, autoimmune disorder, fibrosis or malignancy of the lung and to methods for diagnosing said diseases. Also disclosed are kits for performing the methods of the invention.

### BACKGROUND

Autotaxin (ATX, ENPP2) is a secreted glycoprotein widely present in biological fluids, including blood, cancer ascites, synovial, pleural and cerebrospinal fluids, originally isolated from the supernatant of melanoma cells as an autocrine motility stimulation factor^{1,2}. ATX is encoded by a single gene on human chromosome 8 (mouse chromosome 15) whose transcription, regulated by diverse transcription factors (Hoxa 13, NFAT-1 and v-jun), results in three alternatively spliced isoforms (α, β and γ)^{3,4}. ATX is synthesized as a prepro-enzyme, secreted into the extracellular space after the proteolytic removal of its N-terminal signal peptide⁵. ATX is a member of the ectonucleotide pyrophosphatase/phosphodiesterase family of ectoenzymes (E-NPP) that hydrolyze phosphodiesterase (PDE) bonds of various nucleotides and derivatives⁶. The enzymatic activity of ATX was enigmatic, until it was shown to be identical to lysophospholipase D (lysoPLD)⁷, which is widely present in biological fluids. Since ATX is a constitutively active enzyme, the biological outcome of ATX action will largely depend on its expression levels and the local availability of its substrates. The major lysophospholipid substrate for ATX, lysophosphatidylcholine (LPC), is secreted by the liver and is abundantly present in plasma (at about 100 μM) as a predominantly albumin bound form⁸. LPC is also detected in tumour-cell conditioned media 7, presumably as a constituent of shed microvesicles. ATX, through its lysoPLD activity converts lysophosphatidylcholine (LPC) to lysophosphatidic acid (LPA).

LPC is an important inflammatory mediator with recognized effects in multiple cell types and pathophysiological processes. It is a major component of oxidized low density lipoprotein (oxLDL) and it can exist in several other forms including free, micellar, bound to hydrophobic proteins such as albumin and incorporated in plasma membranes. It is produced by the hydrolysis of phosphatidylcholine (PC) by PLA2 with concurrent release of arachidonic acid and in turn of other pro-inflammatory mediators (prostaglandins and leukotrienes). Moreover, LPC externalization constitutes a chemotactic signal to phagocytic cells, while interaction with its receptors can also stimulate lymphocytic responses. LPC has been shown to have therapeutic effects in experimental sepsis, possibly by suppressing endotoxin-induced HMGB1 release from macrophages/monocytes.

LPA is a bioactive phospholipid with diverse functions in almost every mammalian cell line⁹. LPA is a major constituent of serum bound tightly to albumin, gelsolin and possibly other as yet unidentified proteins^{10,11}. LPA is also found in other biofluids, such as saliva and follicular fluid, and has been implicated in a wide array of functions, such as wound heeling, tumour invasion and metastasis, neurogenesis, myelination, astrocytes outgrowth and neurite retraction. The long list of LPA functions was also explained with the discovery that it signals through G-protein coupled receptors (GPCRs), via classical second messenger pathways. Five mammalian cell-surface LPA receptors have been identified so far. The best known are LPA₁₋₃ (namely Edg-2, Edg-4 and Edg7) which are all members of the so-called ‘endothelial differentiation gene’ (EDG) family of GPCRs¹². LPA receptors can couple to at least three distinct G proteins (G_{q}, Gᵢ and G_{12/13}), which, in turn, feed into multiple effector systems. LPA activates G_{q} and thereby stimulates Phospholipase C (PLC), with subsequent phosphatidylinositol-bisphosphate hydrolysis and generation of multiple second messengers leading to protein kinase C activation and changes in cytosolic calcium. LPA also activates Gᵢ, which leads to at least three distinct signaling routes: inhibition of adenylyl cyclase with inhibition of cyclic AMP accumulation; stimulation of the mitogenic RAS-MAPK (mitogen-activated protein kinase) cascade; and activation of phosphatidylinositol 3-kinase (PI3K), leading to activation of the guanosine diphosphate/guanosine triphosphate (GDP/GTP) exchange factor TIAM1 and the downstream RAC GTPase, as well as to activation of the AKT/PKB antiapoptotic pathway. Finally, LPA activates G_{12/13}, leading to activation of the small GTPase RhoA, which drives cytoskeletal contraction and cell rounding. So, LPA not only signals via classic second messengers such as calcium, diacylglycerol and cAMP, but it also activates RAS- and RHO-family GTPases, the master switches that control cell proliferation, migration and morphogenesis.

Previous studies have demonstrated that exogenous administration of lysophosphatidic acid (LPA) had significant anti-inflammatory activities in animal models of sepsis, by reducing the organ injury and increasing the mice survival to endotoxemia. 50% of circulating LPA is produced by the phospholipase D activity of Autotaxin (ATX) and the hydrolysis of lysophosphatidylcholine (LPC).

### SUMMARY OF THE INVENTION

As mentioned, the invention relates to methods for preventing, treating, or reducing systems of a disorder involving the autotaxin (ATX) pathway such as an inflammatory disease, autoimmune disorder, fibrosis or malignancy of the lung. Also disclosed are kits for performing the methods of the invention.

In one aspect, the invention features a method for preventing, treating, or reducing symptoms of an inflammatory disorder, autoimmune disorder, or fibrosis or malignancy of the lung in a subject such as a mammal. In one embodiment, the method includes administering to the mammal a sufficient amount of an autotaxin (ATX) inhibitor, to prevent, treat or reduce symptoms of the inflammatory disorder, autoimmune disorder, or the fibrosis or malignancy of the lung.

In another aspect, the invention features a method for the diagnosis of an inflammatory disorder, autoimmune disorder, or fibrosis or malignancy of the lung in a subject such as a mammal. In one embodiment, the method features obtaining a biological sample from the mammal and detecting an increase in the amount of autotaxin (ATX) or product thereof and/or a decrease in substrate levels compared to a control. In many embodiments, the increase is taken to be indicative of the presence of the inflammatory disorder, autoimmune disorder, or the fibrosis or malignancy of the lung in the subject.

In another aspect, the invention features a method for preventing, treating, or reducing symptoms of sepsis or an acute lung injury in a subject such as a mammal. In one embodiment, the method features administering to the mammal a sufficient amount of autotaxin (ATX) or a biologically active fragment thereof.

In another aspect, the invention features a method for preventing, treating, or reducing symptoms of an inflammatory disorder, autoimmune disorder in a subject such as a mammal. In one embodiment, the method features administering to the mammal a sufficient amount of a lysophohatidyl acid (LPA) signaling inhibitor, to prevent, treat or reduce symptoms of the inflammatory disorder or autoimmune disorder.

Further disclosed is a kit for performing one or more methods of the invention.

### BRIEF DESCRIPTION OF THE DRAWINGS

FIG. 1 shows increased ATX expression in SFs from animal models of RA. A) a series of eight images of cells showing ATX expression in wt (FIGS. 1A.1 through 1A.4) and hTNFtg SFs (FIGS. 1A.5 through 1A.8) shown after immunofluorescent staining. Cells were grown on coverslips, treated with a protein transport inhibitor (Golgi block) and were then stained for ATX. B) Increased ATX levels in mouse synovial fibroblast culture supernatants. Cell supernatants were subjected to SDS/PAGE and probed with an anti-ATX antibody. Data shown are representative of three separate experiments. C) TNF increases ATX expression from SFs. Real-time PCR reveals that ATX mRNA levels increase after TNF treatment of SFs. Cells were cultured in the absence or presence of TNF, total RNA was extracted and cDNA synthesis was performed using the MMLV reverse transcriptase. Values were normalized to B2M expression.

FIG. 2 shows ATX mRNA expression in various samples A) ATX mRNA is highly expressed in mouse arthritic synovial fibroblasts. ATX deregulated expression was confirmed using real time PCR. Wt and hTNFtg SFs were cultured, total RNA was extracted and cDNA synthesis was performed using the MMLV reverse transcriptase. Values were normalized to B2M expression. Data are means±SD of triplicate cultures. B) ATX is expressed from SFs but not from macrophages, thymocytes, spleenocytes or CD8 T-cells (upper image) relative to the ratio of expression of control B2m (lower image), as shown by RT-PCR analysis. C) Series of three images showing Flow cytometry analysis for wt and RA SFs (FIG. 2C.1), verifying the difference of expression. RA SFs were co-stained for vimentin and VCAM, and ATX⁺ cells were found to be also vimentin⁺ (FIG. 2C.2) and VCAM⁺ (FIG. 2C.3). D) TNF induces ATX expression from SFs. Semi-quantitative RT-PCR analysis shows that ATX expression is increased after TNF treatment in a dose-dependent manner (upper image) relative to the ratio of expression of control B2m (lower image).

FIG. 3 shows increased ATX expression in joints from animal models of RA A) a series of six images of showing that arthritic mouse joints overexpress ATX. Immunohistochemical analysis was performed in paraffin joint sections from 3 animal models of RA (FIGS. 3A.1 through 3A.3) and their wt controls (FIGS. 3A.4 through 3A.6). B) Increased ATX levels in the plasma of arthritic mice. Blood was collected from mice, plasma was isolated using EDTA and ATX concentration was determined by a direct ELISA. C) Decreased LPC levels in the plasma of arthritic mice. Blood was collected from mice, plasma was isolated using EDTA and LPC amounts were determined by LC/MS.

FIGS. 4A-4C show lipid levels in various plasma samples The levels of different lipids were measured in the plasma (FIG. 4A) of wt and hTNF^{+/-} mice using an HPLC column (FIG. 4B) and after performing mass spectrometry analyses (FIG. 4C).

FIG. 5 shows conditional ablation of ATX expression in synovial fibroblasts and chondrocytes results in decreased inflammation and synovial hyperplasia. A) is a series of two images showing disease development assessed in the hTNF-Tg197 animal model of RA. Mice were sacrificed, joints were removed and paraffin sections were stained with hematoxylin and eosin. Lack of ATX expression in the joints of Enpp2^{n/n}ColVICre^{30/-}hTNF^{+/-} mice (FIG. 5A.2) resulted in marked decreased inflammation (compare to FIG. 5A.1). B) Histopathological scores of arthritic joints that were given after microscopic examination. C) Correlation between disease severity index and ATX expression. D) is a series of two images showing reduced severity of CIA in ATX^{n/n}ColVICre^{+/-} mice. At 60 days post-primary immunization with CII, mice were sacrificed and their joints were removed and processed for histology. ATX^{n/n} (FIG. 5D.2) mouse joints examined were normal in appearance, with smooth intact articular cartilage and absence of inflammatory cell infiltrate. The joints of ATX^{+/+} mice (FIG. 5D.1) showed severe pathology, with cartilage erosion and synovial inflammation.

FIG. 6 shows an evaluation of recombination efficiency using PCR A) Evaluation of recombination efficiency using PCR analysis on DNA samples from whole joint tissue. B and C) Evaluation of recombination efficiency by performing ATX immunostaining of mouse synovial tissues. D and E) Reduced incidence and severity of CIA in Enpp2^{n/n}ColVICre^{+/-} mice. The incidence of arthritis is shown as cumulative percentage (FIG. 6D). Mean clinical scores (±SEM) of Enpp2^{n/n} and Enpp2^{30/+} mice (FIG. 6E), are shown with time following primary immunization with CII.

FIG. 7 Treatment of mice with a specific ATX inhibitor prevents the development of RA. PF-8380 was administered through oral gavage twice daily at 50 mg/Kg. Dexamethazone, as a positive control, was administered twice weekly. n=4-7

FIG. 8 shows treatment of mice with an ATX inhibitor prevents development of RA A) Schematic representation of the experimental design. B) Clinical severity of CIA in DBA/1 mice of the three different groups. C) Serum anti-CII IgG levels in CIA-immunized mice at day 49 of CIA. D) a series of six images showing sections of the severely arthritic joints (forelimb, FIGS. 6D.1, 6D.2, and 6D.3) of the non-treated group (FIGS. 6D.1 and 6D.4) and of the HLZ (FIGS. 6D.2 and 6D.5) and dexamethasone (FIGS. 6D.3 and 6D.6)-treated groups were stained for haematoxylin and eosin.

FIG. 9 shows a schematic representation of an experimental design for the treatment of mice with anti-ATX antibodies Schematic representation of the experimental design for the treatment of mice with anti-ATX antibodies in order to test their effect on RA development.

FIG. 10 shows that LPA stimulates SF activation and effector functions. LPA stimulates SF activation and effector functions. A) Effect of LPA on SF proliferation. Cells were cultured in the absence or presence of different LPA concentrations, [³H]thymidine was added and counts were measured in a scintillation counter. B) LPA effect on SF adhesion to fibronectin. Cells were incubated in the presence or absence of LPA and were stained with crystal violet after 4 hours. C) LPA effect on SF migration to ECM. Cells were added to Boyden chambers and were incubated in the presence or absence of LPA in order to migrate to fibronectin. D) is a series of three images shown SFs exhibit actin cytoskeleton rearrangement upon stimulation with LPA. Cells were grown on coverslips in the presence (FIGS. 10D.2 and 10D.3) or absence (FIG. 10D.1) of LPA for 4 hours, fixed and stained for F-actin with phalloidin-TRITC. E) Fluorescence after phalloidin staining was measured using a TECAN fluorescence plate reader. F) is a series of three bar graphs showing the effect of LPA on SF proinflammatory cytokine secretion for IL-6 and KC (FIG. 10F.1), MIP-1α and RANTES (FIG. 10F.2) and TNF-α (FIG. 10F.3). The supernatants from untreated WTSFs or treated with LPA for 4 hours were used as samples for the Lincoplex Luminex assay. G) Effect of LPA on MMP-9 expression from SFs, as shown with RT-PCR. H) LPA synergizes with TNF on SF activation of proliferation. Cells were treated in the presence of different concentrations of LPA and/or TNF and their rate of proliferation was measured after [³H]thymidine corporation. I) Stimulation of SFs by LPA is MAPK-mediated. The effect of specific MAPK inhibitors on LPA-induced and RA SF proliferation was examined after culture of wt and RA SFs were in the presence or absence of p38 MAPK, JNK, ERK MAPK or Rho kinase inhibitors (SB203580, SP600125, PD98059 and Y27632 respectively).

FIG. 11 shows detection of LPA receptor mRNA expression and various LPA effects A) Detection of LPA receptor mRNA expression from mouse SFs and BMDMs. B) Stimulation of SFs by LPA is ATX-mediated. Wt and RA SFs were cultured in the presence of LPC and/or recombinant ATX and proliferation rate was determined after [³H]thymidine corporation. C) Effect of LPA on BMDM cytokine secretion. BMDMs were cultured in the presence or absence of LPS or LPA and the levels of specific cytokines (IL-6 in FIG. 11C.1; KC in FIG. 11C.2; RANTES in FIG. 11C.3; and TNF-α in FIG. 11C.4) were measured in the culture supernatants using the Luminex technology.

FIG. 12 shows increased ATX expression in joints from RA patients A) is a series of two images showing ATX immunostaining performed on 4 μm-thick joint sections from RA (FIG. 12A.1) and OA (FIG. 12A.2) human patients as described in Materials and Methods. B) Quantification of ATX expression in RA and OA synovial tissue sections by using intensity score C) Increased ATX levels in sera of patients with RA. ATX concentration was determined by ELISA. D) Decreased LPC levels in the plasma of patients with RA. LPC amounts were determined by LC/MS.

FIGS. 13A-13H are a series of images showing increased ATX expression in an animal model of MS Increased ATX expression in demyelinated areas in an animal model of MS as assessed with immunocytochemistry staining with an a-ATX antibody (FIGS. 13A and 13E), GFAP antibody (FIG. 13B), CD11b antibody (FIG. 13F), Dapi stain (FIGS. 13C and 13G), and the merged images of FIG. 13A-13C (FIG. 13D) and FIG. 13E-13G (FIG. 13H).

FIG. 14 shows genetic ablation of ATX from oligodendrocytes attenuates development of EAE. Clinical score of EAE induction in the indicated mouse strains (A) and Clinical score of EAE induction in the indicated mouse strains (B).

FIGS. 15A-15L are a series of images showing that genetic ablation of ATX from oligodendrocytes attenuates the development of EAE H&E and Luxol staining of spinal cord sections of the indicated mouse strains.

FIG. 16A-16F are a series of images showing increased ATX expression in an animal model of pulmonary fibrosis

FIG. 17 shows increased ATX expression within the fibrotic lung (A) (FIGS. 17A.1 through 17A.4) a series of representative immunocytochemistry images with an a-ATX antibody on tissue microarrays containing 25 IPF (FIGS. 17A.3 and 17A.4) and 20 normal (FIGS. 17A.1 and 17A.2) lung samples. (B) Computerized image analysis of immunostained sections (C) ATX expression showed increased staining intensity in hyperplastic epithelial cells rather than fibroblasts, after computerized image analysis.

FIG. 18 shows conditional inactivation of Enpp2 in Clara cells of the bronchi results in attenuation of BLM-induced fibrosis (A) is a series of images showing representative H/E staining of lung sections from Enpp2^{n/n} (FIGS. 18A.1 and 18A.3) and CC10Enpp2^{n/n} mice (FIGS. 18A.2 and 18A.4) 14 days after BLM instillation (FIGS. 18A.3 and 18A.4). (B) Soluble collagen measurements. (C) Total cells counts. (D) BAL and (E) serum ATX levels as judged by an ELISA assay

FIG. 19 shows pharmacologic inhibition of ATX results in attenuation of BLM-induced fibrosis

(A) is a series of images showing representative H/E staining of lung sections in the indicated groups (FIGS. 19A.1: Sal+Veh; 19A.2: Sal+BrP; 19A.3: BLM+Veh; and 19A.4: BLM+BrP) 14 days after BLM instillation. (B) Soluble collagen determination (n=6). (C) Total cell counts (n=6). Bars represent mean values±S.D.

FIG. 20 shows pharmacologic inhibition of ATX results in attenuation of BLM-induced fibrosis (A) Representative HIE staining of lung sections in the indicated groups 14 days after BLM instillation. (B) Soluble collagen determination (n=6). (C) Total cell counts (n=6). Bars represent mean values+S.D.

FIGS. 21A-21AD shows altered phospholipid homeostasis in the lung upon BLM-induced pulmonary inflammation and fibrosis

FIG. 22 shows evaluation of recombination efficiency (A) PCR analysis of DNA samples isolated from lung tissue sections confirms the presence of the recombined Enpp2 allele only in the case of CC10Enpp2^{n/n} mice. Immunocytochemistry using either fluorescent- (B) or HRP-conjugated (FIG. 22B.1: ENpp2^{n/n}; FIG. 22B.2 CC10Enpp2^{n/n}) (C) with secondary antibodies against ATX (FIGS. 22C.1 and 22C.2 at 10× magnification and FIGS. 22C.3 and 22C.4 at 100× magnification) reveals the absence of ATX expression in the majority of bronchial epithelial cells in the lungs of naïve CC10Enpp2^{n/n} mice.

FIGS. 28A and 28B show ATX expression in type II alveolar epithelial cells Black arrows point to individual type II epithelial cells that express ATX protein albeit at lower levels than the Clara cells of the bronchi.

FIG. 24 shows reduced tumorigenesis in CC10Enpp2^{n/n} mice Surface tumors were counted by three blinded readers under a dissecting microscope and the average values are depicted (n=3-4, p=0.04).

FIGS. 25A-25J are a series of images showing representative H/E stained lung sections, depicting all neoplastic lesions caused by urethane (FIGS. 25C-25J). CC10Enpp2^{n/n} developed mainly small areas of AAH that rarely progressed to AD (FIGS. 25A, 25C, 25E, 25G, and 25I). Enpp2^{n/n} mice developed a full spectrum of neoplastic lesions (FIGS. 25B, 25D, 25F, 25H, and 25J).

FIG. 26 shows an evaluation of recombination efficiency (A) PCR analysis of DNA samples isolated from lung tissue sections confirms the presence of the recombined Enpp2 allele only in the case of CC10Enpp2^{n/n} mice. Immunocytochemistry using either fluorescent- (B) or HRP-conjugated (FIG. 26B.1: ENpp2^{n/n}; FIG. 26B.2 CC10Enpp2^{n/n}) (C) with secondary antibodies against ATX (FIGS. 26C.1 and 26C.2 at 10× magnification and FIGS. 26C.3 and 26C.4 at 100× magnification) reveals the absence of ATX expression in the majority of bronchial epithelial cells in the lungs of naïve CC10Enpp2^{n/n} mice.

FIGS. 27A and 27B show ATX expression by type II alveolar epithelial cells Black arrows point to individual type II epithelial cells that express ATX protein albeit at lower levels than the Clara cells of the bronchi.

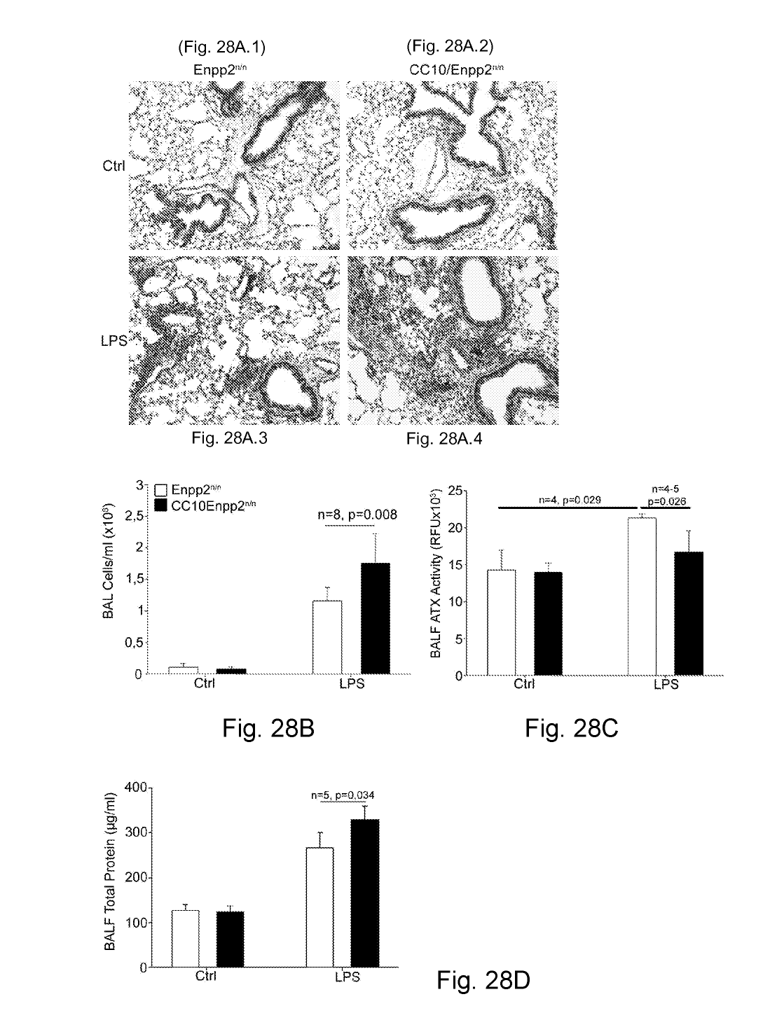

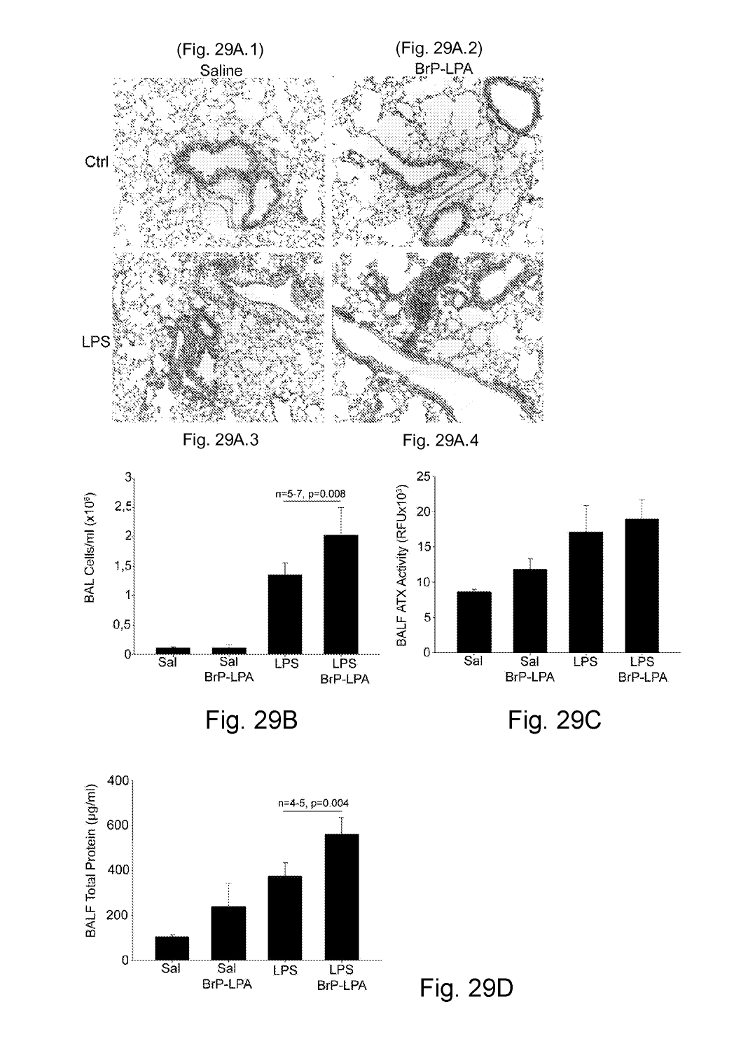

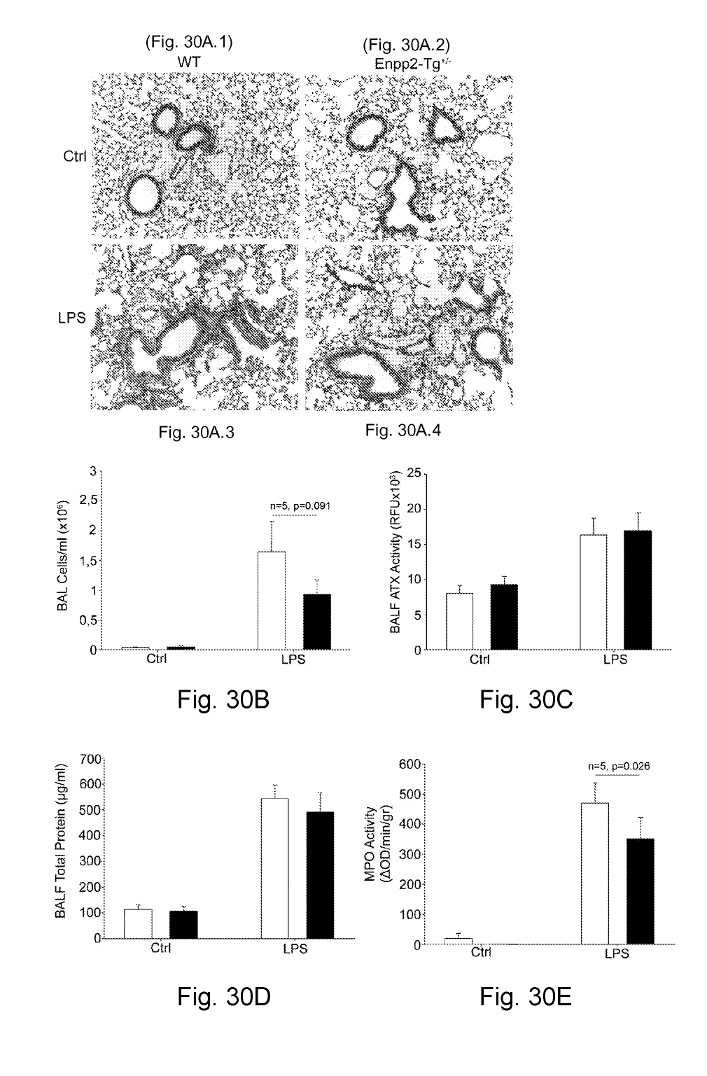

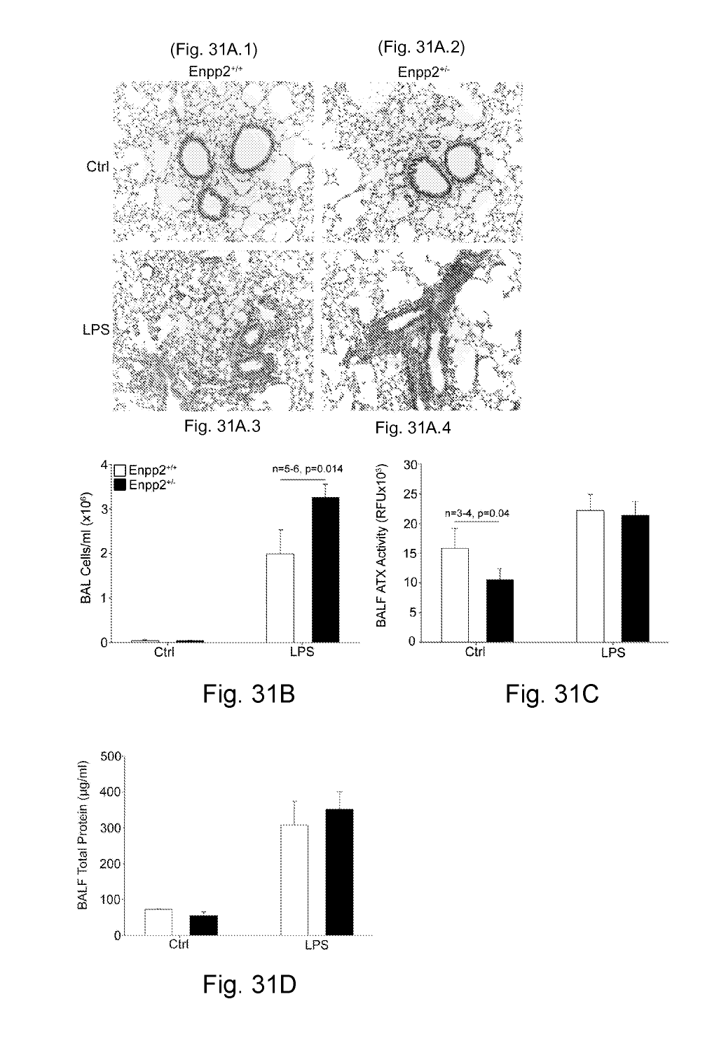

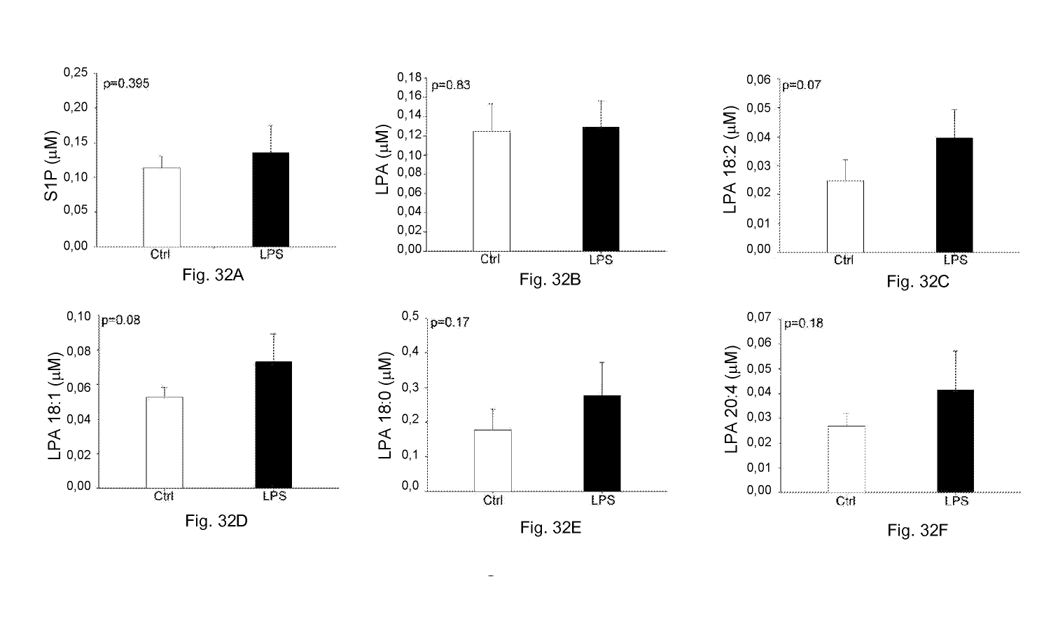

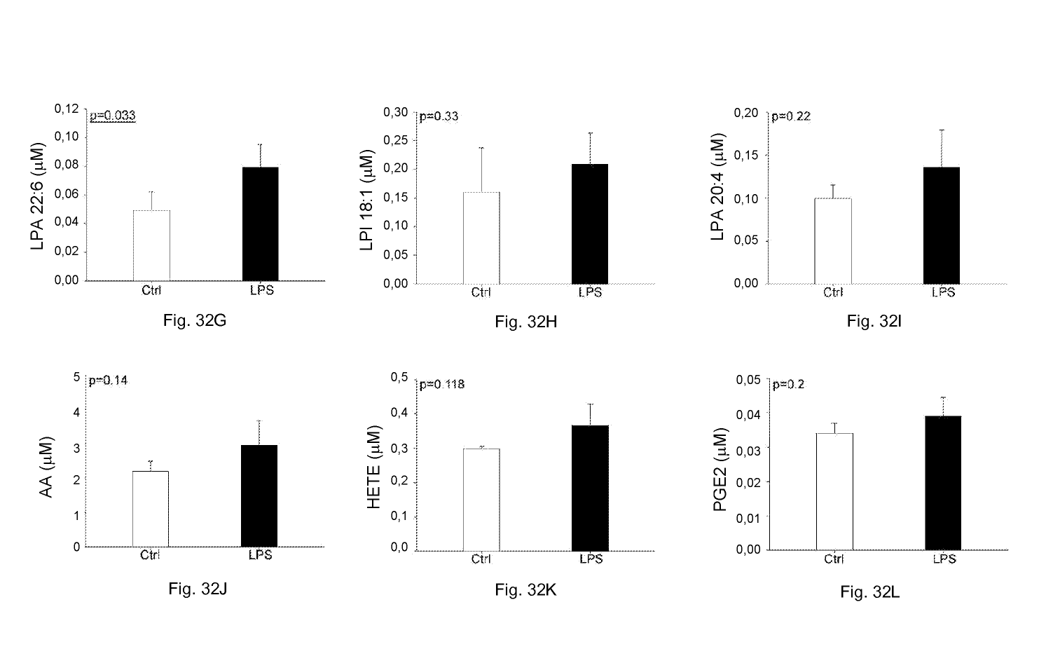

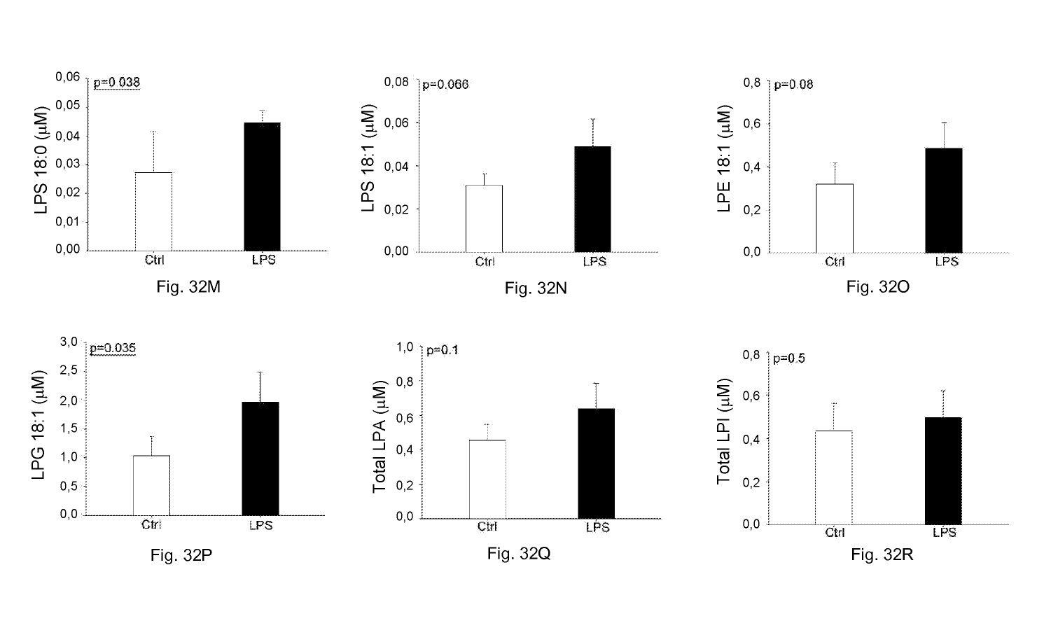

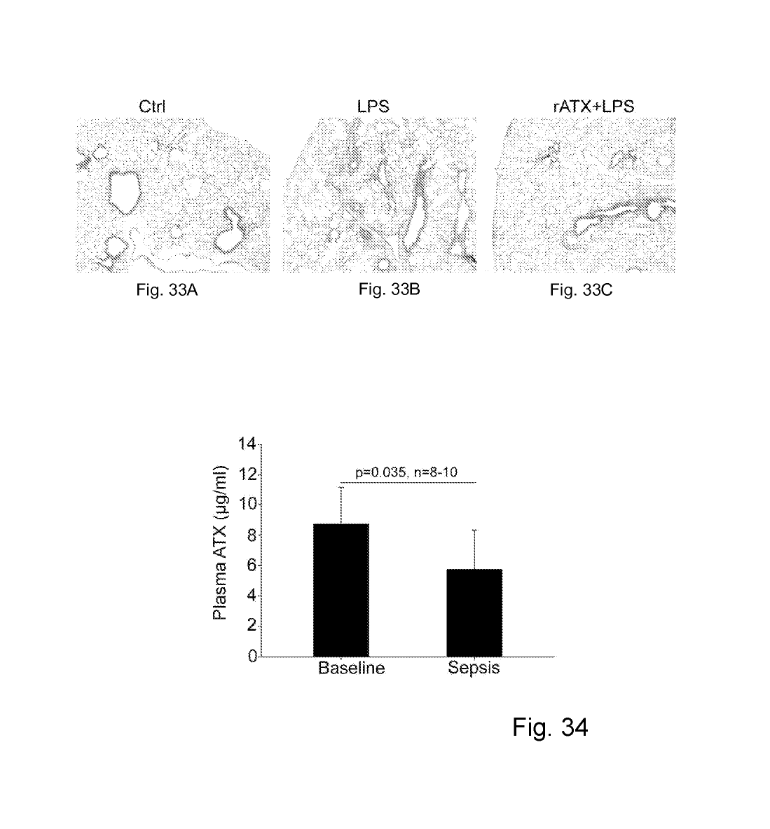

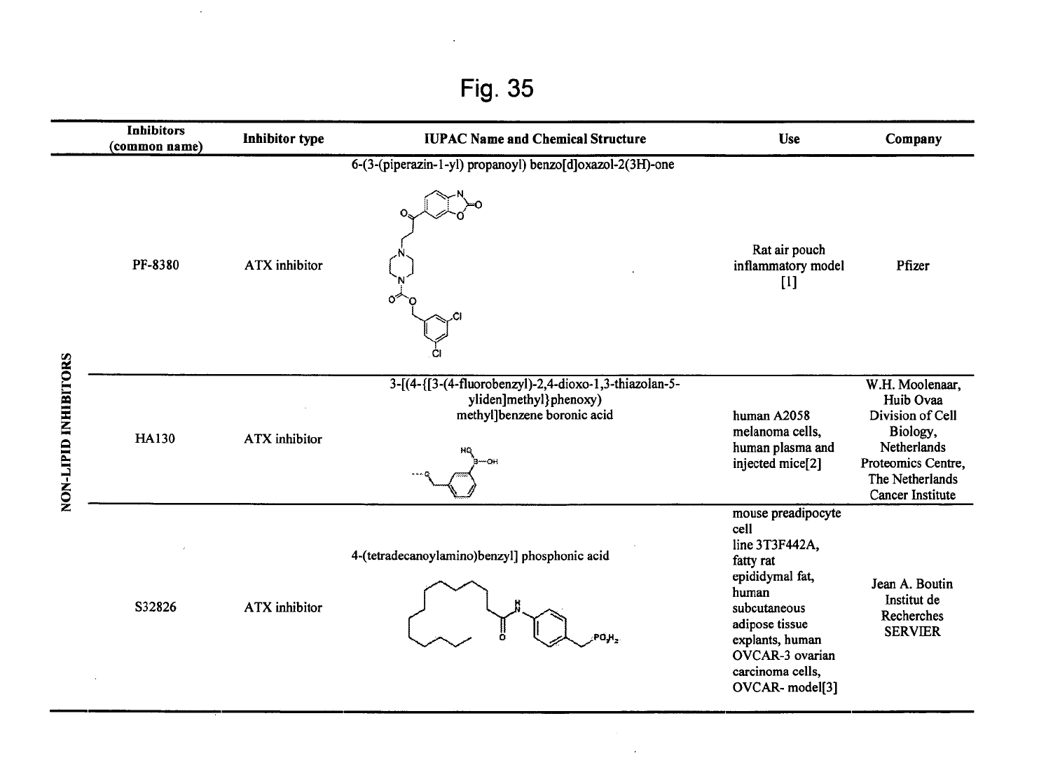

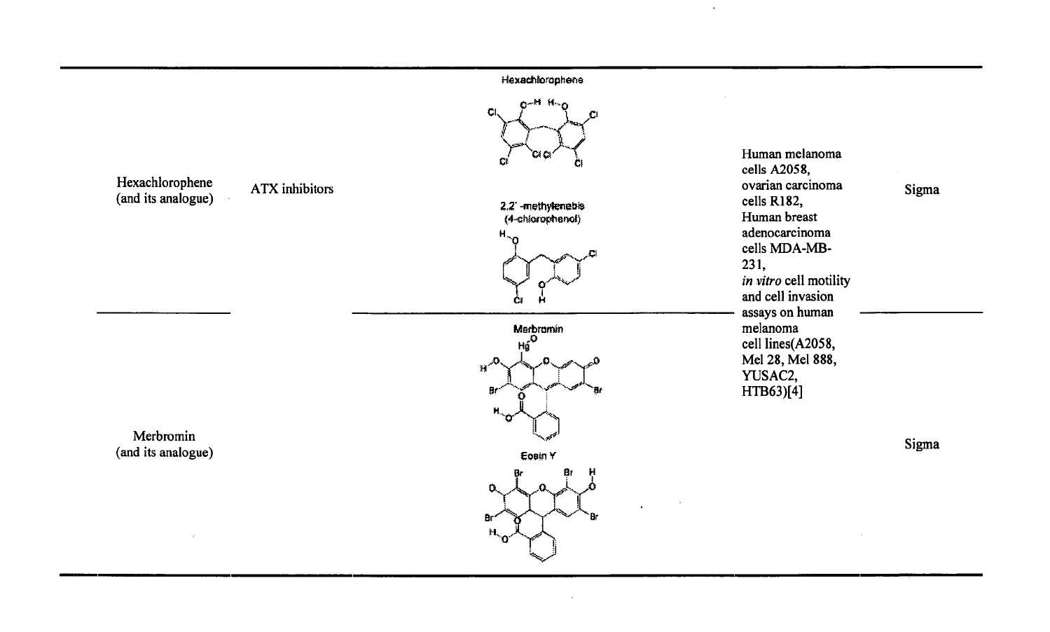

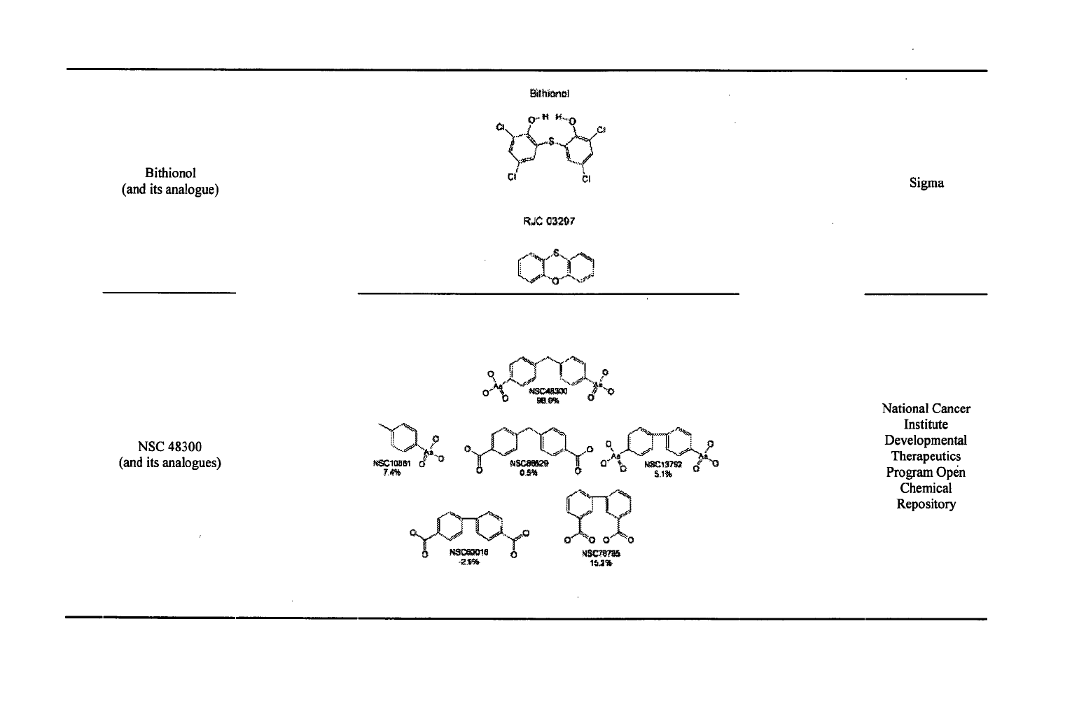

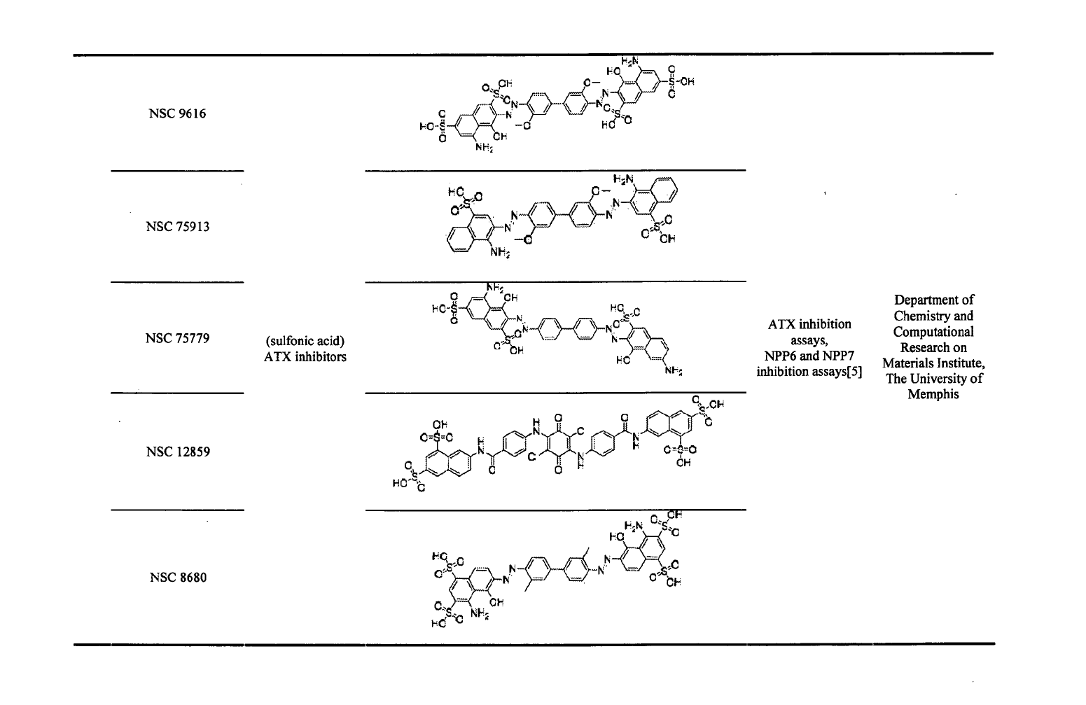

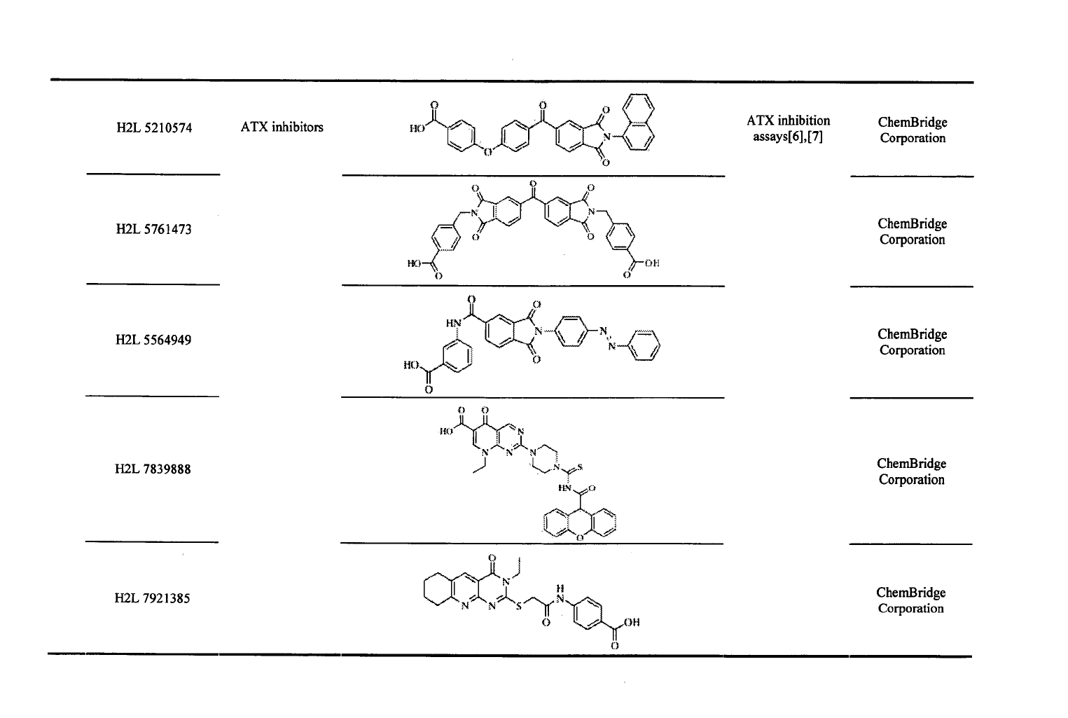

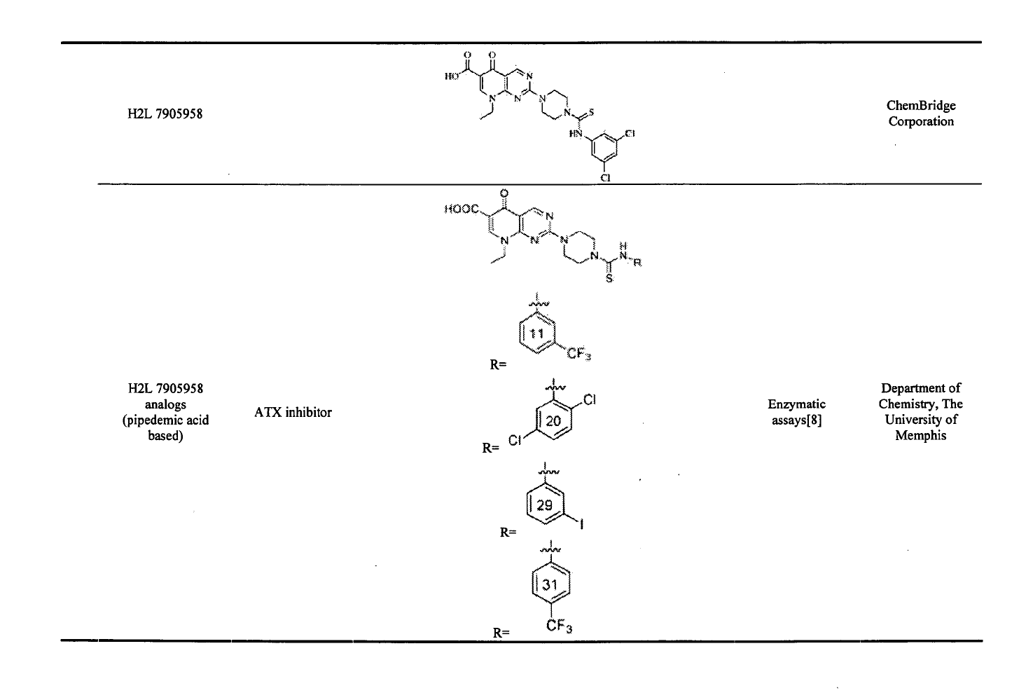

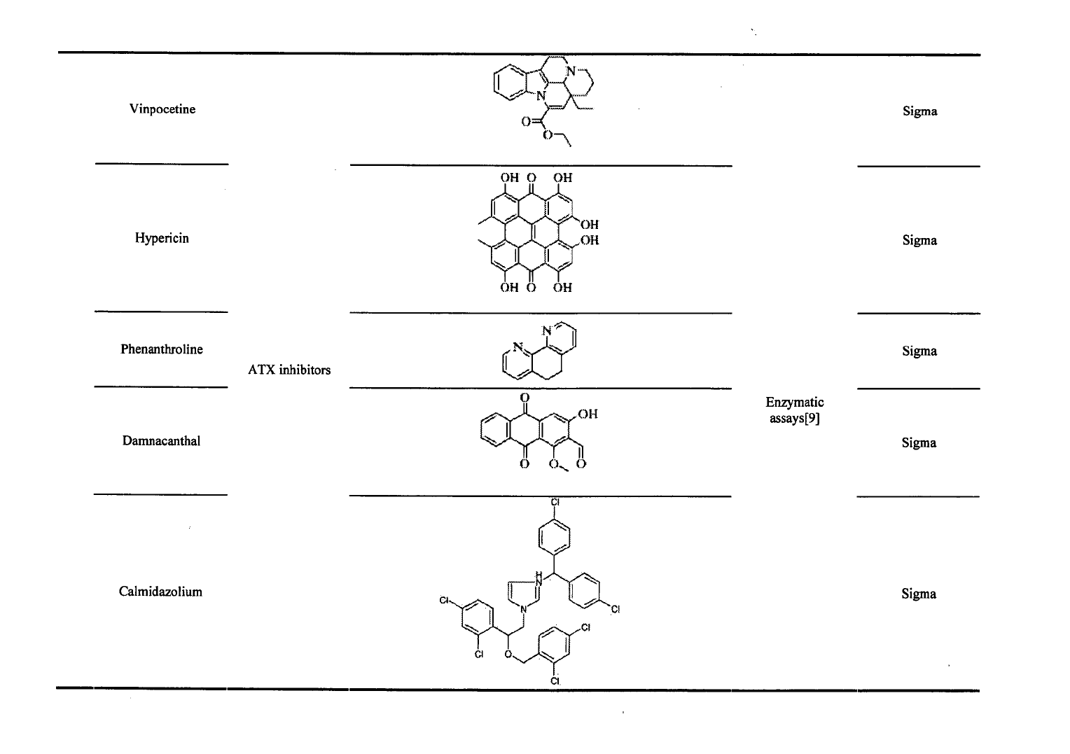

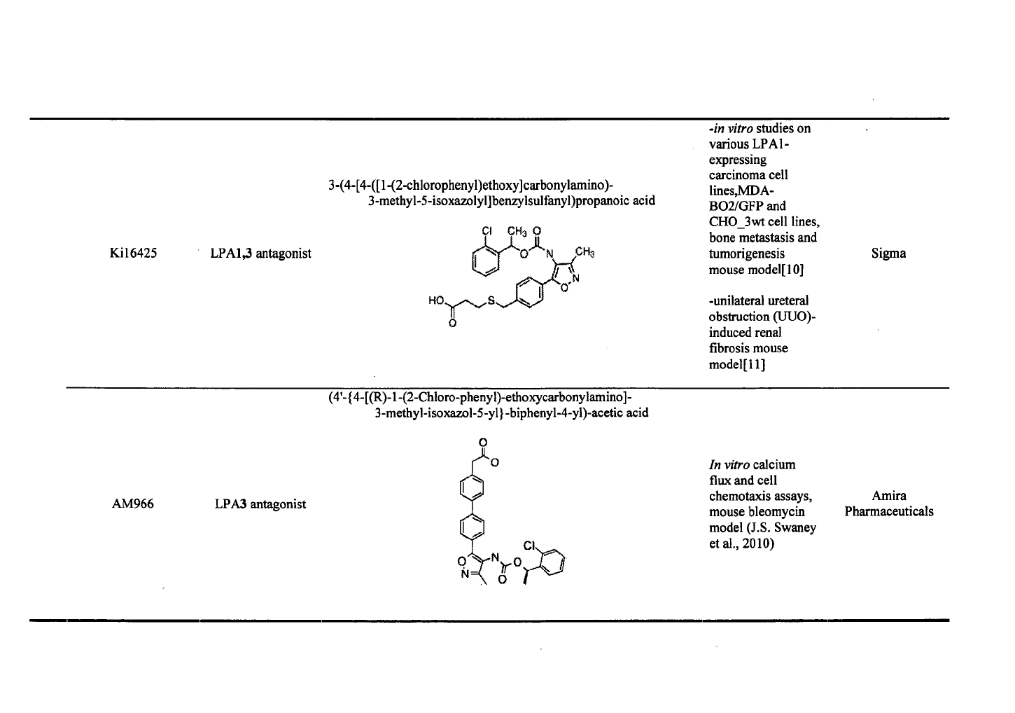

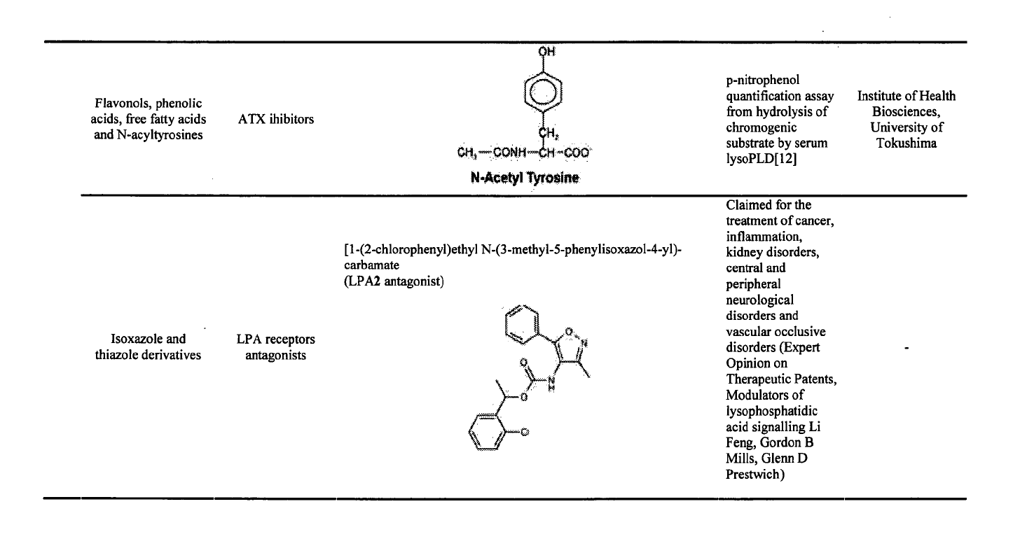

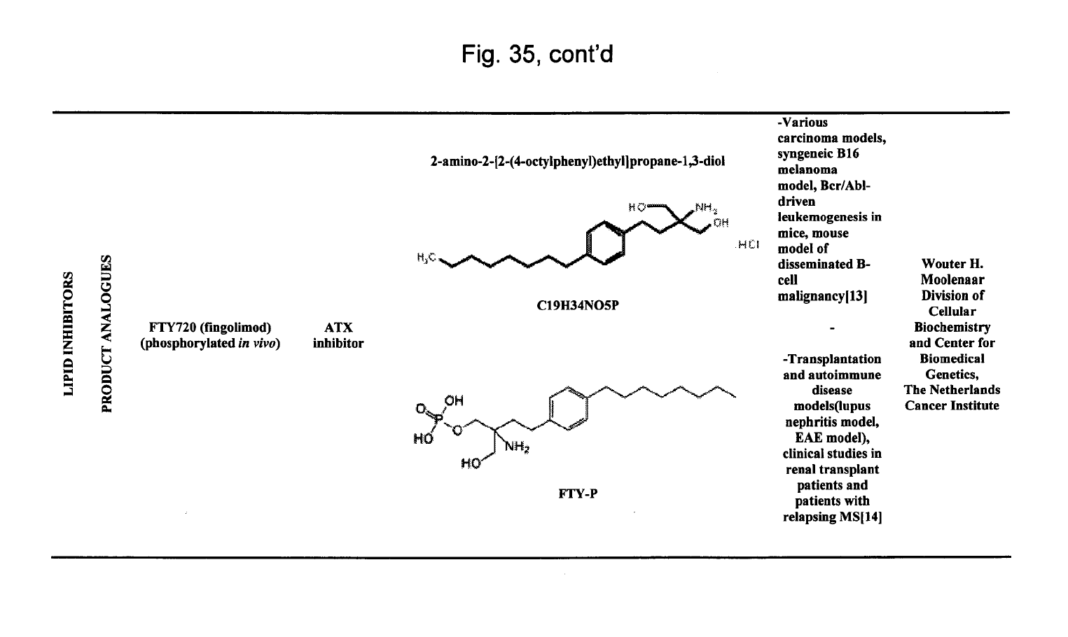

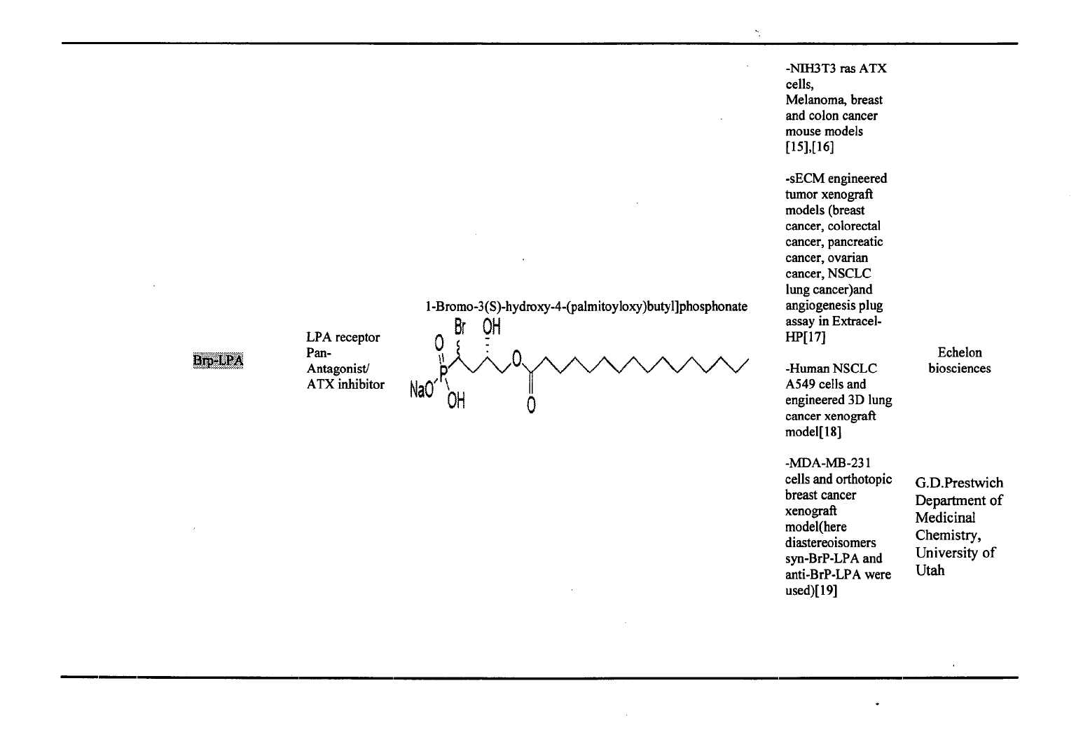

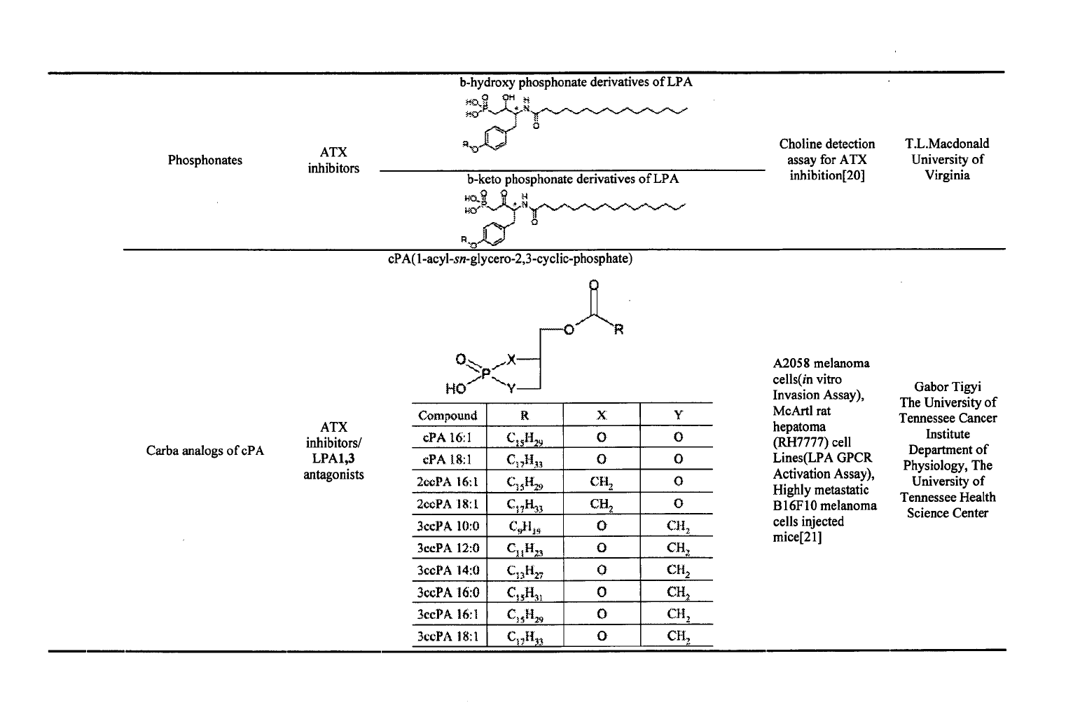

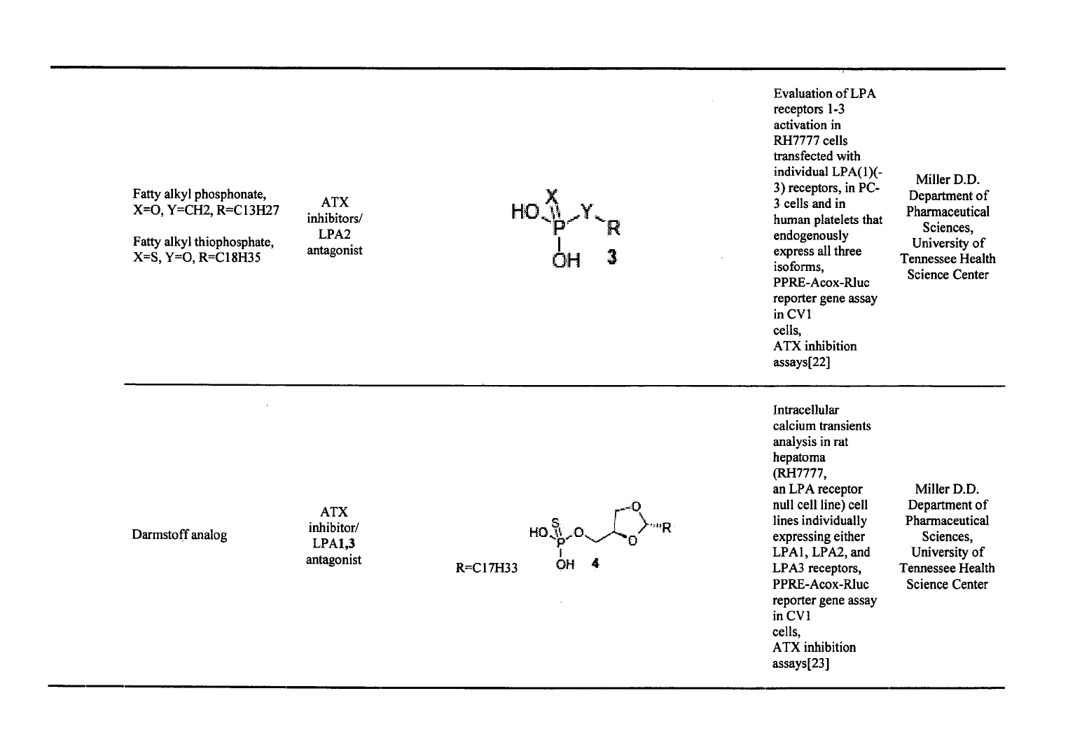

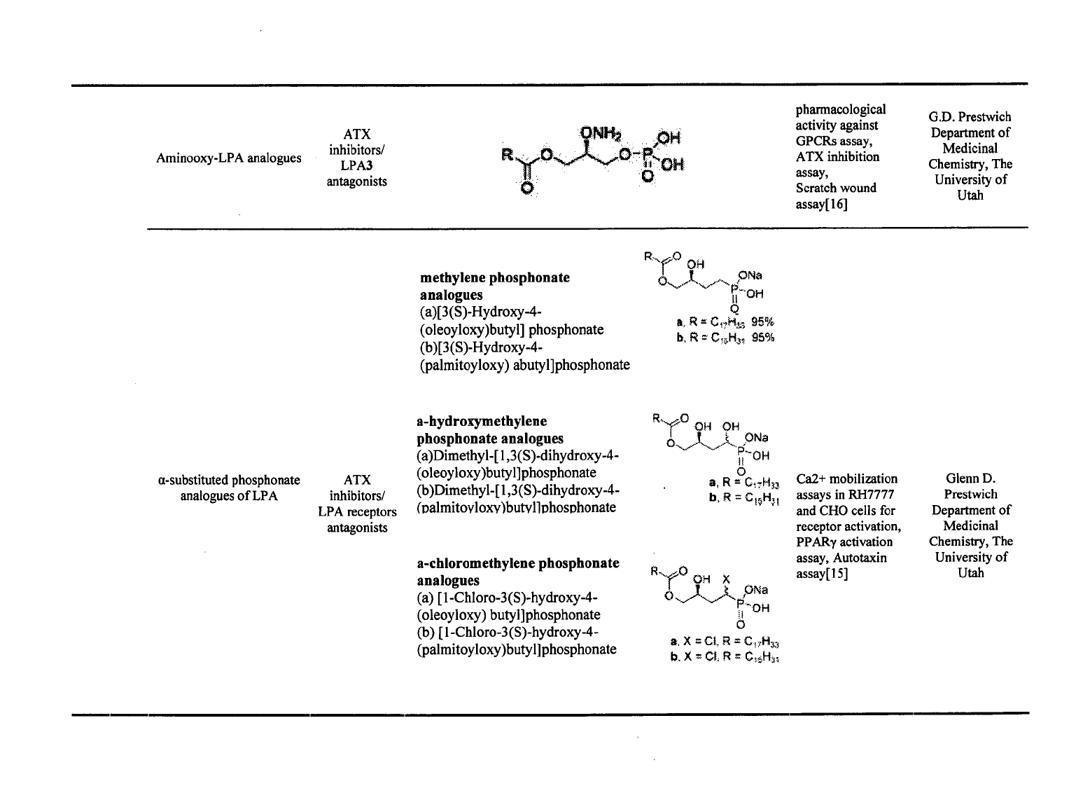

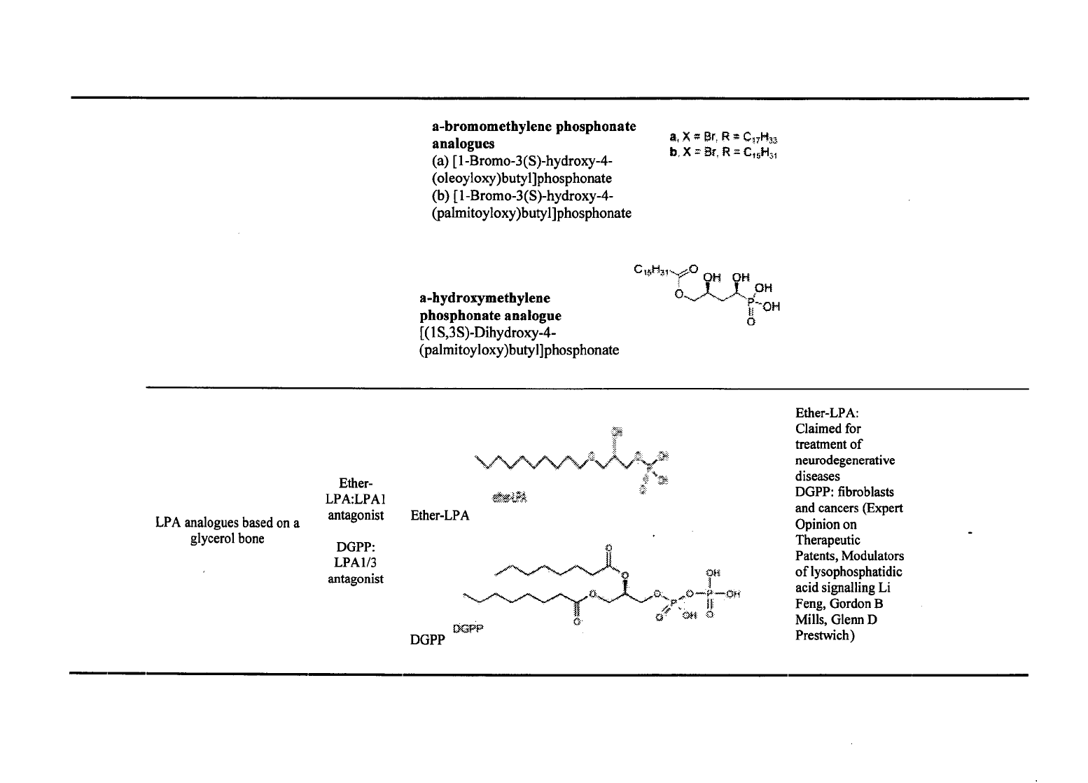

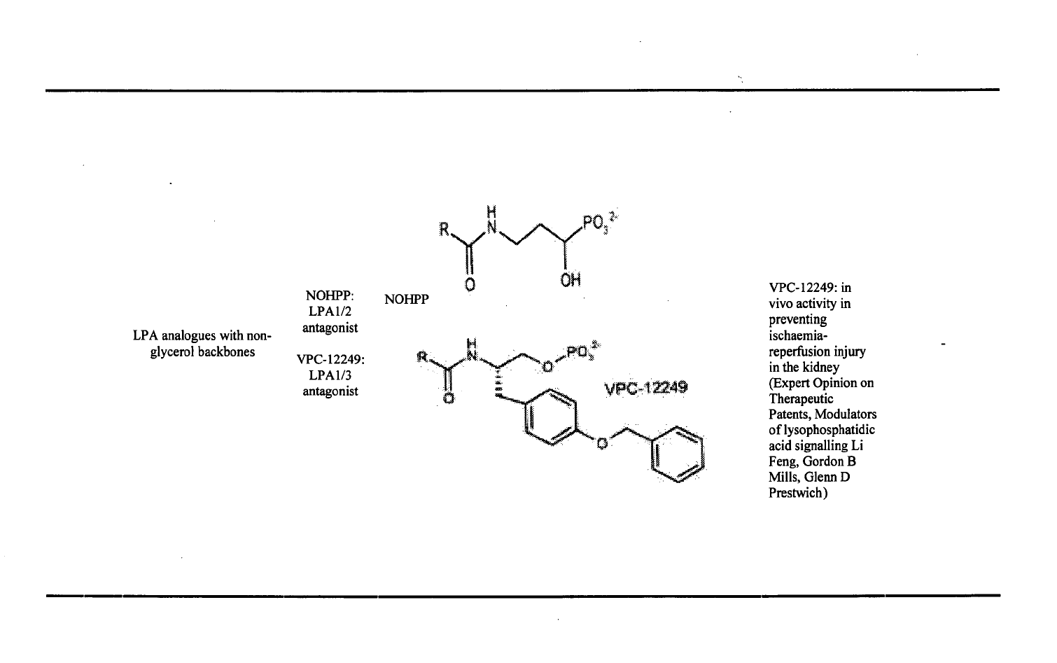

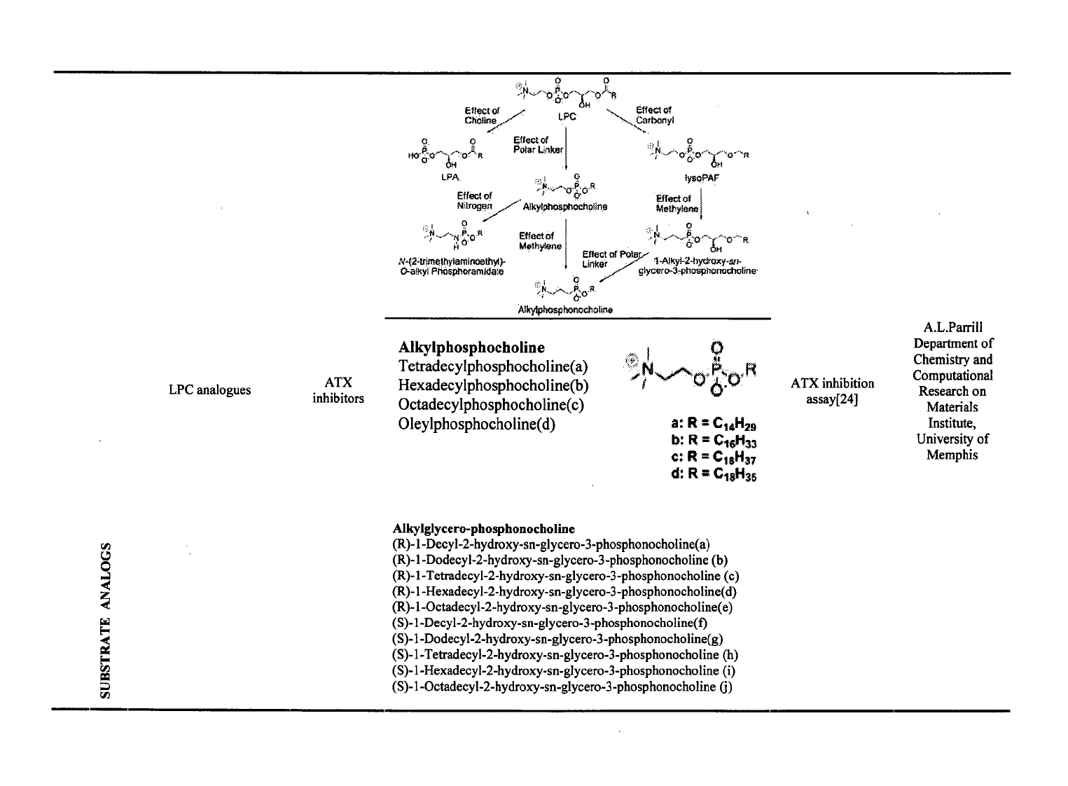

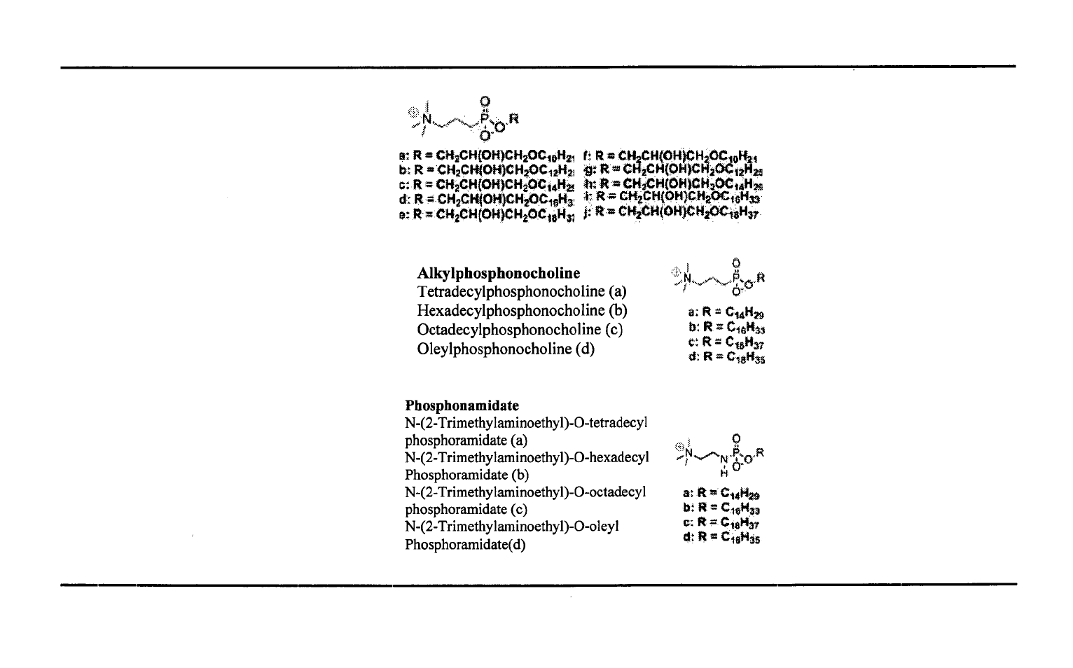

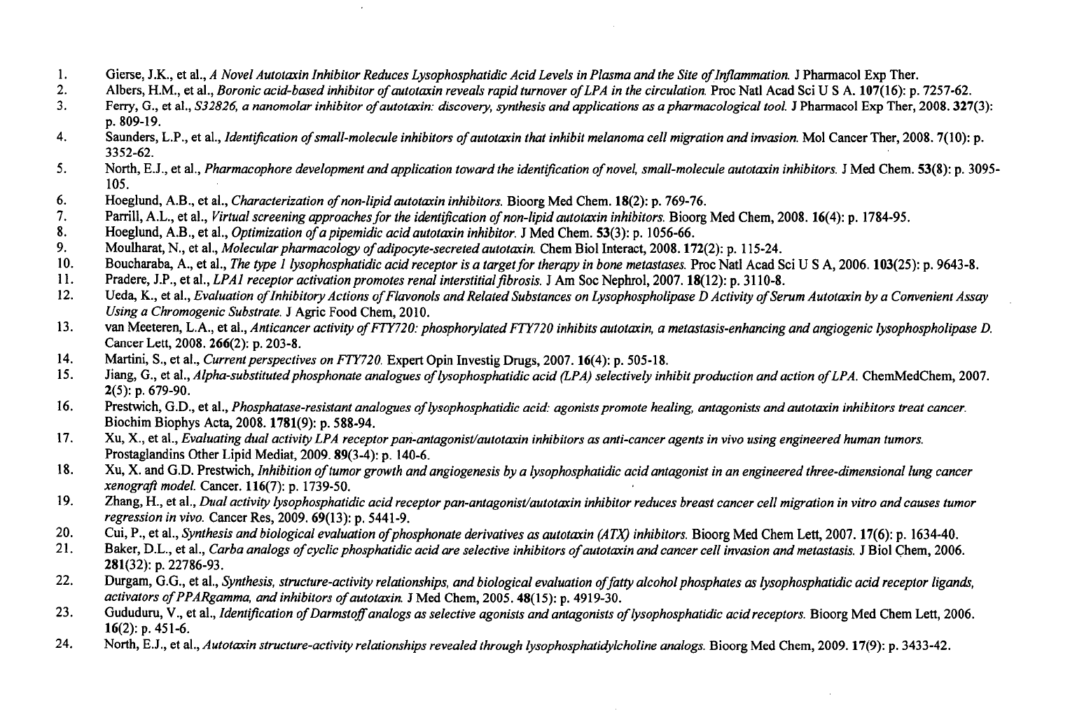

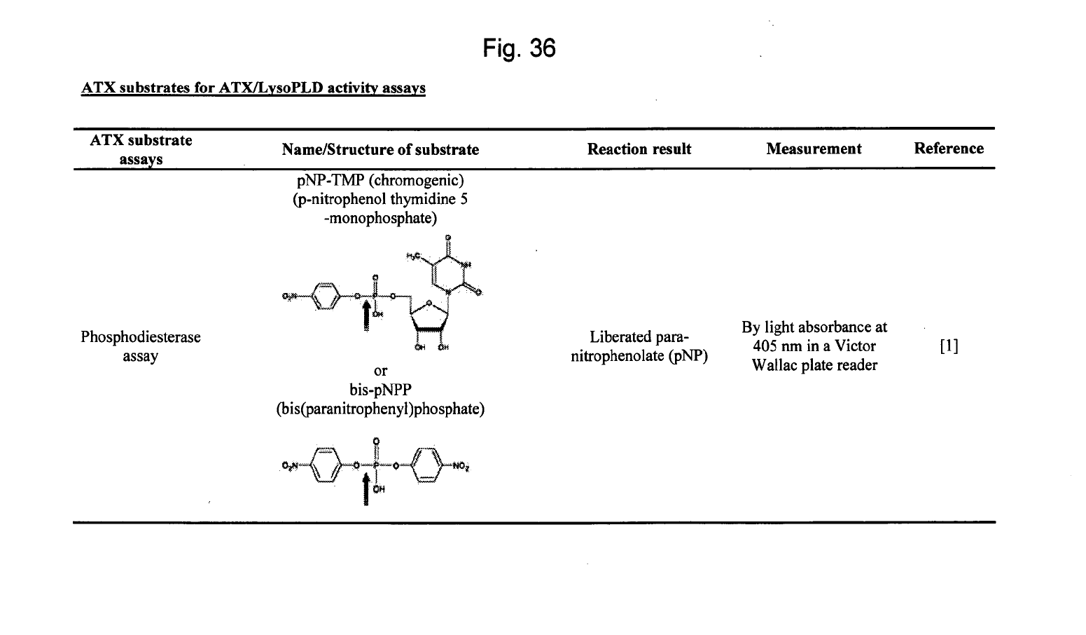

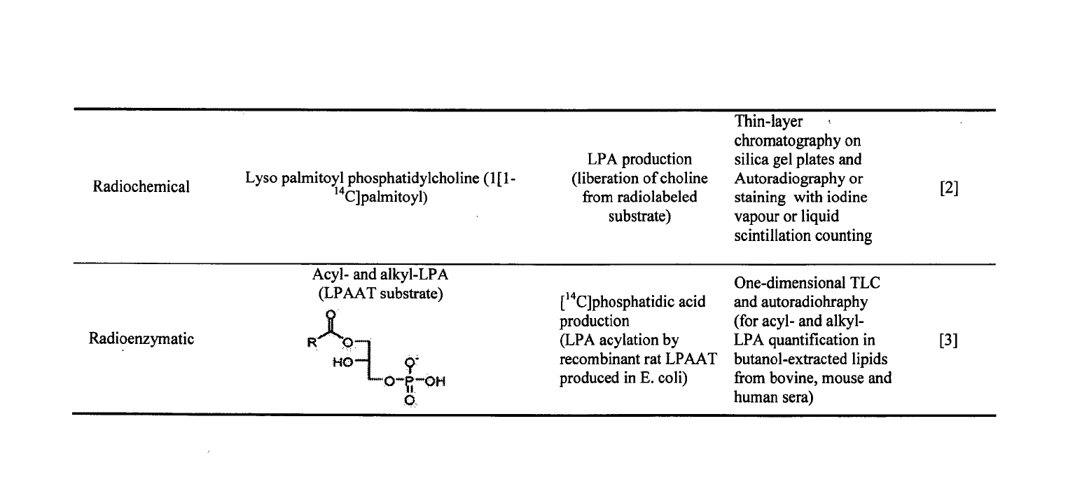

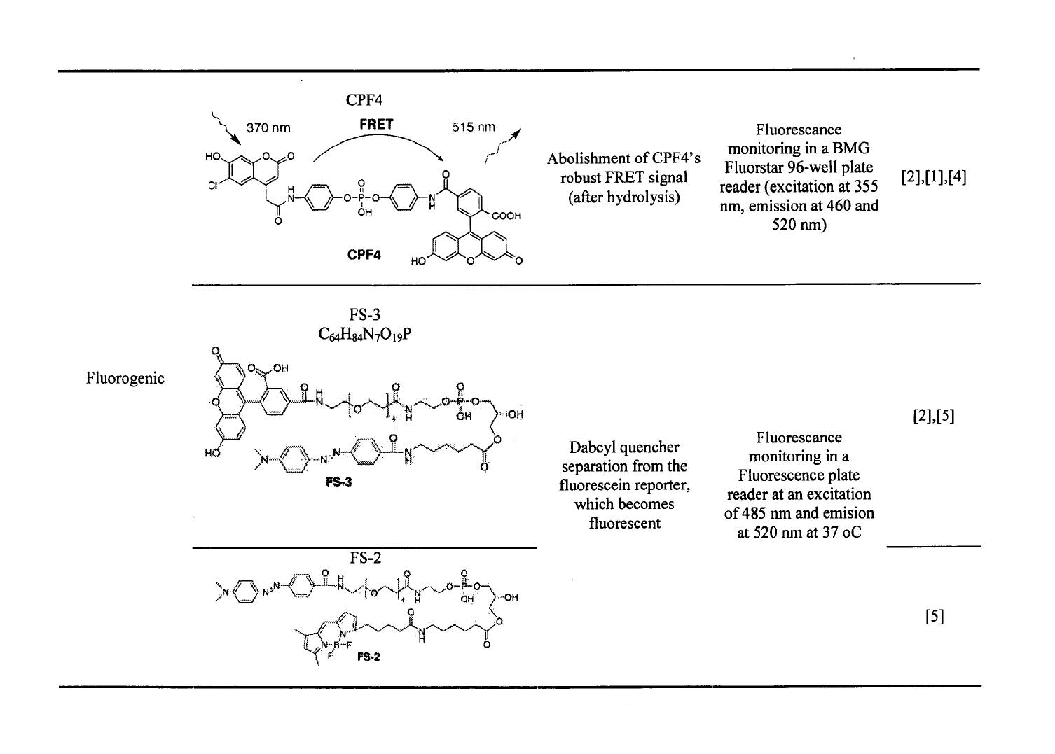

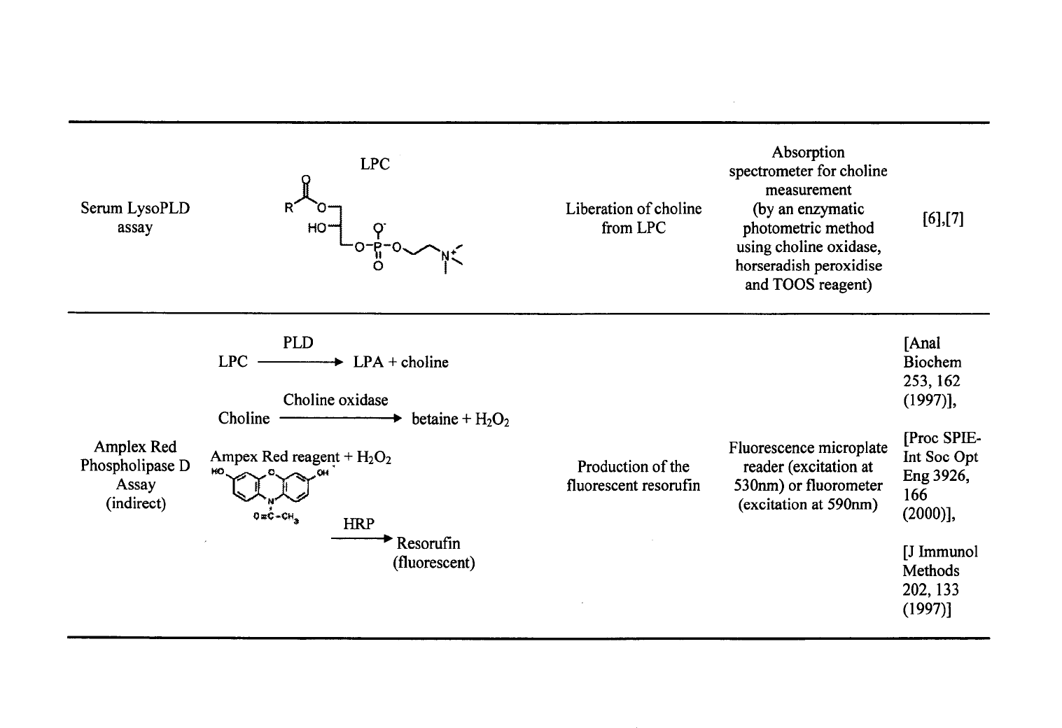

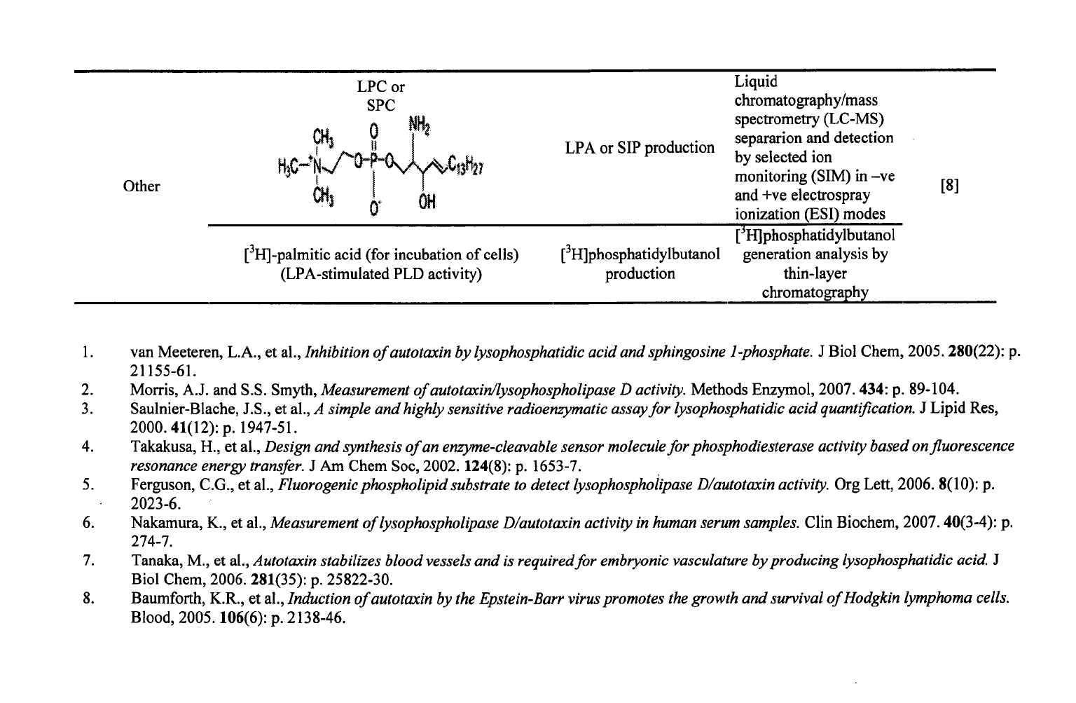

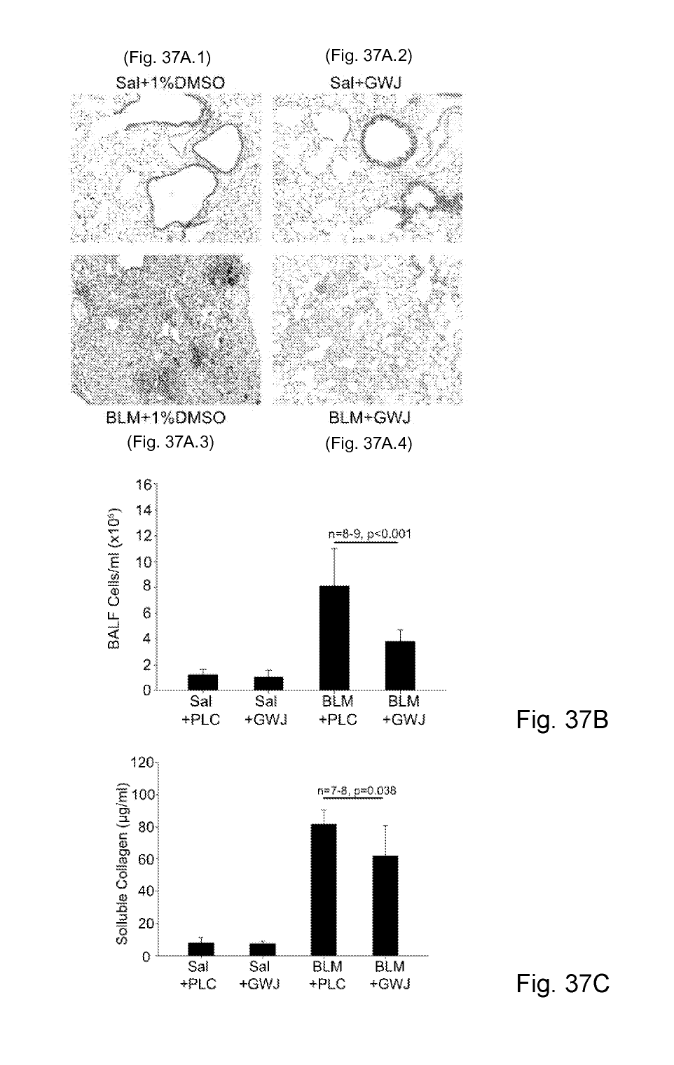

FIG. 28 shows that CC10Enpp2^{n/n} mice display increased neutrophil infiltration in the lungs upon LPS exposure (A) (FIGS. 28A.1-28A.4) are a series of images showing representative H/E stained lung sections from the indicated mouse strains. 24 h after LPS exposure (FIGS. 28A.3 and 28A.4, n=5). (B) Total cells counts in BALF. (C) BALF ATX activity as judged by the enzymatic cleavage of FS-3. (D) Total protein concentration of BALF. Bars represent mean values±S.D.

FIG. 29 shows pharmacologic inhibition of ATX exacerbates LPS-induced lung pathology

(A) (FIGS. 29A.1-29A.4) are a series of images showing representative H/E stained lung sections from the indicated mouse strains, 24 h after LPS exposure (FIGS. 29A.3 and 29A.4, n=5). (B) Total cells counts in BALF. (C) BALF ATX activity as judged by the enzymatic cleavage of FS-3. (D) Total protein concentration of BALF. Bars represent mean values±S.D

FIG. 30 shows systemic overexpression of ATX attenuates LPS-induced lung inflammation

(A) (FIGS. 30A.1-30A.4) are a series of images showing representative H/E stained lung sections from the indicated mouse strains. 24 h after LPS exposure (FIGS. 30A.3 and 30A.4, n=5). (B) Total cells counts in BALF. (C) BALF ATX activity as judged by the enzymatic cleavage of FS-3. (D) Total protein concentration of BALF. (E) MPO activity assay. Bars represent mean values±S.D

FIG. 31 shows that CC10Enpp2^{n/n} mice display enhanced pulmonary inflammation after LPS exposure (A) (FIGS. 31A.1-31A.4) are a series of images showing representative H/E stained lung sections from the indicated mouse strains. 24 h after LPS exposure (FIGS. 31A.3 and 31A.4, n=5). (B) Total cells counts in BALF. (C) BALF ATX activity as judged by the enzymatic cleavage of FS-3. (D) Total protein concentration of BALF. Bars represent mean values±S.D

FIGS. 32A-26R are a series of bar graphs showing shows a phospholipid analysis with LC/MS of BALF from LPS-challenged and control.

FIGS. 33A-33C are a series of images showing representative H/E staining of lung sections from mice that received LPS (FIG. 33A) or LPS plus rATX (FIG. 33B).

FIG. 34 shows ATX ELISA plasma sample from human patients at baseline and after sepsis development

FIG. 35 is a list of ATX inhibitors (lipid and non-lipid) and substrate analogs for use with the invention.

FIG. 36 is a list of ATX substrates for use with the invention FIGS. 37A.1-37A.4, 37B and 37C Pharmacologic inhibition of ATX results in attenuation of BLM-induced fibrosis. (A) (FIGS. 37A.1-37A.4) are a series of images showing representative H/E staining of lung sections in the indicated groups 14 days after BLM instillation. (FIG. 37B) Total cell counts (n=6). (FIG. 37C) Soluble collagen determination (n=6). Bars represent mean values±S.D.

### DETAILED DESCRIPTION

As discussed above, the invention relates to methods for preventing, treating, or reducing systems of a disorder involving the autotaxin (ATX) pathway in a subject such as a mammal. By the phrase “ATX pathway” is meant a biological component(s) that participates in or is part of the conversion of lysophosphatidylcholine (LPC) to lysophsphatidyl acid (LPA), for example, at least one of autotaxin (ATX, ENPP2), LPC, LPA, as well as receptors for LPC and/or LPA.

Examples of a preferred mammal for practice of the invention disclosed herein include rodents, rabbits, and primates such as a human patient.

As also discussed, the invention features a method for preventing, treating, or reducing symptoms of an inflammatory disorder, autoimmune disorder, or fibrosis or malignancy of the lung in a mammal that features administering to the mammal a sufficient amount of an autotaxin (ATX) inhibitor, to prevent, treat or reduce symptoms of the inflammatory disorder, autoimmune disorder, or the fibrosis or malignancy of the lung. In one embodiment, the inflammatory disorder is rheumatoid arthritis. In another embodiment, the autoimmune disorder is multiple sclerosis. A particular example of lung fibrosis is an interstitial lung disease, for instance, pulmonary fibrosis.

A variety of suitable ATX inhibitors can be used with the methods of the invention. Examples include a chemical compound or an antibody that specifically binds the ATX or an ATX binding fragment thereof such as a single-chain antibody (e.g., humanized). Other examples include an ATX substrate, ATX product analog, or a natural inhibitor. A particular example of an ATX product analog is Brp-LPA. Another preferred example of an ATX inhibitor is PF8380.

See FIG. 35 below for illustrative ATX inhibitors. See FIG. 36 for a list of exemplary ATX substrates at least some of which are detectable.

In one embodiment of the foregoing method, the method further includes administering a steroid or a non-steroidal anti-inflammatory drug (NSAID) to the mammal.

The invention further provides a method for the treatment of an individual suffering from or at risk of suffering from RA, said method comprising administering to said individual an inhibitor of ATX, thereby treating said individual for said disease. It was found that the level of ATX that is produced by synovial fibroblasts in RA individuals is regulated by tumor necrosis factor alpha (TNF). Addition of TNF to synovial fibroblast derived from healthy individuals led to increased expression of ATX. Moreover, inhibition of TNF in cultures of synovial fibroblasts harvested from an affected joint in an RA patient led to a reduced level of ATX being produced.

In one embodiment the method for treating the individual with RA or the individual at risk of suffering thereof further comprises administering to said individual an anti-TNF antibody for use in the treatment of RA. Examples of suitable anti-TNF antibodies are adalimumab, etanercept and infliximab (Taylor P C, Feldmann M. Anti-TNF biologic agents: still the therapy of choice for rheumatoid arthritis. Nat Rev Rheumatol. 2009 October; 5(10):578-82). Treatment with an anti-TNF antibodies often works well, however, sometimes in a subset of patients treated with the antibody develop other diseases such as tuberculosis (in some area's of the world), Lupus and other auto-immune diseases and possibly atherosclerosis. Using a combinatorial treatment with ATX and an anti-TNF antibody, as indicated herein above, it is possible to lower the anti-TNF antibody dose or reduce the frequency of administration, thereby reducing the incidence of the development of other diseases in the treated RA patients, when compared to a control group of RA patients receiving the recommended anti-TNF antibody dose without additional ATX treatment. The combinatorial treatment allows for similar beneficial effects with a concomitant but without the same amount of detrimental effects when compared to the mentioned control group. The lowered dose of anti-TNF antibody is preferable ½, more preferably ¼ or more preferably 1/10 of the dose of anti-TNF antibody recommended for use in the treatment of said RA patient when used as a stand-alone treatment. In another embodiment the invention provides a method for the treatment of an individual suffering from or at risk of suffering from RA as indicated herein, said method further comprising providing said individual with an anti-IL-6 antibody.

In the treatment of RA with anti-TNF antibodies it has become apparent that some RA-patients develop antibodies against the therapeutic antibodies. Also it has become apparent that some RA patients are refractory to anti-TNF antibody treatment. The patients that have an immune response to anti-TNF antibodies are preferably treated with a method of the invention. In these patients at least some of the downstream signaling effects of TNF can be prevented by a method of the invention. Also patients that are refractory to anti-TNF antibody treatment are preferably treated with a method of the invention. The reason why some patients are refractory to anti-TNF treatment is diverse and sometime not complete clear. For the refractory patients a method of the invention is particularly useful. A preferred group of refractory RA patients is the group that has developed inactivating antibodies against one or more of the therapeutic anti-TNF antibodies. Thus in a preferred embodiment said individual suffering from RA or at risk of suffering thereof comprises antibodies that can specifically bind an anti-TNF antibody that is used in the treatment of RA.

An individual is said to be refractory or non-responsive to anti-TNF antibody treatment when, upon treatment, the response in the individual is 10% or less that of a response of an individual that is responsive to anti-TNF antibody treatment. In rheumatoid arthritis (RA), inflammatory activity is typically not measured using one single variable. For this reason the Disease Activity Score (DAS) has been developed. The DAS is a clinical index of RA disease activity that combines information from swollen joints, tender joints, the acute phase response and general health.

The most advanced therapeutic approach for the treatment of RA is presently treatment with anti-TNF antibodies. These antibodies dampen the inflammatory symptoms in the affected joints of responsive RA patients. These antibodies have a long half-life in the circulation. Small molecules are typically cleared from the circulation more rapidly and it was therefore a question whether a significant effect on the inflammatory response could be attained with these small molecules. The present invention shows that such a dampening effect can be attained with the inhibitors of FIG. 35. The dampening effect is apparent upon pulsatile treatment of an individual suffering from RA or at risk of suffering from RA. Thus in a preferred embodiment wherein said ATX inhibitor is administered to said individual to achieve a pulsatile blockage of ATX in the circulation of said individual. Such an administration is herein referred to as pulsatile administration. Preferred routes of administration to achieve pulsatile administration are oral, subcutaneous and intravenous administration. A pulsatile administration typically entails at least one administration per day. Administrations that are more than 3 days apart are not considered pulsatile treatments as the levels of the drug in the circulation after three days is either not effective for at least a day (with rapidly clearing drugs, or still effective (with drugs that have a long half-life such as antibodies). In a preferred embodiment said pulsative blockage of ATX in the circulation is achieved by daily administration of said inhibitor to said individual.

The invention further provides an ATX inhibitor for use in a method for preventing, treating, or reducing symptoms of an inflammatory disorder or an autoimmune disorder in a mammal. The inflammatory disorder is preferably rheumatoid arthritis. The autoimmune disorder is preferably multiple sclerosis.

The ATX inhibitor is a chemical compound or an antibody that specifically binds the ATX or an ATX binding fragment thereof. In a preferred embodiment the ATX inhibitor is a synthetic ATX substrate, ATX product analog, or a natural inhibitor. Preferably said ATX inhibitor is a compound of FIG. 35. The ATX product analogue is preferably Brp-LPA in another preferred embodiment the ATX inhibitor is PF8380. An antibody that specifically binds ATX is preferably an antibody that inhibits the enzymatic LPC to LPA activity of ATX. The antibody fragment is a single-chain antibody that specifically binds ATX. The antibody or the fragment thereof is preferably human or humanized. Human antibodies can be generated in a various ways. For instance in transgenic mice that carry human immunoglobulin sequences. Also, libraries exist comprising variable domains of human antibodies. However, antibodies from mice or other organisms can be humanized by grafting the variable regions onto a human constant region. These antibodies can be further humanized by replacing variable region parts, for instance framework regions for the human parallel. Also human T or B cell epitopes can be removed from the antibody.

An inflammatory disorder of interest includes rheumatoid arthritis. An autoimmune disorder of interest is multiple sclerosis. In embodiments in which the disorder is fibrosis of the lung, the subject may have an interstitial lung disease such as pulmonary fibrosis.

In some invention embodiments, the method further comprises the step of contacting the sample with an antibody that specifically binds autotaxin (ATX) or an ATX binding fragment thereof and detecting the ATX as being indicative of the presence of the inflammatory disorder, autoimmune disorder, or the fibrosis or malignancy of the lung in the subject. In one embodiment, the detection step further comprises contacting the biological sample with a detectable ATX substrate under conditions sufficient to produce a detectable product, wherein presence of the detectable product is taken to be indicative of the amount of ATX in the biological sample.

In some invention embodiments, the ATX product is lysophosphatidic acid (LPA) or a metabolite thereof. In one embodiment, the method further comprises the step of contacting the sample with an antibody that specifically binds the LPA or an LPA-binding fragment thereof, and detecting the LPA or metabolite as being indicative of the presence of the inflammatory disorder, autoimmune disorder, or the fibrosis or malignancy of the lung in the subject.

In other invention embodiments, the ATX substrate is lysophosphatidylcholine (LPC) or a precursor thereof, and the method further comprises the step of detecting the LPC or precursor by performing one or more of chromatography, mass spectrometery; and detecting the LPC or precursor as being indicative of the presence of the inflammatory disorder, autoimmune disorder, or the fibrosis or malignancy of the lung in the subject.

In still other invention embodiments, the ATX product is lysophosphatidic acid (LPA) or a metabolite thereof; and the method further comprises the step of detecting the LPA or metabolite by performing one or more of chromatography, mass spectrometery and detecting the LPA or metabolite as being indicative of the presence of the inflammatory disorder, autoimmune disorder, or the fibrosis or malignancy of the lung in the subject.

In still other invention embodiments, the method will include the step of detecting a nucleic acid that encodes the ATX. In one embodiment, the method further comprises the step of performing a polymerase chain reaction (PCR) step to amplify the nucleic acid.

As discussed, the invention features a method for preventing, treating, or reducing symptoms of sepsis or an acute lung injury comprising administering to the mammal a sufficient amount of autotaxin (ATX) or a biologically active fragment thereof. In one embodiment, the acute lung injury is induced by a ventilator. In another embodiment, the acute lung injury is acute respiratory distress syndrome.

As also discussed, the invention features a method for preventing, treating, or reducing symptoms of an inflammatory disorder, autoimmune disorder. In one embodiment, the method includes administering to the mammal a sufficient amount of an lysophohatidyl acid (LPA) signaling inhibitor, to prevent, treat or reduce symptoms of the inflammatory disorder or autoimmune disorder. In one embodiment, the inflammatory disorder is rheumatoid arthritis. In another embodiment, the autoimmune disorder is multiple sclerosis.

A variety of suitable lysophohatidyl acid (LPA) signaling inhibitors can be used with the invention including an LPA inhibitor (small molecule or Ab), LPA receptor inhibitor (small molecule). See also FIG. 35.

### A. ATX is a Novel Biomarker and Therapeutic Target for Rheumatoid Arthritis

Increased ATX mRNA expression was detected in SFs from animal models of RA during differential expression profiling¹³, while human RA SFs were shown to express mRNA for both ATX and LPARs¹⁴⁻¹⁶. Here we show that ATX is overexpressed from activated SFs in arthritic joints, both in animal models and human patients. ATX expression was shown to be induced from TNF, the major pro-inflammatory factor driving RA. Furthermore and in order to assess the role of ATX and LPA signaling in RA pathophysiology, ATX expression was conditionally ablated specifically in SFs utilizing a conditional knockout mouse for ATX¹⁷ and a transgenic mouse strain expressing the Cre recombinase under the control of the ColVI promoter¹⁸. Disease development was assessed in well established animal models of RA. The lack of ATX expression in the joints resulted to marked decreased inflammation and synovial hyperplasia, suggesting an active involvement of the ATX-LPA axis in the pathogenesis of the disease. Similar results were also obtained with pharmacologic inhibition of ATX enzymatic activity and LPA signaling. A series of ex vivo experiments on primary SFs revealed that ATX, through LPA production, stimulates rearrangements of the actin cytoskeleton, proliferation and migration to the ECM, as well as the secretion of proinflammatory cytokines and MMPs. Moreover, the LPA effect was shown to be synergistic with TNF and dependent on the activation of MAPK cellular signaling pathways.

### B. ATX is a Diagostic Biomarker and Therapeutic Target in Multiple Sclerosis

As ATX was originally isolated from the supernatant of tumour cells, it was only natural to be associated with tumorigenesis¹. However, a number of studies have shown that ATX is also expressed in non-pathological conditions, throughout development, with high expression levels in the CNS among other tissues²⁸⁻³¹. In rodents, expression of ATX is first observed in the floor plate of the neural tube and the choroid plexus (where cerebrospinal fluid is produced) of the embryonic brain³². In the post-natal CNS, expression of ATX continues in the choroid plexus, which was recently been shown to be an essential organizing center for inducing dorsal neuron fates and sustaining neuron function³³ and was identified as a possible location of stem/progenitor cells³⁴. ATX mRNA was identified as highly upregulated during oligodendrocyte differentiation³⁵ and ATX protein expression is also apparent in maturing ODCs, temporally correlated with the process of myelination^{28,36}. Finally, in the adult brain ATX is expressed in secretory epithelial cells, such as the choroid plexus, ciliary, iris pigment, and retinal pigment epithelial cells^{28,31}, whereas there is evidence for ATX expression in leptomenigneal cells and cells of the CNS vasculature^{37,38}.

Although neurons and astrocytes do not seem to express ATX under physiological conditions, ATX is highly upregulated in astrocytes following brain lesion³⁹. Astrocytes represent a cell population that exhibit a dual nature with respect to regeneration. These cells are implicated in the chemo-traction of cells in the immune system and neuronal precursors, as well as having counter-adhesive effects in neurodegeneration and axon growth. In effect, two hall marks of reactive astrogliosis can be induced by LPA itself. hypertrophy of astrocytes and stress fiber formation⁴⁰. This may indicate an autoregulation loop of astrocytic activation, in which astrocytes upregulate the LPA-generating enzyme ATX and become activated by its metabolite LPA, while increased amounts of the metabolite inhibit the catalytic activity of ATX⁴¹.

ATX expression levels were shown to be elevated in glioblastoma multiform samples³⁷, and ATX was shown to augment invasiveness of cells transformed with ras, a key signaling molecule that promotes gliomagenesis⁴². ATX expression was also detected in primary tumor tissues from neuroblastoma patients⁴³ and retinoic acid induced expression of ATX in N-myc-amplified neuroblastoma cells⁴⁴. Furthermore, ATX mRNA expression was found to be elevated in the frontal cortex of Alzheimer-type dementia patients⁴⁵ and ATX protein levels were found deregulated in a animal model of MS (Experimental Autoimmune Encephalitis; EAE) at the onset of clinical symptoms²⁸. Interestingly, significant ATX expression was detected in the cerebrospinal fluid of patients suffering with Multiple Sclerosis (MS), completely lacking from the control samples⁴⁶, suggesting a role for ATX in maintenance of cerebrospinal fluid homeostasis during pathological/demyelinating conditions⁴⁷.

LPA signalling has been implicated in brain development, as high levels of LPA have been demonstrated in the brain, possible due to ATX activity, as well as from postmitotic cortical neurons than have been shown to synthesize and secret LPA⁴⁸. Lysophospholipids in general and LPA in particular can affect most neural cell types, impacting axonal and dendritic morphology^{48,49}, neuronal apoptosis⁵⁰, neural precursor cell survival⁵¹, process retraction in ODC precursors⁵², hypertrophy of astrocytes⁴⁰ and microglial activation and migration⁵³. Furthermore, LPA levels are increased during pathological conditions of the brain, such as in response to injury⁵⁴ cerebral ischemia, and following disruption of the blood-brain barrier⁵⁶. Moreover, LPA can induce hyperphosphorylation of the Tau protein⁵⁷ and high concentrations of intrathecally applied LPA cause receptor-dependent demyelination in dorsal root ganglia and PKCy up-regulation in the spinal cord⁵⁸.

LPA receptors are enriched in the CNS and their expression patterns suggest their potential involvement in developmental process including neurogenesis, neuronal migration, axon extension and myelination (Cervera et al., 2002; McGiffert et al., 2002). Noteworthy, only two receptors have the same spatiotemporal expression as ATX in the CNS⁵⁹⁻⁶¹. LPA₁ and S1P₅ are specific for ODCs, and their expression highly correlates with the process of myelination. LPA1 is expressed in restricted fashion within the neuroblasts of the neuroproliferatve Ventricular Zone (VZ) of the developing cortex, in the dorsal olfactory bulb, along the pial cells of neural crest origin, and in developing facial bone tissue. Expression is observed durin E11-E18, corresponding to a time period during which neurogenesis occurs. LPA1 expression is undetectable in the VZ after this point, to reappear during the first postnatal week within ODCs⁶². Notably, Schwann cells (the myelinating cells of the Peripheral Nervous System; PNS) express high levels of LPA1 early in development and persistently throughout life, consistent with LPA inducing their differentiation and myelin formation⁶³. Mice lacking LPA1 receptors show partial postnatal lethality with death of 50% of the LPA1 null mice within the first 3 weeks of age. The semi-lethality of LPA1 null mice is due to defective suckling which in turn is caused by defective olfaction. Surviving juvenile and adult LPA 1 null mice do display craniofacial deformities and increased apoptosis of Schwann cells in the sciatic nerve, confirming the previously observed survival effect of LPA on cultured primary Schwann cells⁵⁹. Mice lacking LPA2 receptors look phenotypically normal, while LPA1/LPA2 double knockout mice show hardly additional abnormalities compared to LPA1-deficient mice⁶⁰. Recently, LPA3 receptor knockout mice were shown to have significantly reduced litter size, which is caused by delayed implantation and altered embryo spacing. This also leads to delayed embryonic development, hypertrophic placentas shared by multiple embryos and embryonic death. Further insights into essential in vivo functions of LPA receptors must await the generation and analysis of LPA4 and LPA5 knockout mice and their combinations.

The above data strongly support a critical role for ATX and LPA signaling in neuronal development, oligodendrocyte differentiation and myelination, as well as possibly in the autoregulation of astrocyte activation. Moreover, the regulation of ATX and thus LPA production at local sites of CNS injury, inflammatory or autoimmune, could contribute to tissue homeostasis through the numerous effects of LPA. As demyelination and deregulated cerebrospinal fluid homeostasis are the hallmarks of Multiple Sclerosis, a role of ATX and LPA signalling in the pathophysiology of Multiple Sclerosis seems very likely.

### C. ATX is a Diagnostic Marker and Therapeutic Target for Pulmonary Fibrosis

Previous studies have demonstrated that mice lacking lysophosphatidic acid (LPA) receptor 1(LPAR1) were protected from Bleomycin (BLM)-induced pulmonary fibrosis and mortality, suggesting a major role for LPA in disease pathophysiology. 50% of circulating LPA is produced by the phospholipase D activity of Autotaxin (ATX) and the hydrolysis of lysophosphatidylcholine (LPC). Increased ATX expression has been previously reported in the hyperplastic epithelium of fibrotic lungs of human patients and animal models. Therefore, we hypothesized that genetic or pharmacologic inhibition of ATX activity would reduce local or circulating LPA levels and hence attenuate disease pathogenesis.

### D. ATX is a Therapeutic Target in Lung Cancer

Increased ATX expression has been detected in a large number of malignancies, including mammary^{71,72}, thyroid, hepatocellular⁷³ and renal cell carcinomas⁷⁴, glioblastoma⁷⁵ and neuroblastoma⁴³, as well as NSCLC⁷⁶ Strikingly, transgenic overexpression of ATX was very recently shown to induce spontaneous mammary carcinogenesis⁷². In accordance, in vitro ATX overexpression in various cell types promotes proliferation and metastasis while inhibiting apoptosis. Moreover, as an inducer of cell proliferation, migration and survival, LPA's actions are concordant with many of the “hallmarks of cancer”, indicating a role for LPA in the initiation or progression of malignant disease. Indeed LPA levels are significantly increased in malignant effusions, and its receptors are aberrantly expressed in several human cancers⁷⁷.

### E. ATX is A Diagnostic Marker and Therapeutic Agent in Acute Lung Injury

We hypothesized that genetic or pharmacologic inhibition of ATX activity would reduce circulating or local LPA levels and therefore modulate pathophysiology in animal models of sepsis. To test this hypothesis we have administrated aerosolized LPS (10 mg) via a custom made nebulizer in mice in which the Enpp2 gene was conditionally inactivated in Clara cells of the brochiolar epithelium (CC10Cre^{+/-}Enpp2^{n/n}, CBA-C57/Bl6) or in Enpp2^{n/n} mice that received an intratracheal instillation of ATX/LAPR inhibitor BrP-LPA (100 μg/mouse) prior to LPS exposure. ATX inactivation from Clara cells had as a result the enhancement of LPS-induced neutrophil infiltration to lungs (Enpp2^{n/n}: 1.25×10⁶±0.085 vs CC10Enpp2^{n/n}: 1.98×10⁶±0.5) as assessed by BAL fluid total cell counts and histopathology. Concomitantly BrP-LPA, a pan-LPAR antagonist and inhibitor of ATX's enzymatic action, when administered intratrachealy prior to LPS exposure, resulted in increased recruitment of neutrophils in the lungs when compared to Vehicle-treated control mice (Vehicle: 125×10⁶0.085 vs BrP-LPA: 1.9×10⁶±0.16). These results indicate that ATX and LPA signaling play an important role in modulating lung inflammation upon septic conditions and that ATX can be a therapeutic agent in acute lung injury.

The following materials and methods were used in Examples 1-6, below as needed.

Animals.

All mice were bred at the animal facilities of the Alexander Fleming Biomedical Sciences Research Center, under specific pathogen-free conditions. Mice were housed at 20-22° C., 55±5% humidity, and a 12-h light-dark cycle; water and food was given ad libitum. Mice were bred and maintained on C57BL/6J and on mixed C57BL/6×CBA genetic backgrounds in the animal facilities of the BSRC “Alexander Fleming”. Arthritic transgenic mice (Tg197, hTNF^{+/-} carrying the human TNF gene where the 3′ UTR was replaced by the corresponding one from b-globin) were maintained on a mixed CBA×C57BL/6 genetic background for over 20 generations. All mice were used in accordance with the guidance of the Institutional Animal Care and Use Committee of BSRC “Alexander Fleming”.

Reagents and Antibodies.

1-oleoyl-LPA (18:1) and 1-oleoyl-LPC (18:1) was purchased from Avanti Polar Lipids Inc. (Alabaster, Ala.). Extracellular signal-regulated kinase (ERK) inhibitor PD98059, p38 MAPK inhibitor SB202190, and JNK inhibitor SP600125 were obtained from Calbiochem (La Jolla, Calif.). Fatty acid-free BSA (BSA; fatty acid-free; Sigma-Aldrich), choline oxidase and peroxidase were from Sigma (Sigma-Aldrich, St. Louis, Mo.).

Cell Isolation and Culture.

Primary SFs were isolated from 6-8 week old mice as previously described. Briefly, mouse joint specimens were minced and digested with type IV collagenase. The released cells were grown in Dulbecco's modified Eagle's medium (DMEM) supplemented with fetal bovine serum (FBS) and penicillin-streptomycin. Isolated fibroblasts adhered on culture dishes were maintained under standard conditions (37° C. and 5% CO2) and used for the experiments between the third and fifth passages.

Cell Treatment.

Cells were starved with serum-free medium before treatment due to the presence of lysophospholipids in the serum Starvation medium is supplemented with 0.2% fatty-acid-free BSA. After routine starvation, cells were stimulated with various concentrations of the tested compounds (LPA, LPC, S1P or TNF) as indicated in details below. Where indicated cells were pre-treated with the inhibitors of ERK MAPK, p38 MAPK, JNK and Rho kinase for 3 hours prior to LPA stimulation. At the moment of cell treatment, the culture medium was replaced with fresh serum-free medium containing various concentrations of the tested compounds.

Proliferation Assay.

SFs were grown in 24-well tissue-culture plates in DMEM.

Preconfluent cell cultures were starved overnight, incubated for 24 h with LPA and the rest tested compounds and finally exposed to 0.5 μCi/ml of [³H]thymidine. Cells were then washed, harvested by trypsinization and blotted on a membrane. The radioactivity of incorporated [³H]-thymidine was determined by liquid scintillation counting.

Adhesion Assay.

This assay was performed on plates coated with human fibronectin. Briefly, cells were allowed to adhere to the substrate, and unbound cells were removed with sequential washes with PBS. Adhered cells were then stained with crystal violet, solubilized, and their absorbance was determined at 570 nm. The experiment was performed in triplicates.

Cell Migration Assay.

Modified Boyden chambers with 8-μm pores (Corning/Costar) were used where the lower surface of the membrane was coated with 10 μg/ml human fibronectin for 2 hours at 37° C. Cells were harvested with trypsin/EDTA, washed with PBS, and resuspended to 1×10⁶ cells per ml. The suspension was added to the upper chamber, and the cells were allowed to migrate at 37° C., 5% CO2, for 4 hours. Non-migratory cells were removed from the upper surface of the membrane while migratory cells in the lower compartment of the chambers were washed with PBS and stained with 0.2% crystal violet in 10% ethanol for 10 min. After extensive washing in H₂O, the cells were lysed and absorbance was measured at 570 nm with a SPECTRAmax photometer.

Oligonucleotide Array Hybridization.

Affymetrix Mu11K oligonucleotide DNA chipset (13179 murine transcripts) was hybridized (in duplicates) with fluorescently-labeled cRNA probes from total equimolar RNA of ex vivo SFs from 4 arthritic mice (Tg197 and TNFΔARE+/−) and their wild type (WT) controls.

RNA Extraction and RT-PCR Analysis.

Total RNA was extracted from subconfluent cultured SFs with the TRIzol reagent (Invitrogen Ltd, Paisley, PA4 9RF, UK Carlsbad, Calif. 92008) according to the manufacturer's instructions. RNA yield and purity were determined spectrophotometrically at 260/280 nm. cDNA synthesis was performed using the MMLV reverse transcriptase (Promega, Madison, Wis., United States Madison, Wis. 53711 USA). In all cases the normalization was performed in relation to the B2m internal control.

SQ PCR was performed by 20-27 cycles of denaturation at 95° C. for 30 s, annealing at 56-60° C. (depending on the Tm of each individual set of primers) for 30 s, and extension at 72° C. for 1 min, in a final volume of 20 μl. The products were separated by electrophoresis on 1.5% agarose gel and stained with ethidium bromide. Primer sequences (listed in the 5′ to 3′ direction, and designated as s, sense, and as, antisense) and product sizes (in bp) were as follows: Enpp2 (s, GTGAAATATTCTTAATGCCTCTCTG; as (SEQ ID NO: 1), GCCTTCCACATACTGTTTAAITTCC (SEQ ID NO: 2), 410), B2m (s, TTCTGGTGCTTGTCTCACTGA (SEQ ID NO: 3); as, CAGTATGITTCGGCTTCCCATTC (SEQ ID NO: 4), 104), MMP-9 (s, CAGATGATGGGAGAGAAGCA (SEQ ID NO: 5); as, CGGCAAGTCTITCAGAGTAGT (SEQ ID NO: 6), 222), LPA1(s, GAGGAATCGGGACA (SEQ ID NO: 7); as, TGAAGGTGGCGCTC (SEQ ID NO: 8), 227), LPA2(s, GACCACACTCAGCCTAGTCAAGAC (SEQ ID NO: 9); as, CAGCATCTCGGCAGGAAT (SEQ ID NO: 10), 200), LPA3(s, GCTCCCATGAAGCTAATGAAGACA (SEQ ID NO: 11); as, TACGAGTAGATGATGGGG (SEQ ID NO: 12), 188), LPA4(s, AGTGCCTCCCTGTTPGTCTTC (SEQ ID NO: 13); as, GCCAGTGGCGATTAAAGTTGTAA (SEQ ID NO: 14), 142), LPA5 (s, ACCCTGGAGGTGAAAGTC (SEQ ID NO: 15); as, GACCACCATATGCAAACG (SEQ ID NO: 16), 176), ATXKO-A1 CGCATITGACAGGAATTCTT (SEQ ID NO: 17), ATXKO-B2 TACACAACACAGCCGTCTCA (SEQ ID NO: 18). Quantitative real-time PCR was performed using the iCycler Iq Real-Time detection system and the IQ SYBR Green Supermix (Bio-Rad Laboratories, Hercules, Calif., United States), according to the manufacturer's instructions, for one cycle of 95° C. for 3 min, and 42 cycles of denaturation at 95° C. for 30 s and annealing at 56-58° C. for 45 s. PCR quality and specificity was verified by melting curve analysis and the expression level of the target mRNA was normalized to the relative ratio of the expression of B2m mRNA.

Primer sequences were the same as for the SQ PCR apart from Enpp2 sequences (listed in the 5′ to 3′ direction, and designated as s, sense, and as, antisense): (s, GACCCTAAAGCCATTATTGCTAA (SEQ ID NO: 19); as, GGGAAGGTGCTGTTTCATGT (SEQ ID NO: 20), 81 bp product size). Collagen-induced arthritia. C57BJ6 mice were injected intradermally at several sites into the base of the tail with an emulsion containing chicken collagen type II and heat-killed M. tuberculosis in IFA. The same injection was repeated as a boost 21 days later. All mice were between 8 and 12 weeks of age at the time of experimentation.

Immunohistochemistry for Mouse Joints.

Immunostaining was performed with peroxidase labeling techniques. Tissue sections were deparaffinized, and endogenous peroxidase activity was blocked by incubation in 1% peroxide. The sections were preincubated with 2% FBS in PBS-Tween for 30 minutes, followed by incubation overnight with the primary antibody. Sections were then washed in PBS-T and incubated for 30 minutes with horseradish peroxidase (HRP)-conjugated anti-rabbit IgG (1:1000 dilution in PBS-T). The sections were further washed with PBS-T. Finally, color was developed by immersing the sections in a solution of 0.05% 3,3′-diaminobenzidine (DAB; Sigma) and 0.01% hydrogen peroxide in PBS. The sections were counterstained with hematoxylin.

Immunohistochemistry for Human Joints.

Formalin fixed, paraffin-embedded sections of RA and OA synovial tissues slides were deparaffinised and treated at 80° C. for 30 minutes with citrate buffer (pH 6.0). After washing with H2O, the endogenous peroxidise was blocked with 1% H202 for 10 minutes. The slides were blocked with 2% Goat serum for 1 hour and incubated overnight at 4 C with a rabbit polyclonal anti-autotaxin 4 ug/ml (Cayman Chemical), IgG1 rabbit isotype control 4 ug/ml (Dako). After washing twice in PBS, the slides were incubated with their respective biotinylated secondary antibodies for 30 minutes. The signal was amplified with Horseradish peroxidise (HRP)-conjugated with streptavidin Vectastain Elite ABC kit (Vector, Burligame). Last, the slides were developed with a chromogenic substrate for peroxidise and counterstain with Haematoxylin. The expression of autotaxin was quantified semi-quantitatively on a 5-point scale, 0=no staining; 1=weak expression, single cells stained; 2=mild expression, limited areas stained; 3=moderate expression, weak overall expression and 4=strong expression, strong overall staining.

Immunofluorescence.

Cells were cultured on glass coverslips, grown in the presence of serum to subconfluence, and starved for 24 h by replacing the medium with serum-free medium containing 0.1% bovine serum albumin. Then the cells were treated with LPA and other components in serum-free medium for 4 h, fixed with 4% PFA and stained for F-actin with TRITC-conjugated phalloidin according to the manufacturer's protocol.

ATX ELISA.

To detect ATX in biological fluids, a direct ELISA assay was developed using the commercially available anti-phospholipase D polyclonal antibody (Cat. No. 10005375, Cayman chemical, USA). The bottom of a NUNC-IMMUNO 96 MicroWell Elisa plate was coated overnight with 100 μl of 1:200 diluted serum in 0.05 M carbonate/bicarbonate coating buffer, pH 9.5 (Cat. No. S7795/7277, Sigma, Saint Louis, Mich., USA), washed three times with 0.05% Tween-20 (Cat. No. P1379, Sigma, USA) in TBS and blocked with 0.1% Bovine Serum Albumin (Cat. No. A7888, Sigma, USA) in 0.05% TBST for 1 hour at RT. For each ELISA assay, 100 μl of two-fold serial dilutions of recombinant ATX protein ranging from 0.1 to 1.6 μg/ml were plated as standard curve and treated as all samples. The plate was then incubated with 0.5 μg/ml detection antibody for 1 hour and washed three times with 0.05% TBST. Autotaxin antigen was then detected with an anti-rabbit HRP-labeled secondary antibody (Cat. No. 4010-05, SouthernBiotech, USA) that was developed with TMB substrate (Cat. No. A7888, Sigma, USA). Readings were obtained at 450 nm. All samples and standards were assayed in triplicates.

SDS-PAGE/Western Blotting.

Cells and cell culture media were used to test for ATX protein expression. Cells were lysed in RIPA buffer and protein concentration was determined with a Bradford protein assay kit using BSA as standard. Protein samples (50 μg) were separated by 8% SDS-polyacrylamide gel electrophoresis and transferred to nitrocellulose membranes using the Bio-Rad protein transfer system. Primary antibody incubation was performed overnight in 5% (w/v) milk at 4° C. The membranes were then washed three times and incubated with appropriate horseradish peroxidase-conjugated secondary antibodies at room temperature. Membranes were washed for three times and antibody-antigen complexes were revealed using ECL.

Arthritic Score and Histopathology.

Paraffin-embedded joint tissue samples were sectioned and stained with haematoxylin and eosin. Arthritic histopathology was assessed (in a blinded fashion) for synovial hyperplasia, inflammation, cartilage destruction and bone erosion using a semi-quantitative (0-5) scoring as described previously.

Statistical Analysis.

The statistical significance of the differences between groups was determined by a paired Student's t test. The correlations were evaluated by linear regression analysis. P<0.05 was considered to be significant. All values are expressed as means±S.D (n=3). Group means were compared by Student's t test. Every experiment was repeated more than twice with similar results.

### Example 1

### Increased, TNF-Driven, ATX Expression from Arthritic SFs in Modeled RA

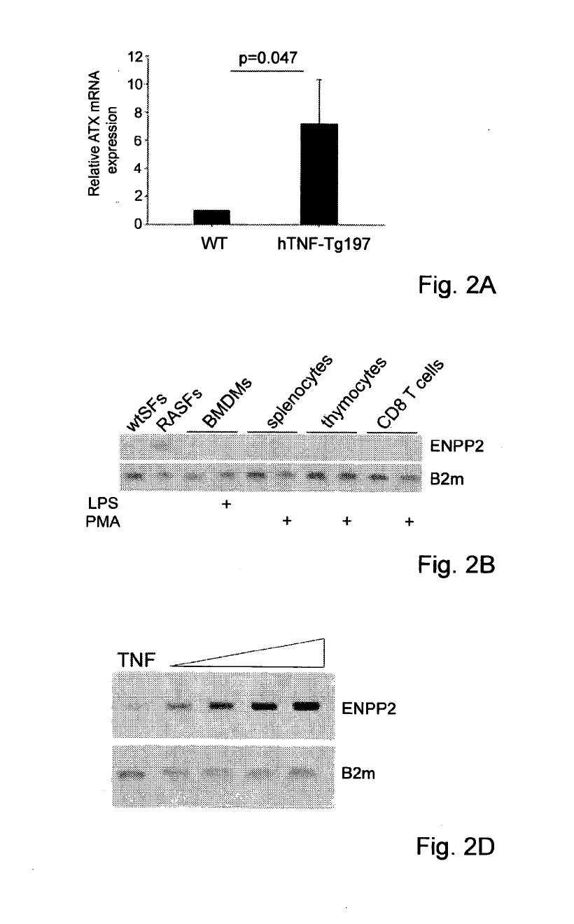

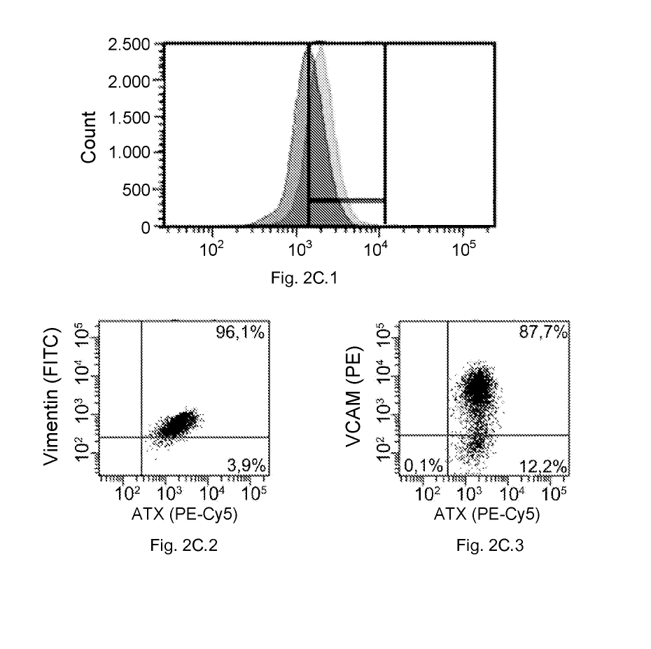

Increased ATX mRNA expression was detected in arthritic SFs from an animal model of RA (modeled RA, mRA; hTNF-Tg197^{19,20}) during differential expression profiling with DNA microarrays¹³. The finding was confirmed here with Real-Time RT-PCR in ex vivo-cultured, primary wt and mRA SFs isolated from the corresponding littermate mice (FIG. 2A). On the contrary no ATX mRNA expression was detected from primary macrophages (bone marrow derived; BMD) or T-cells (thymocytes, splenocytes or CD8⁺) even upon their activation (with LPS or PMA respectively; FIG. 2 B).

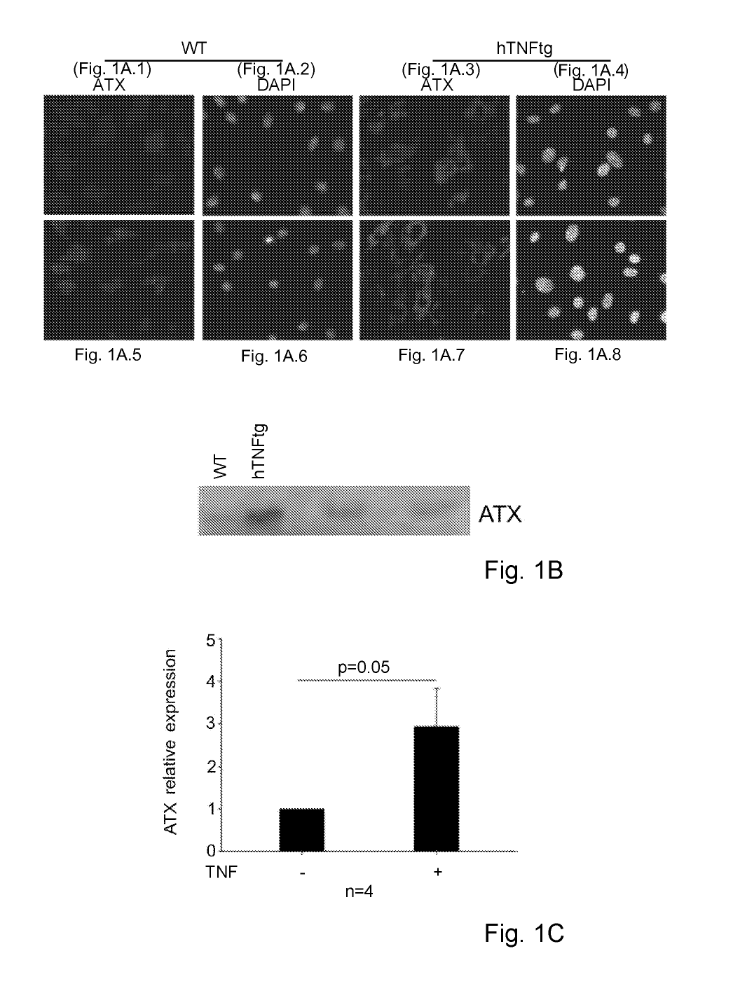

Immunostaining of wt and mRA SFs ex vivo further confirmed the increased ATX expression in mRA SFs (FIG. 1A). Moreover and as expected⁴, the expression pattern of ATX (speckled cytoplasmic) suggested that ATX is a secreted protein, consistent with the enhancement of ATX staining after treatment with Golgi block (FIG. 1A) and the expression of ATX in SFs supernatants (FIG. 1B). FACS analysis of cultured mRA SFs confirmed that the vast majority of cultured cells were fibroblasts (vimentin⁺) and that both intimal and sub-intimal (VCAM⁺ or VCAM⁻ respectively) SFs express ATX (FIG. 2C). More importantly, TNF—the major proinflammatory cytokine driving (m)RA development in vivo, was shown to induce ATX expression from SFs (FIG. 1C and FIG. 2D).

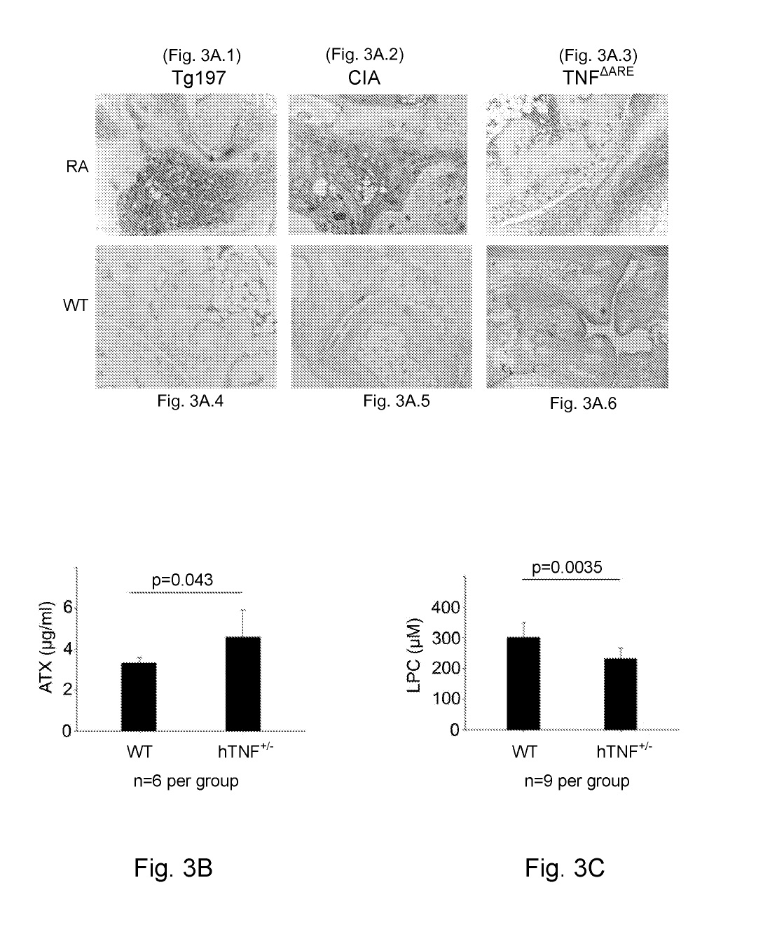

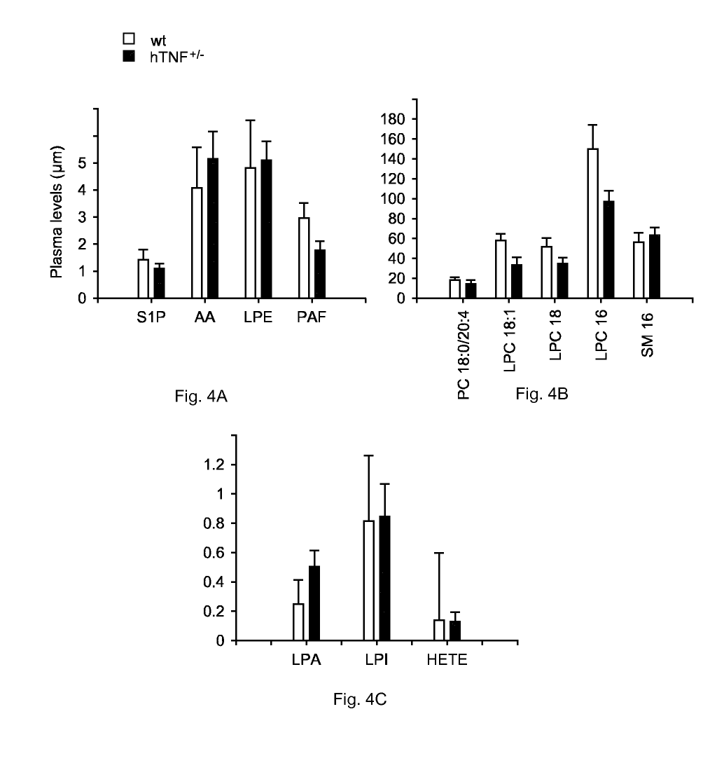

To confirm the results in vivo, ATX expression was next assessed with immunostaining of the arthritic joints from three animal models of RA: hTNF-Tg197^{19,20}, Tnf^{ΔARE-/+21} and collagen induced arthritis (CIA)²². In all cases, massive ATX expression was detected in the synovial membrane of inflamed joints almost completely lacking in the control wt joints (FIG. 3A). Moreover, ATX protein levels were found elevated in the plasma of arthritic (hTNF-Tg197) mice (with ELISA assays; FIG. 3B), accompanied by a decrease of its substrate, LPC (measured with LC/MS; FIG. 3C). No other significant differences were detected in the levels of other major phospholipids, although some interesting trends were observed (Supplementary FIG. 3A). No differences in phospholipid levels were detected in joint tissue (normalized according to weight; data not shown), possible due to the inherent heterogeneity (bone and soft tissue) of the joint samples.

### Example 2

### ATX Genetic Ablation from SFs Attenuates mRA

Given the increased ATX expression from SFs in mRA and in order to explore a possible pathogenic role for ATX in disease development, ATX expression was conditionally ablated in SFs utilizing a conditional (LoxP) knock out (cKO) mouse for ATX (Enpp2^{n/n})¹⁷ and a transgenic mouse line expressing the Cre recombinase specifically in SFs (and chondrocytes, dermal fibroblasts) under the control of the ColVI promoter¹⁸.

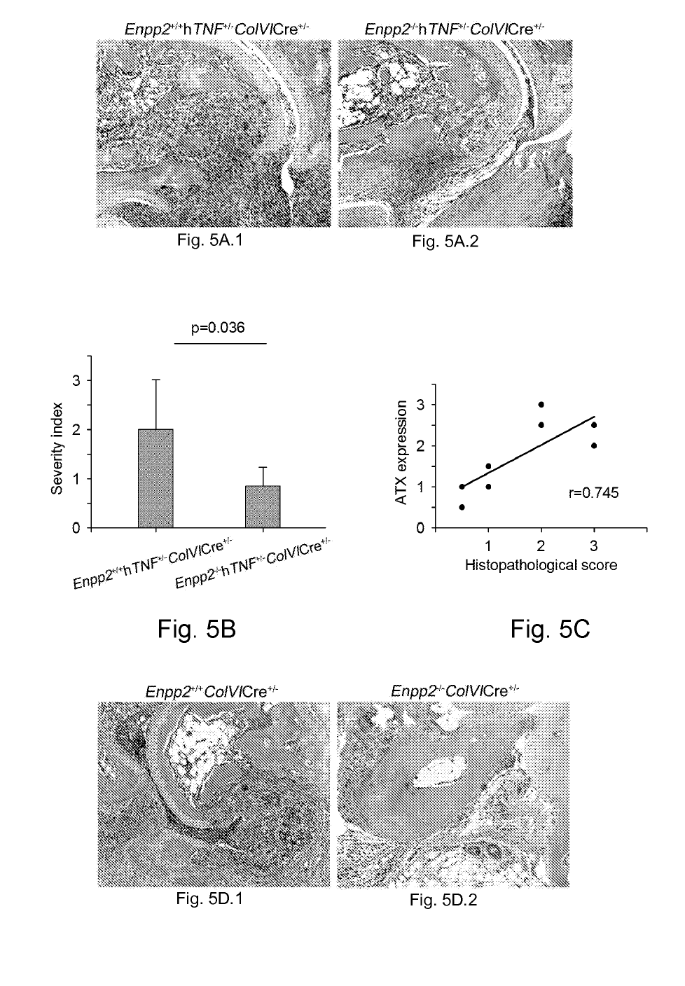

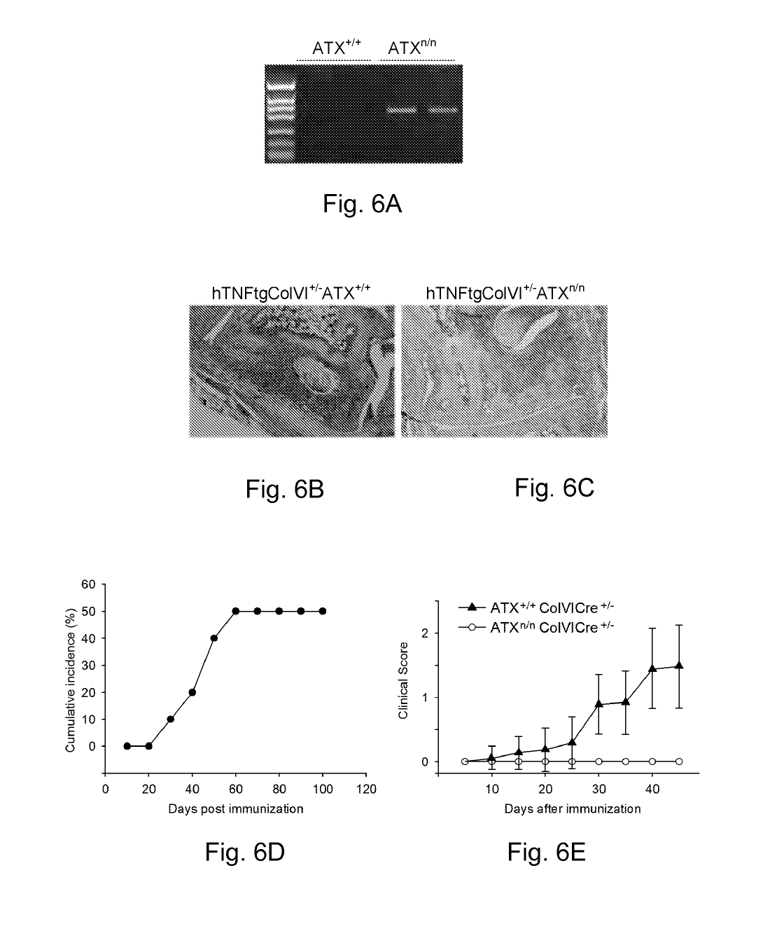

Disease development was then assessed in the hTNF-Tg197 animal model of RA, an inflammatory model driven by the overexpression of hTNF^{19,20}. The lack of ATX SF expression in the joints of Enpp2^{n/n}ColVICre^{+/-}hTNF^{+/-} mice resulted in marked decreased inflammation and synovial hyperplasia, as indicated by the histopathological analysis of the joints (FIG. 5A,B). PCR analysis of DNA extracted from the corresponding paraffin block joint sections indicated correct atx recombination (FIG. 6B), whose efficiency was further evaluated with immunohistochemical staining (FIG. 6C) showing a direct analogy of ATX expression with histopathological score (FIG. 5C).

To confirm a role for ATX in RA development in an autoimmune model, we next investigated the effect of SF-specific ATX genetic ablation on the development of collagen induced arthritis (CIA-H2b)²². As expected for this background²², 50% of wt Enpp2^{+/+}CoVICre^{+/-} mice developed disease symptoms, as opposed to Enpp2^{n/n}ColVICre^{+/-} littermate mice that were completely protected from disease development (Supplementary FIG. 6D). Clinical findings were fully confirmed with histopathological assessment, shown in FIG. 6D.

### Example 8

### Syatemic ATX and LPA Levels and mRA Development

As joint specific ATX expression and most likely local LPA production, was shown necessary for the development of mRA, we next investigated if systemic fluctuations of ATX and LPA would have a pathogenic role in mRA development. To this end, the arthritic hTNF^{+/-} transgenic mice were mated with another transgenic mouse line overexpressing ATX in the liver, driven by the human α1-antitrypsin inhibitor (altl) promoter²³.

### Example 4

### Pharmacological Inhibition of ATX Attenuates the Development of Experimental Arthritis

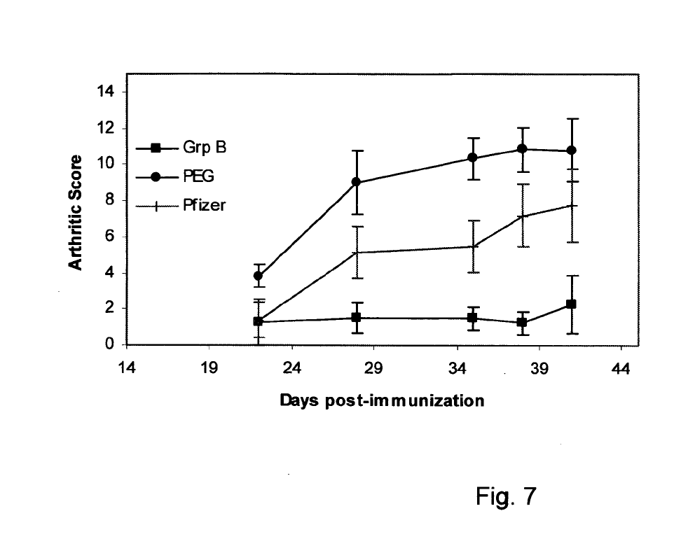

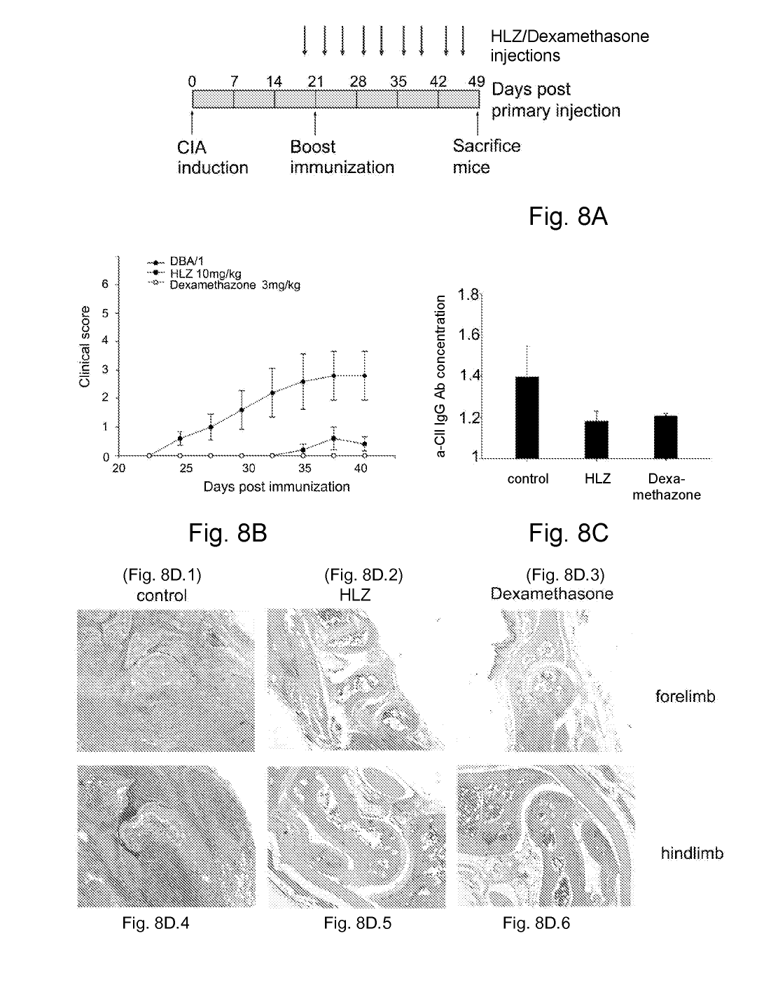

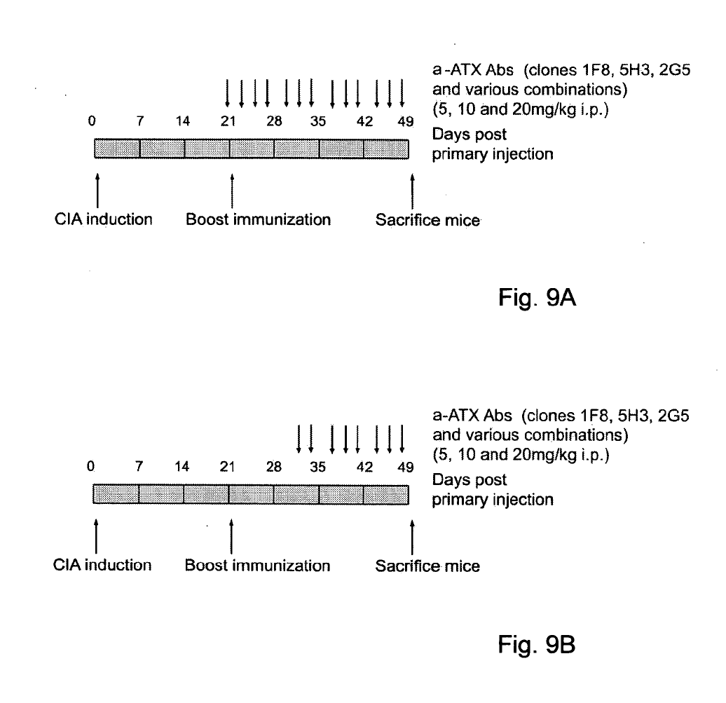

As genetic ablation of ATX from SFs attenuated disease development, we next examined whether pharmacological inhibition of ATX and LPA signaling would also have a similar effect. To this end, we administrated 1-Bromo-3(S)-hydroxy-4-(palmitoyloxy)butyl-phosphonate (BrP-LPA/HLZ), a commercially available (Echelon-inc), dual function pan-antagonist of LPA receptors and inhibitor of the lysophospholipase D activity of ATX^{24,25}. BrP-LPA/HLZ was injected intraperitoneally twice a week as indicated in FIG. 5A. In complete accordance with genetic deletion experiments, inhibition of ATX and LPA signaling attenuated disease development, as indicated by both the clinical and histopathological analysis (FIGS. 8 B and C respectively). On going experiments are examining whether ATX/LPA inhibition would also have a therapeutic effect, by administrating BrP-LPA after disease onset (starting at date 32 post primary collagen injection; FIG. 8A). Furthermore, several more selective inhibitors of either ATX or LPA receptors are already in the pipeline from commercial, collaborative or proprietary sources. Similar results were also obtained with PF-8380 [6-(3-(piperazin-1-yl)propanoyl)-benzo[d]oxazol-2(3H)-one] a specific, potent ATX inhibitor (Gierse, J. K., et al A Novel Autotaxin Inhibitor Reduces Lysophosphatidic Acid Levels in Plasma and the Site of Inflammation. J Pharmacol Exp Ther (2010)), see FIG. 7. We have developed some monoclonal antibodies with good specificity for ATX (table 1, below).

TABLE 1
Characterization of monoclonal antibodies against ATX

| Hybridoma | Isotype | IHC | ELISA | WB |
|---|---|---|---|---|
| 1F8 | IgG1 κ | + | +++ | ++ |
| 2G5 | IgG1 κ | +++ | + | ++ |
| 5H3 | IgG1 κ | +++ | +++ | +++ |
| 7E2 | IgG1 κ | − | + | − |
| 10C10 | IgG1 κ | − | + | +++ |
| 10E2 | IgG1 κ | + | + | ++ |
| 1B1 | IgM λ | − | + | − |

IHC: Immunocytochemistry;
WB: western blot;
IF: immunofluorescence;

### Example 5

### ATX/LPA Stimulate SF Activation and Effector Functions

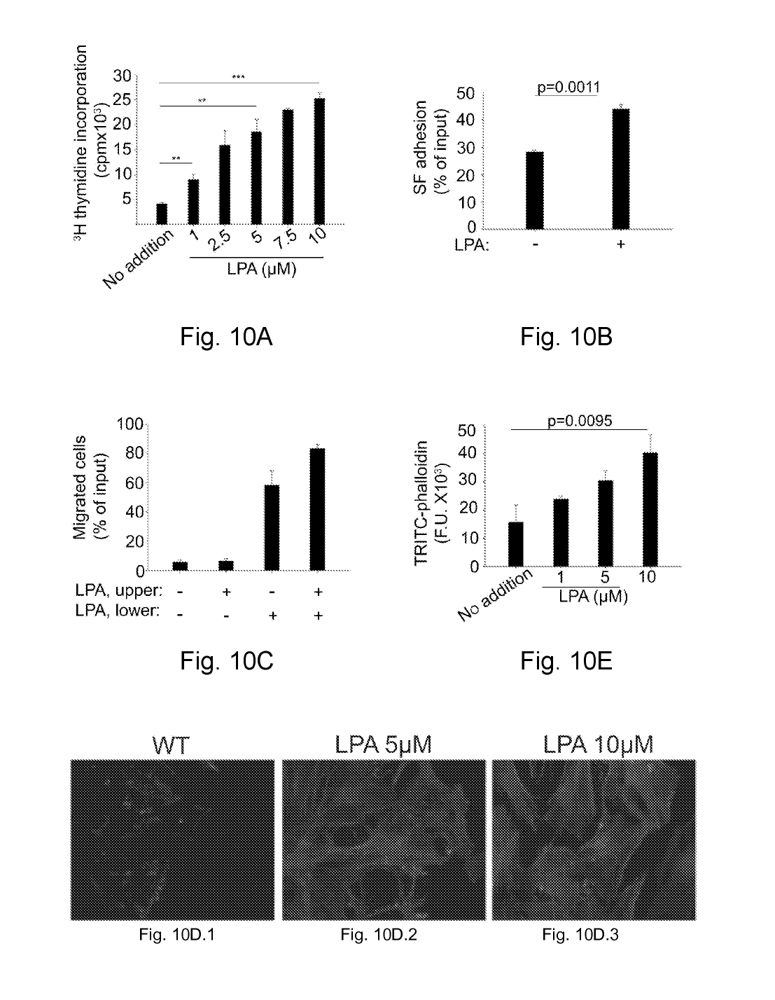

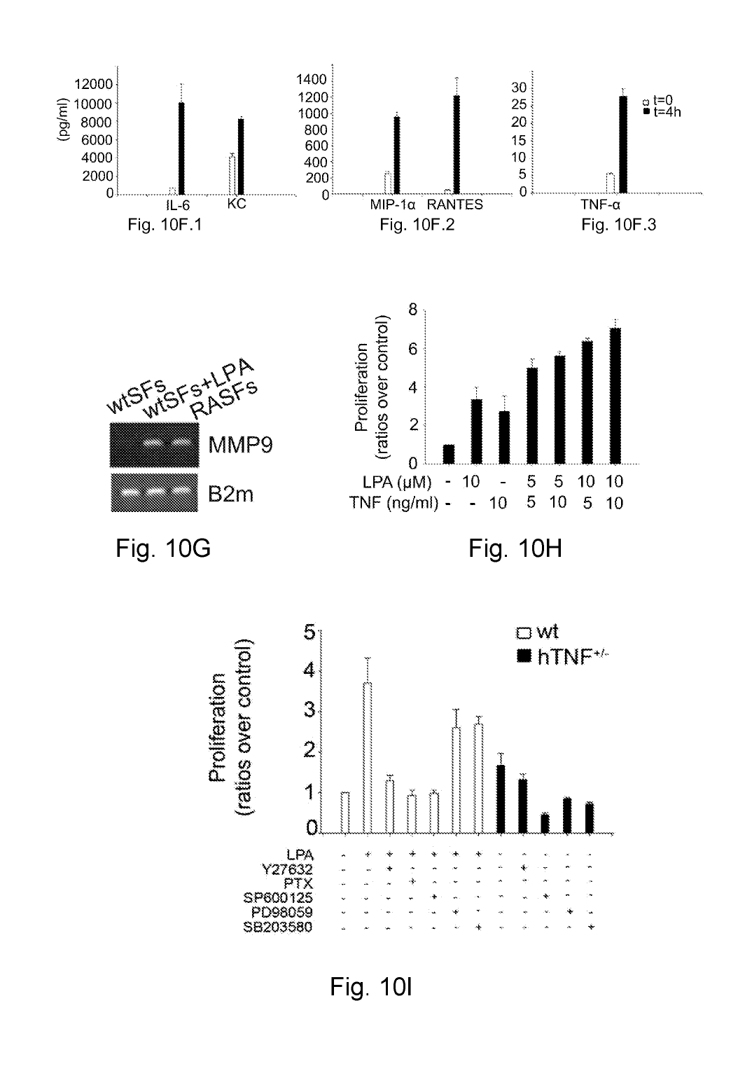

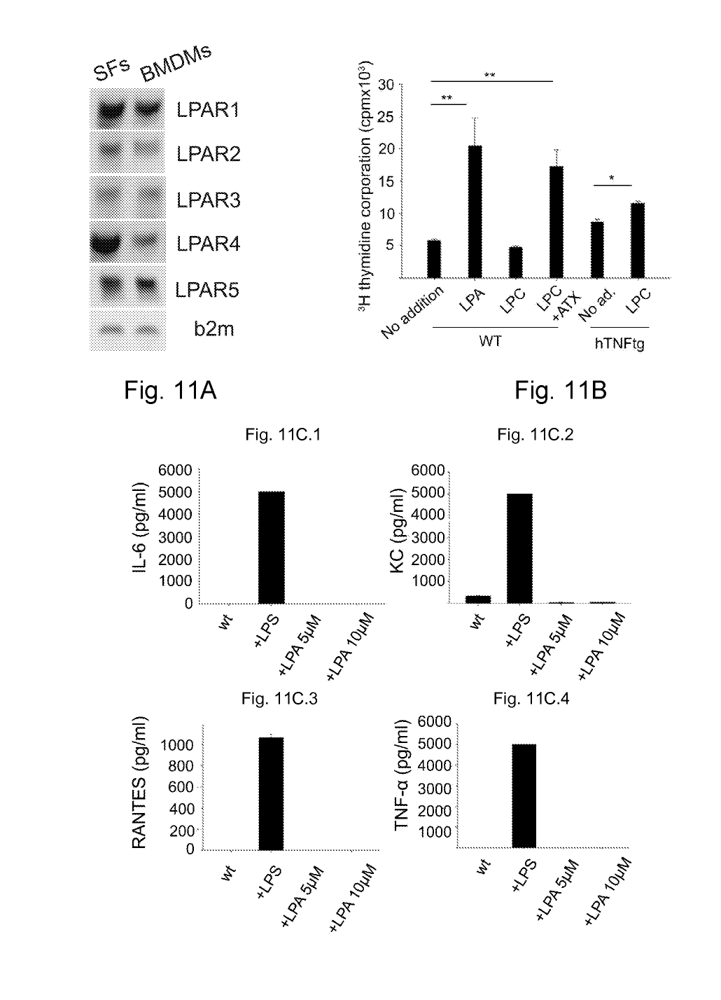

To dissect the mechanistic mode of action of ATX/LPA in synovial cells, a series of in vitro experiments were next performed with isolated mouse wt primary cells. Both SFs as well as BMD macrophages were found, with RT-PCR, to express all the 5 major LPA receptors (LPARs; see FIG. 11A). Nevertheless, addition of LPA in primary cultures while stimulated the proliferation (assessed with ³H-thymidine incorporation) of wt SFs in a dose-dependent manner (FIG. 10A), had no effects in macrophages. LPC, the substrate of ATX, had no effects in wt SF proliferation, unless exogenous ATX was added to convert it to LPA (see FIG. 11B). Accordingly, LPC did stimulate the proliferation of mRA SFs, most likely due to their increased ATX production and consequent LPC hydrolysis to LPA (see FIG. 11B). Moreover, LPA stimulation also resulted to SF increased adhesion and migration (shown with dedicated in vitro assays as previously described^{13,26}; FIGS. 10 B and C respectively), as well as to rearrangements of their actin cytoskeleton (as assessed with phaloidin-F-actin staining; FIG. 10 D,E), all previously reported to be imprinted properties of arthritic SFs¹³. More importantly LPA stimulated proinflammatory cytokine secretion from SFs and especially IL-6 (as accessed with a multiplex Luminex ELISA; FIG. 10F) thus affecting the regulation of the immune system, as well as the production of matrix metaloproteinases (MMPs) (as shown with RT-PCR; FIG. 10G) a hallmark of the RA SF destructive properties. No differences in cytokine secretion from BDM macrophages were observed upon LPA stimulation (FIG. 11C).

As most of the described effects of LPA to SF activation have been also reported for TNF, we next examined if there is a synergy in their mode of action and if they both activate similar intracellular signaling pathways. A concentration dependent synergism in stimulation of proliferation was observed (FIG. 10H), while LPA responses were inhibited not only by an inhibitor of LPA and G-protein signaling (PTX, FIG. 10I), but as well as from inhibitors of ERK, p38, JNK and Rho kinase (PD98059, SB203580, SP600125, Y27632 respectively; FIG. 10I). Inhibitors not only attenuated LPA induced responses of wt SFs, but, as expected, also decreased the proliferation of mRA SFs.

### Example 6

### Increased ATX Expression in Rheumatoid Arthritis

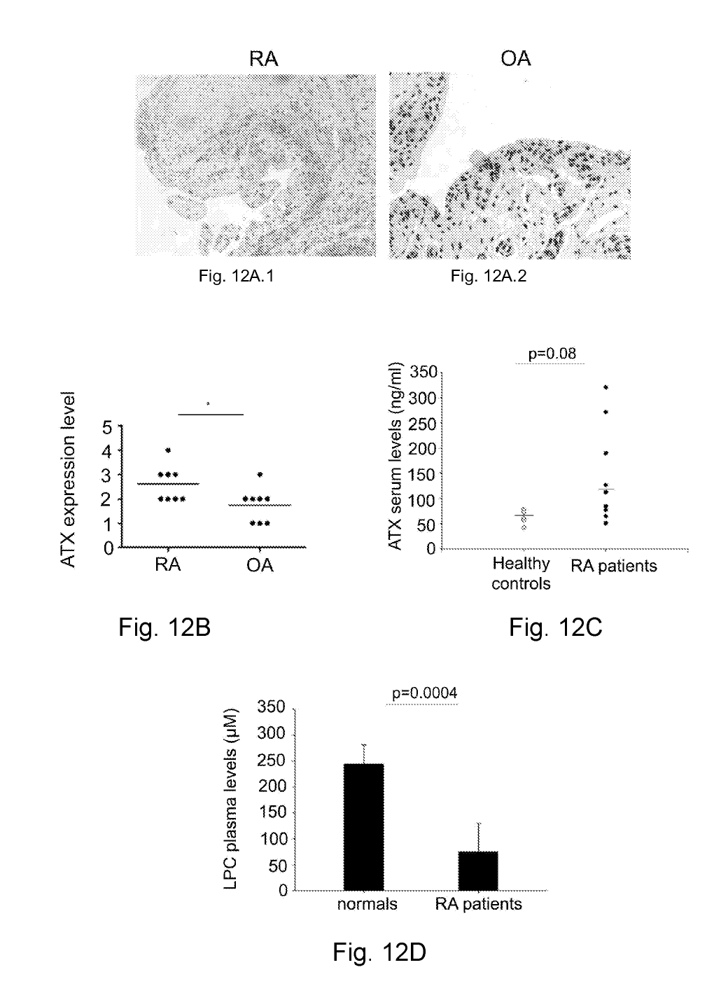

To translate the findings from animal models to the human disease and to examine a possible role for ATX and LPA signaling in rheumatoid arthritis, we examined the expression of ATX in arthritic joints from RA and osteoarthritis (OA, used as negative control) patients (FIG. 12 A). The expression of ATX was significantly higher in RA (FIG. 12 B), primarily detected in activated SFs in the sublining areas and at sites of destruction. As with the animal models, elevated ATX levels were also found in patients sera (with ELISA; FIG. 12 C), accompanied by a decrease of its substrate, LPC, in the corresponding plasmas (with LC/MS; FIG. 12 D). Other major differences were also observed in the phospholipid profile of RA patients (see FIG. 3B on line) in accordance with the increasing interest in phospholipid homeostasis and pathophysiology²⁷.

The following materials and methods were used in Examples 7-8 as needed.

### Mice

All mouse strains used were maintained on a C57Bl/6 genetic background. Animals were housed in the animal facility of the Biomedical Sciences Research Institute “Alexander Fleming” maintained on standard laboratory chow and water ad libitum, and were free of pathogens. All experimentation was approved by an internal Institutional, as well as by the Veterinary service.

### Generation of ATX Mutant Mice

ATX is encoded by Enpp2 gene. We used the Cre-loxP system to generate Enpp2^{+/-} mice carrying a conditional Enpp2 null allele in which exons 1 and 2, containing the transcription initiation sequences of ATX, were flanked by loxP sites and a neo cassette was inserted as a positive selection marker together with the thymidine kinase gene as a negative selection marker. Homozygous mice carrying the recombined targeted allele ATX^{n/n} allele or the ATX^{fl/fl} allele were viable, healthy and fertile. To induce germ line, complete inactivation of ATX, ATX^{fl/fl} mice were mated with transgenic mice overexpressing the Cre recombinase under the control of the human CMV minimal promoter (CMV-CRE).

### Genotyping of ATX Mutant Mice

Genotyping for ATX allele was done by PCR analysis. For PCR analysis, genomic DNA was used as template (cycles of 94° C. for 3 min, of 94° C. for 2 min, 58° C. for 1 min, 72° C. for 1 min, 29 cycles and 72° C. for 5 min) with primers P1 (5′-CGC ATT TGA CAG GAA TTC TT-3′ (SEQ ID NO: 21)), P2 (5′-TAC ACA ACA CAG CCG TCT CA-3′ (SEQ ID NO: 22)), and P3 (5′-ATC AAA ATA CTG GGG CTG CC-3′ (SEQ ID NO: 23)). Expected product sizes for the wild type were 459 bp and for the heterozygote were both 422 bp and 522 bp.

### Immunization

Active EAE in wild-type C57Bl/6 mice was induced as described. Briefly, the mice were challenged in the hind footpads with an emulsion containing 50 μg of MOG p₃₅₋₅₅ peptide ( ) supplemented with 8 mg/ml heat-inactivated Mycobacterium tuberculosis (H37Ra strain; Difco Laboratories) in CFA (Difco Laboratories) on day 0 in the tail base. Booster immunization with an identical emulsion was given on both sides. Bordetella pertussis toxin (PT) (List Biologicals) were administered i.p. (200 ng/mouse) 48 h after the immunization. For induction of EAE in MOGi-cre/Atx^{n/n} mice, the doses for MOG35-55 peptide and PT were the same as for wild type mice Atx^{n/n}. From mice injected at 8 to 12 weeks of age, representative animals were fixed for immunohistochemistry (4% paraformaldehyde in phosphate-buffered saline) and then 7-μm thick frozen sections were used for immunohistochemistry, or fixed in 10% buffered formalin overnight at 4° C. for histopathology. Paraffin sections were prepared using established methods, and then visualized on a Nikon Eclipse microscope (Nikon, Japan) at different disease stages.

### Clinical Assessment

Mice were observed daily for clinical signs of EAE. The severity of EAE was evaluated and scored as follows: 0, no clinical disease; 1, tail paralysis; 2, hindlimb weakness and abnormal gait (incomplete paralysis of one or two hindlimbs); 3, paraplegia (complete paralysis of one or two hindlimbs); 4, quadriplegia; 5, moribund or dead animals.

### Histopathology

Slices of spinal cord were stained by Luxol fast blue staining and nuclear fast red counterstaining for light microscopy (LM) from two or more blocks of tissue from each level. Sections were scored for the degree of demyelination and inflammation.

### Immunohistochemistry

For cryosectioning air-dried sections from spinal cords were fixed in acetone or acetone/methanol (5 minutes each), quenched in 0.3% H2O2/PBS (10 minutes) and after blocking for 1 hour at room temperature in blocking solution 10% heat-inactivated serum (Vector Laboratories,), 0.5% Triton X-100 in TBS (50 mM Tris (pH 7.4), 150 mM NaC) sections were incubated with the primary Abs overnight at 4° C., or for control purposes, PBS. Slides were washed three times for 5 min each in PBS between incubations. Sections were incubated with appropriate secondary antibody conjugated to Alexa fluorophore 488 or 594 (1:500, Molecular Probes, /Invitrogen, Carlsbad, Calif., USA) for 1 h-2 h at room temperature. After incubations positive staining was detected with 3,3′-diaminobenzidine DAB (Vector Laboratories), the slides were mounted and observed under fluorescent microscope. (Nikon Corp., Shinagawa-ku, Tokyo, Japan).

### Neuropathogical Analysis

Experimental spinal cords of mice were paraffin-embedded sectioned and were stained with HE, with Luxol fast blue stain. Immunohistochemistry was performed as previously described. Primary Abs against the following targets was used: autotaxin (anti-ATX from commercially available anti-phospholipase D polyclonal antibody Cat. No. 10005375, Cayman chemical, USA); macrophages (anti-CD11b;) and glial fibrillary acidic protein (GFAP) for astrocytes (Dako, Carpinteria, Calif.). Blocks of lumbar spinal cord embedded in optimal cooling temperature medium (Tissue-Tek, Sakura Finetek, Torrance, Calif.), were cut as 8 μm sections. The extent of inflammation was quantified by counting the perivascular inflammatory infiltrates in 5 randomly selected sections of lumbar spinal cord. The size of demyelinated lesions was determined by overlaying the spinal cord sections with a morphometric grid and counting the area of demyelination in relation to the total area of the spinal cord.

### Microscopy

Stained sections were examined and photographed using a light microscope (Nikon Corp., Shinagawa-ku, Tokyo, Japan) or a fluorescence microscope (Nikon Corp., Shinagawa-ku, Tokyo, Japan) connected to a camera (, Olympus). Digital images were collected and analyzed using camera software. Images were assembled using Adobe Photoshop (Adobe Systems, San Jose, Calif.).

### Example 7

### Increased ATX Expression in Demyelinated Regions In an Animal Model of MS

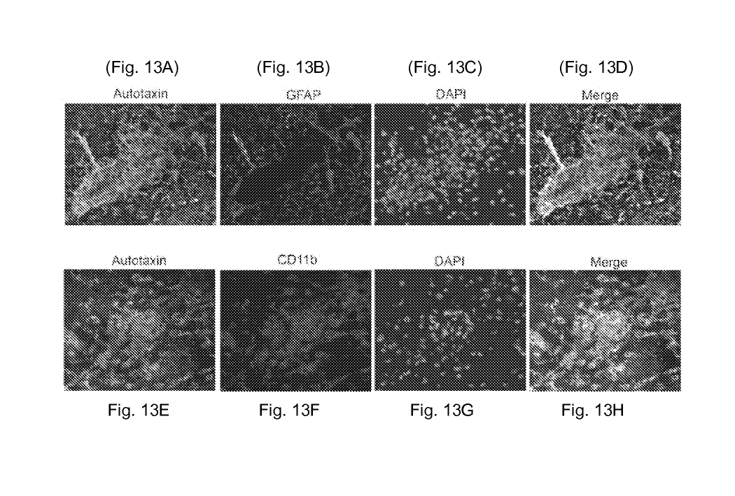

Significant ATX expression was detected in the cerebrospinal fluid of patients suffering with Multiple Sclerosis (MS), completely lacking from the control samples⁴⁶, suggesting a role for ATX in maintenance of cerebrospinal fluid homeostasis during pathological/demyelinating conditions⁴⁷. To explore a possible role of ATX and LPA signalling in the pathophysiology of Multiple Sclerosis we first examined ATX expression in an animal model of MS, Experimental autoimmune encephalomyelitis (EAE). The disease was induced upon injection-immunization with 50 μg of MOG peptide essentially as previous reported⁶⁴. Spinal cord paraffin sections were then stained with an a-ATX antibody, as well as with CD11b (macrophages, microglia) and GFAP (astrocytes) antibodies. As it can be seen in FIGS. 13A-13H, ATX expression was found overexpressed in EAE, in demyelinated areas, colocalized with inflammatory cells. Some co-localization with activated astrocytes (GFAP⁺) could also be detected as reported previously9.

### Example 8A

### Genetic Ablation of ATX from Oligodendrocytes Attenuates the Development of EAE

ATX mRNA was identified as highly upregulated during oligodendrocyte (ODC) differentiation³⁵ and ATX protein expression is also apparent in maturing ODCs, temporally correlated with the process of myelination^{28,36}. Therefore and in order to verify the role of ATX in MS/EAE pathophysiology, ATX was genetically ablated in ODCs by mating the proprietary conditional knock out (LoxP) for ATX⁶⁵ with a knock in mouse strain expressing the Cre recombinase under the control of the ODC-specific myelin ojigodendrocytes glycoprotein (MOG) promoter (MOGi-Cre)⁶⁴. Normal mendelian ratios were observed in litters, indicating that ATX ablation in oligodendrocytes is not lethal. Moreover ATX^{n/n}MOG-Cre^{+/-} appeared (gross) morphological normal, indicating proper development.

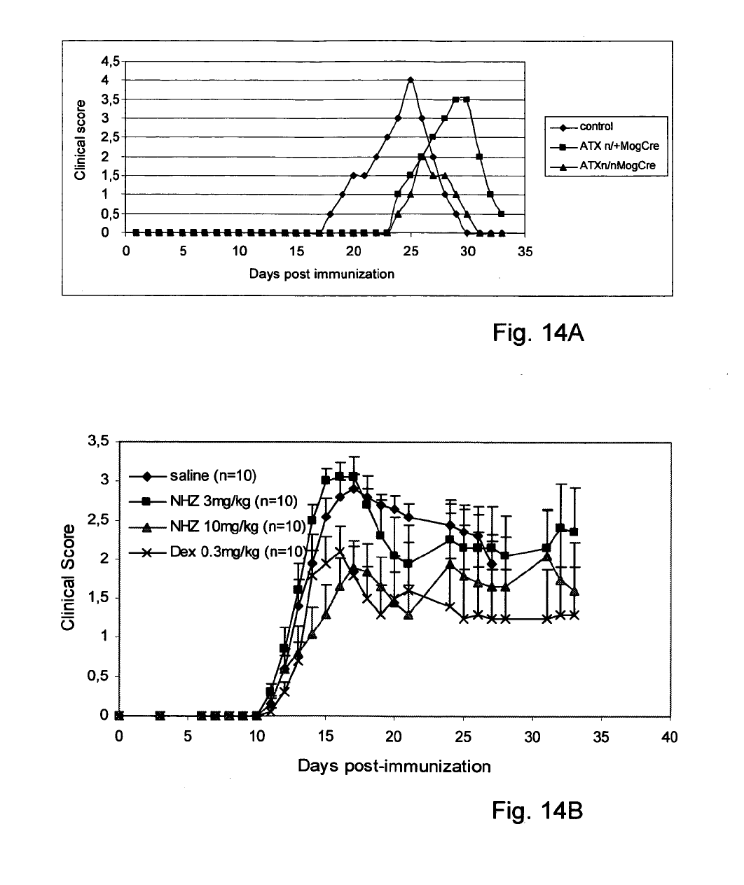

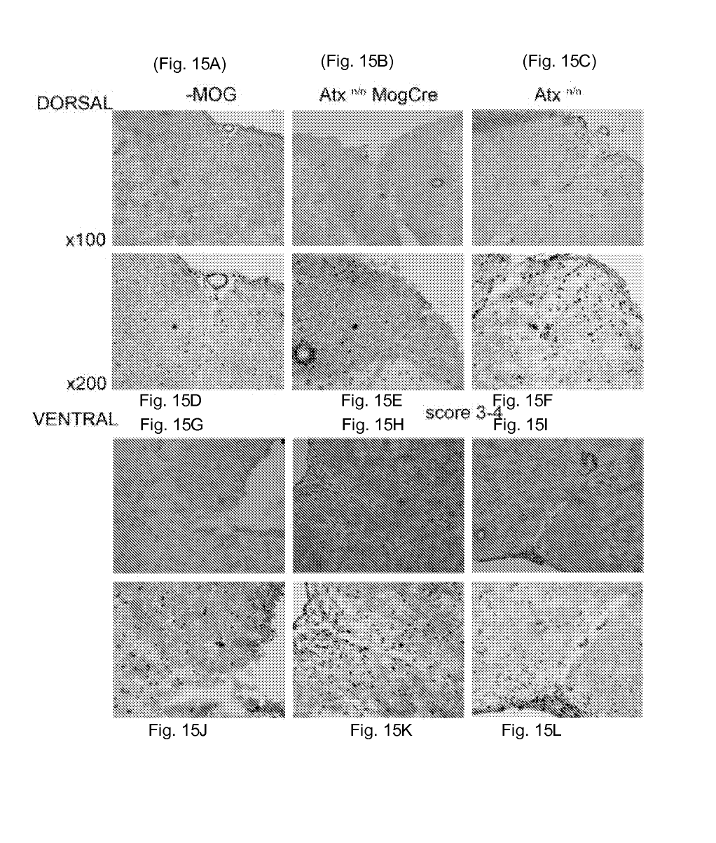

ATX^{n/n}MOG-Cre^{+/-}, ATX^{n/+}MOG-Cre^{+/-} and control ATX^{n/n} mice were then injected/immunised with the MOG peptide essentially as previous reported⁶⁴. 100% of control ATX^{n/n} mice developed EAE with an onset at 16 days p.i., reaching a clinical score of 4 (FIG. 14A), as expected⁶⁴ and as described in Materials and Methods. In contrast, onset of disease in the ATX^{n/n}MOG-Cre^{+/-}, was observed at 24 days p.i. reaching a clinical score of 2 (FIG. 14A). Intermediate results were obtained with heterozygous KOs (FIG. 14A). Histological analysis was performed to assess whether the reduction of clinical disease severity correlates with reduced demyelination and/or inflammation. CNS sections were stained with H & E to quantify inflammation and with Luxol fast blue to determine the extent of demyelination. As shown in FIG. 3, there is a clear reduction of inflammation and demyelination in the ATX^{n/n}MOG-Cre^{+/-} mice compared with controls, consistent with the reduced clinical score.

Therefore genetic deletion of ATX from oligodendrocytes resulted in a significant delay of disease onset and attenuation of disease severity. On going pharmacological studies (small molecules and a-ATX antibodies) are expected to validate ATX as a therapeutic target in MS.

### Example 8B

### Pharmacological Inhibition of ATX Attenuates the Development of EAE

As genetic ablation of ATX from ODCs attenuated disease development, we next examined whether pharmacological inhibition of ATX and LPA signaling would also have a similar effect. To this end, we administrated 1-Bromo-3(S)-hydroxy-4-(palmitoyloxy)butyl-phosphonate (BrP-LPA/HLZ), a commercially available (Echelon-inc), dual function pan-antagonist of LPA receptors and inhibitor of the lysophospholipase D activity of ATX (Xu, X. & Prestwich, G. D.

Inhibition of tumor growth and angiogenesis by a lysophosphatidic acid antagonist in an engineered three-dimensional lung cancer xenograft model. Cancer 116, 1739-1750 (2010); Xu, X., Yang, G., Zhang, H. & Prestwich, G. D. Evaluating dual activity LPA receptor pan-antagonist/autotaxin inhibitors as anti-cancer agents in vivo using engineered human tumors. Prostaglandins Other Lipid Mediat (2009)).

BrP-LPA/HLZ was injected intraperitoneally twice a week at 10 mg/Kg. In accordance with genetic deletion experiments, inhibition of ATX and LPA signaling attenuated disease development, as indicated by the clinical score (see FIG. 14B).

The following materials and methods were used in Examples 9-11 as needed.

### B3.3 Materials and methods

Human patients.

65 newly diagnosed IIP patients with idiopathic interstitial pneumonias of four different histopathologic patterns (Table 2) were recruited in our study. The diagnosis of IIPs was based on the consensus statement of the ATS/ERS in 2002⁷⁰. Subjects were separated according to the histopathologic pattern of the IIPs as shown in Table 2. All patients were treatment naive when included in the study. Paraffin-embedded surgical lung specimens (open lung biopsy or by video assisted thoracoscopic surgery-VATS) from three different fibrotic regions of each individual were sampled. All patients, following protocol approval by the local ethics committee (#1669), signed an informed consent form where they agreed to the anonymous usage of their lung samples for research purposes.

TABLE 2
Demographic and spirometric characteristics of IIP patients
(IPF/usual interstitial pneumonia, IPF/UIP; cryptogenic organizing
pneumonia/organizing pneumonia, COP/OP; fibrotic non specific
interstitial pneumonia, fNSIP; cellular NSIP, cNSIP) and control subjects.
Values are expressed as mean + SD, and age as median (range).

| Characteristics | IPF/UIP | COP/OP | NSIP | Control |
|---|---|---|---|---|
| Number | 25 | 20 | 20 | 20 |
| Sex: Male/Female | 18/7 | 12/8 | 11/9 | 10/10 |
| Age, median (yr) | 65 (43-72) | 50 (38-62) | 60 (41-68) | 36 (29-60) |
| Smokers/non | 21/4 | 7/13 | 12/8 | 13/7 |
| smokers | | | | |
| FVC, % pred | 66 ± 3* | 78 ± 4* | 71 ± 3* | 101 ± 12 |
| TLC, % pred | 58 ± 4* | 77 ± 3* | 69 ± 4* | 99 ± 11 |
| DL_{CO} % pred | 48 ± 6* | 68 ± 5* | 61 ± 4* | 88 ± 6 |

FVC: Forced Vital Capacity;
DLco: Diffusion Lung capacity of carbon monoxide;
TLC: Total Lung Capacity;
*denotes statistical significance (p < 0.001).

### Animals

All mice were bred at the animal facilities of the Alexander Fleming Biomedical Sciences Research Center, under specific pathogen-free conditions. Mice were housed at 20-22° C., 55±5% humidity. and a 12-h light-dark cycle; water and food was given ad libitum. CC10Cre^{+/-} mice were kept in a mixed CBA/C57.Bl6 background while Enpp2^{n/n} mice were bred on a C57.Bl6 background. Cohorts of CC10Cre^{+/-}/Enpp2^{n/n} (hereafter CC10Enpp2^{n/n}) and their Enpp2^{n/n} littermates were generated by crossing CC10Cre^{+/-}Enpp2^{n/n} mice with Enpp2^{n/n} mice.

### BLM Animal Model

8-12 week old littermate mice were divided into an experimental group that received intratracheal Bleomycin (BLM, 0.08 U) and a control group that received Normal Saline instead. Mice were sacrificed at 7 and 14 days post BLM instillation for pathology evaluation.

### Inhibition Studies

Age and sex-matched 6-8 week old WT (C57/Bl6) mice were divided in four study groups according to the treatment regime.

Group A (n=5) received i.p injections of Veh (water) twice per week for two weeks starting at day −1 and instilled with Saline intratracheally at day 0.

Group B (n=3) received i.p injections of BRP-LPA (10 mg/Kg) twice per week for two weeks starting at day −1 and instilled with Saline intratracheally at day 0.

Group C (n=9) received i.p injections of Veh twice per week for two weeks starting at day −1 and instilled with BLM (0.08 U) intratracheally at day 0.

Group D (n=8) received i.p injections of BRP-LPA (10 mg/Kg) twice per week for two weeks starting at day −1 and instilled with BLM (0.08 U) intratracheally at day 0. A schematic representation of the time course of the protocol is depicted below.

All mice were sacrificed at day 14 after BLM instillation for disease evaluation. Blood was drawn from the inferior vena cava and added in 50 mM EDTA for plasma preparations. Lungs were lavaged 3 times with 1 ml aliquots of saline (BAL) followed by perfusion through the right ventricle of the heart with 10 ml of PBS. BAL fluid was centrifuged and cell pellets were used for total cell counts with Trypan Blue while the supernatant was snap frozen.

Right lung was instilled with 1 ml of buffered formalin, dissected and fixed O/N in formalin. Left lung was snap frozen and used for soluble collagen measurements using the Sircol assay.

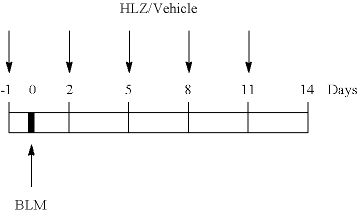

Schematic representation of BRP-LPA administration protocol. BRP-LPA (or water) was administered intraperioneally in mice starting one day before BLM (or Sal) instillation and every three other days until the end of the study (14 days). Pulmonary fibrosis was induced with a single i.t. instillation of BLM at day 0.

### Histology and Immunocytochemistry

Upon euthanasia, blood was drawn from the vena cava and was added to 50 mM EDTA for plasma preparations. Lungs were lavaged with an aliquot of 1 ml normal saline followed by 2 ml of saline (Bronchoalveolar Lavage Fluid, BALF). BALF aliquots were centrifuged; the cell pellets were combined and used for total cell counts with Trypan Blue while supernatants were snap-frozen. Lungs were then perfused with 10 ml of PBS through the spontaneous beating right ventricle and the left lung was snap-frozen for future analysis while the right lung was fixed in 10% neutral buffered formalin. Tissues were embedded in paraffin, and 5-μm-thick sections were cut at the median transverse level of the lungs. Sections were mounted on glass slides and stained with H&E.

### Immunohistochemistry

Immunostaining was performed with peroxidase labeling techniques. Tissue sections were deparaffinized, and endogenous peroxidase activity was blocked by incubation in 1% peroxide. The sections were preincubated with 2% FBS in PBS-Tween for 30 minutes, followed by incubation overnight with the primary antibody. Sections were then washed in PBS-T and incubated for 30 minutes with horseradish peroxidase (HRP)-conjugated anti-rabbit IgG (1:1000 dilution in PBS-T) or with. The sections were further washed with PBS-T. Finally, color was developed by immersing the sections in a solution of 0.05% 3,3′-diaminobenzidine (DAB; Sigma) and 0.01% hydrogen peroxide in PBS.

The sections were counterstained with hematoxylin. For immunofluorescence, sections treated as above were incubated for 30 min with AlexaFluor-488 or -555 conjugated secondary antibodies. Finally, sections were further stained with DAPI for 5 min before mounting.

### ATX Activity Assay

Autotaxin activity in BALF of mice was determined using the cleavage of the ATX-specific fluorogenic substrate FS-3 (Echelon Biosciences, UT, USA). Assays were conducted in a final volume of 100 μl comprising 20 μl 5× assay buffer (700 mM NaCl, 25 mM KCl, 5 mM MgCl₂ and 250 mM Tris, pH 8.0), 20 μl of FS-3 (final concentration 2.5 μM) and 60 μl of BALF (diluted 1:2 in 1×PBS). Reactions were incubated at 37° C. in a Tecan Infinite 200 microplate reader (Tecan Trading AG, Switzerland) set to make fluorescence measurements every 5 min for 1 hour. Autotaxin activity was quantitated by measuring the rate of increase in fluorescence at 528 nm with excitation at 485 nm.

### Total Protein Content Determination

Total protein concentration in BALFs was measured using the Bradford protein assay (Biorad, Hercules, Calif.) according to manufacturer's recommendations. OD readings of samples were converted to μg/ml using values obtained from a standard curve generated with serial dilutions of bovine serum albumin (50-500 μg/ml).

### Sircol Assay

Soluble collagen measurements were performed using the Sircol reagent according to manufacture's recommendations.

### ATX ELISA

To detect ATX in biological fluids, a direct ELISA assay was developed using the commercially available anti-phospholipase D polyclonal antibody (Cat. No. 10005375, Cayman chemical, USA). The bottom of a NUNC-IMMUNO 96 MicroWell Elisa plate was coated overnight with 100 μl of 1:200 diluted serum in 0.05 M carbonate/bicarbonate coating buffer, pH 9.5 (Cat. No. S7795/S7277, Sigma, Saint Louis, Mich., USA), washed three times with 0.05% Tween-20 (Cat. No. P1379, Sigma, USA) in TBS and blocked with 0.1% Bovine Serum Albumin (Cat. No. A7888, Sigma, USA) in 0.05% TBST for 1 h at RT. For each ELISA assay, 100 μl of two-fold serial dilutions of recombinant ATX protein ranging from 0.1 to 1.6 μg/ml were plated as standard curve and treated as all samples.

The plate was then incubated with 0.5 μg/ml detection antibody for 1 h and washed three times with 0.05% TBST. Autotaxin antigen was then detected with an anti-rabbit HRP-labeled secondary antibody (Cat. No. 4010-05, SouthernBiotech, USA) that was developed with TMB substrate (Cat. No. A7888, Sigma, USA). Readings were obtained at 450 nm. All samples and standards were assayed in triplicates.

### Example 9

### Increased ATX Expression in Pulmonary Fibrosis

It has been recently demonstrated that LPA through signaling from LPR1 mediates key aspects of the fibrogenic response such as fibroblast recruitment and vascular leak in the BLM animal model of the disease. Furthermore, increased levels of LPA were also measured in BAL samples of IPF patients and inhibition of LPA signaling markedly reduced fibroblast responses to IPF BAL chemoattractant activity⁶⁶. Since the biologic activity of LPA is mainly regulated by its concentration we sought to determine the expression levels of ATX, the enzyme largely responsible for the generation of LPA in the extracellular space.

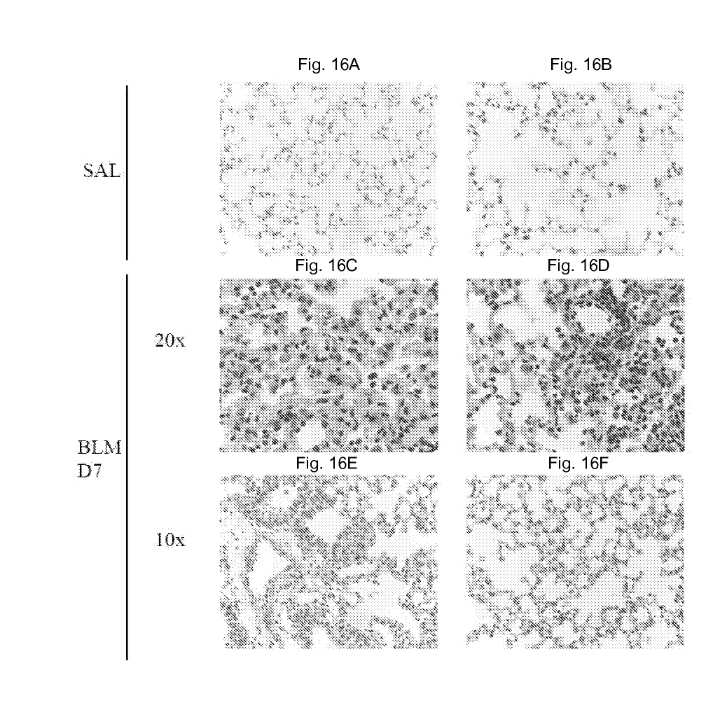

ATX expression was assessed both in the bleomycin-induced animal model of pulmonary fibrosis, as well as in lung sections of human Idiopathic pulmonary fibrosis (IPF) and the corresponding controls. As shown in FIG. 16, ATX expression was found significantly upregulated upon BLM injection and the development of pulmonary fibrosis.

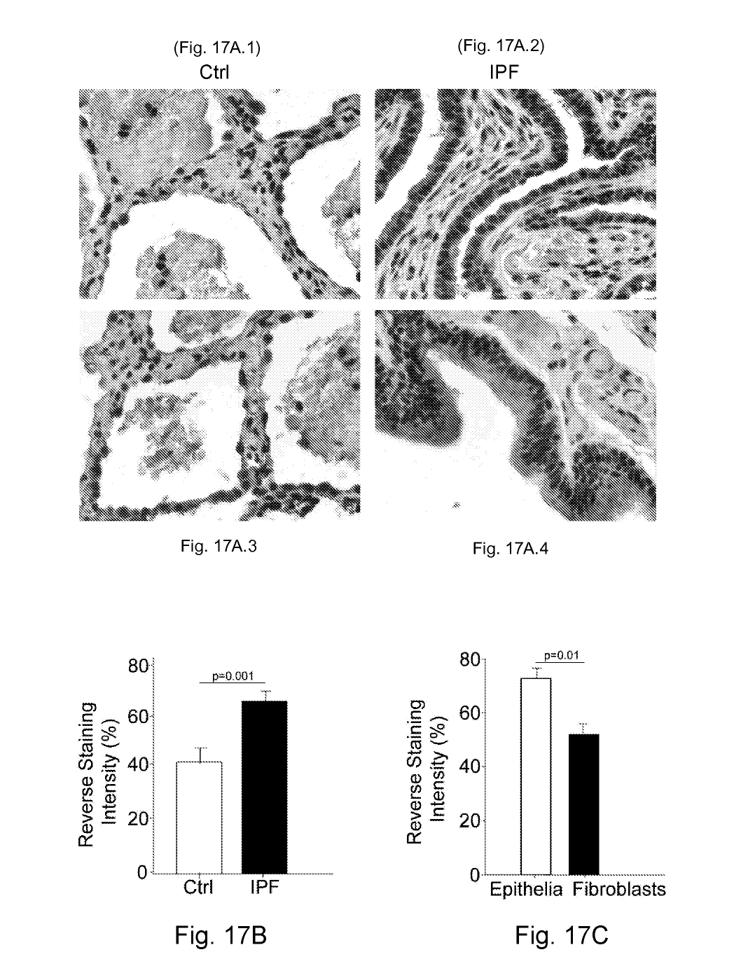

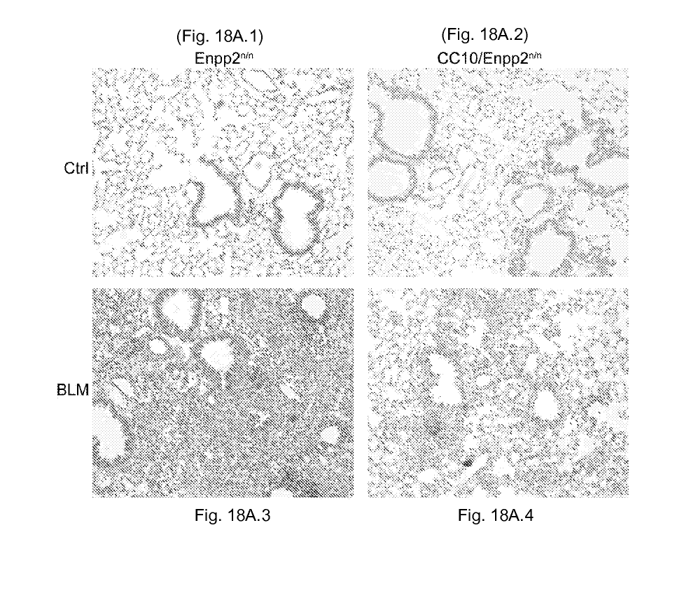

As shown in FIG. 17A, significant expression of ATX was also observed in IPF/UIP almost missing from normal lung control samples. ATX exhibited strong positive staining within areas of the epithelium (bronchial and alveolar) and to a lesser extent in adjacent fibroblastic foci, within the IPF lung. Computational and statistical analysis of signal intensities of all 85 samples revealed an overall increase of ATX expression in IPF/UIP samples as opposed to normal samples (FIG. 17B) and further quantified the differences in signal intensities that were noted between epithelial and fibrotic areas of IPF lung (FIG. 17C). These results indicate that the increased LPA levels in IPF BAL that have been previously reported could be the outcome of ATX's action on available phospholipid precursors such as LPC which is known to be abundant in biological fluids such as serum⁶⁷ and BAL (at least in mice).

### Example 10

### Genetic Deletion of ATX from Bronchiolar Epithelium Attenuates the Development of BLM-Induced Pulmonary Fibrosis

In order to confirm a possible role of ATX in the development of BLM-induced pulmonary fibrosis we genetically ablated the Enpp2 gene specifically in CC10⁺ (Clara) cells of the bronchiolar epithelium by means of Cre mediated recombination. Immunohistochemical studies in lung sections of naïve WT mice indicated that ATX is mainly expressed by airway epithelial cells and to a lesser extent by alveolar epithelial cells (FIG. 16). Therefore we used the CC10/Cre mouse strain in which the Cre cDNA was placed under the control of the rat CC10 promoter driving its expression specifically in CC10⁺ cells of the bronchi. Mating of the conditional Enpp2^{n/n} mice with CC10/Cre mice resulted in the generation of CC10/Cre-Enpp2^{n/n} mice (CC10/Enpp2), in which the Enpp2 gene is sufficiently deleted in bronchiolar epithelial cells. BLM administration in WT mice as expected resulted in progressive subpleural/peribronchial inflammatory changes and by day 14 in the development of fibrotic areas that were diffusely distributed throughout lung parenchyma. Histological examination of mouse lungs revealed significant damage to the lung tissue, including thickening of the alveolar septae, alveolar inflammation and fibrous obliteration of the peribronchiolar and parenchymal regions (FIG. 18A, Enpp2^{n/n}). In contrast, CC10Enpp2 mice following BLM instillation displayed less pathological features with focal areas of fibrosis development that were restricted mainly to peribronchial spaces (FIG. 18A, CC10Enpp2^{n/n}). Concomitantly, soluble collagen determination at day 14 verified the histological observations, as CC10Enpp2 mice displayed statistically significant reduction in soluble collagen accumulation when compared to their WT littermates (FIG. 18B). Furthermore BAL total cell counts, an index of BLM-induced inflammation, were gradually elevated at day 7 and peaked at day 14 post BLM administration in WT mice; an effect that was attenuated in the case of CC10Enapp2 mice in which total cellularity was significantly reduced at day 14 (FIG. 18C). In order to examine whether BLM treatment affects ATX expression in airspaces, we quantified BAL ATX levels with a corresponding ELISA assay. As shown in FIG. 18D, ATX was progressively upregulated in BALF of WT mice and peaked at day 14, when fibrosis development was maximal. CC10Enpp2 mice exhibited moderate increases in ATX levels that reached statistical significant difference compared to WT mice at day 14 (FIG. 18D). ATX levels were also measured in serum of both mouse strains but as can be seen in FIG. 18E no differences were noted between them or between Ctrl and BLM-treated groups.

In conclusion, these studies revealed that conditional deletion of the Enpp2 gene from bronchiolar epithelial cells (Clara cells) resulted in attenuation of fibrosis development in the relevant BLM animal model. They also revealed that BLM administration induces ATX expression in the lungs of mice an observation that could explain how LPA levels increase in the course of BLM-induced pulmonary fibrosis. Moreover the fact that deletion of ATX from bronchial epithelial cells in mice failed to adequately increase BAL ATX's levels after BLM instillation suggests that these types of lung epithelial cells, at least in pathological states, may constitute one of the major contributors to the elevated ATX levels. Finally, the increased expression of ATX in IPF/UIP lungs fits perfectly into a scheme in which pathological raise of its production and activity could explain the previously reported increased concentrations of LPA in the BAL of IPF patients.

### Example 11

### Pharmacologic Inhibition of ATX Attenuates the Development of BLM-Induced Pulmonary Fibrosis

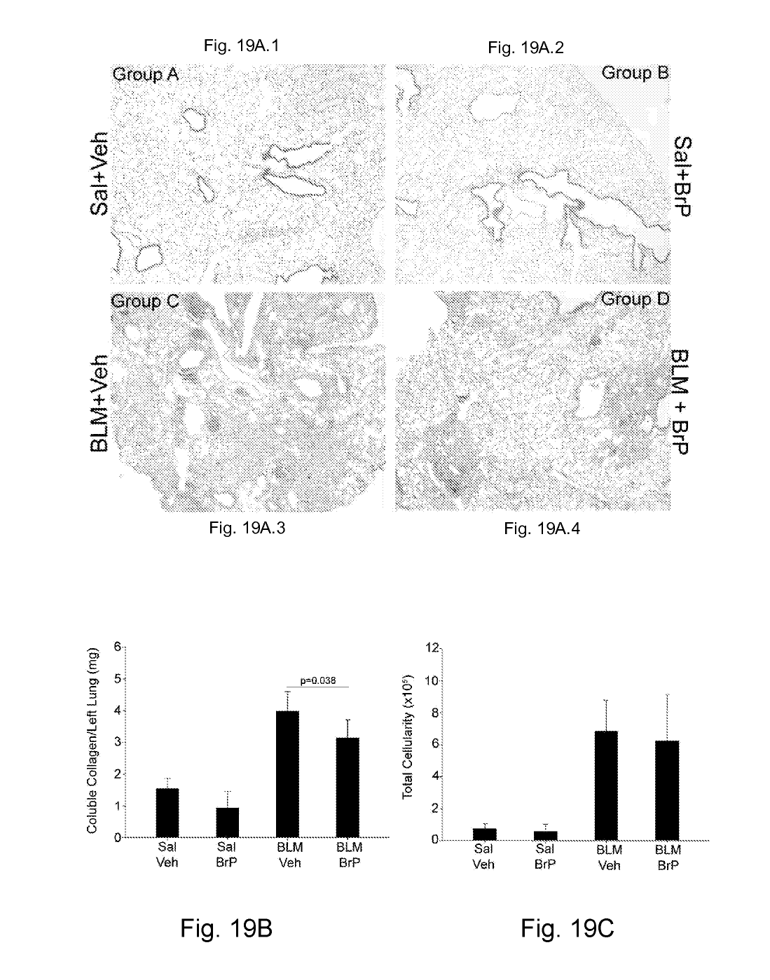

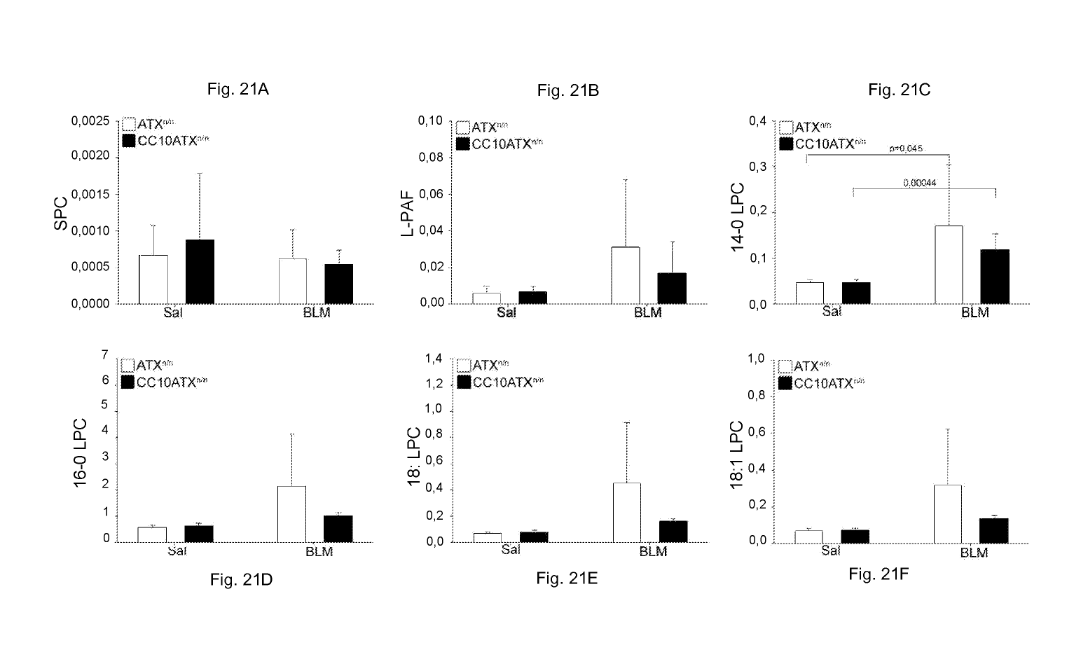

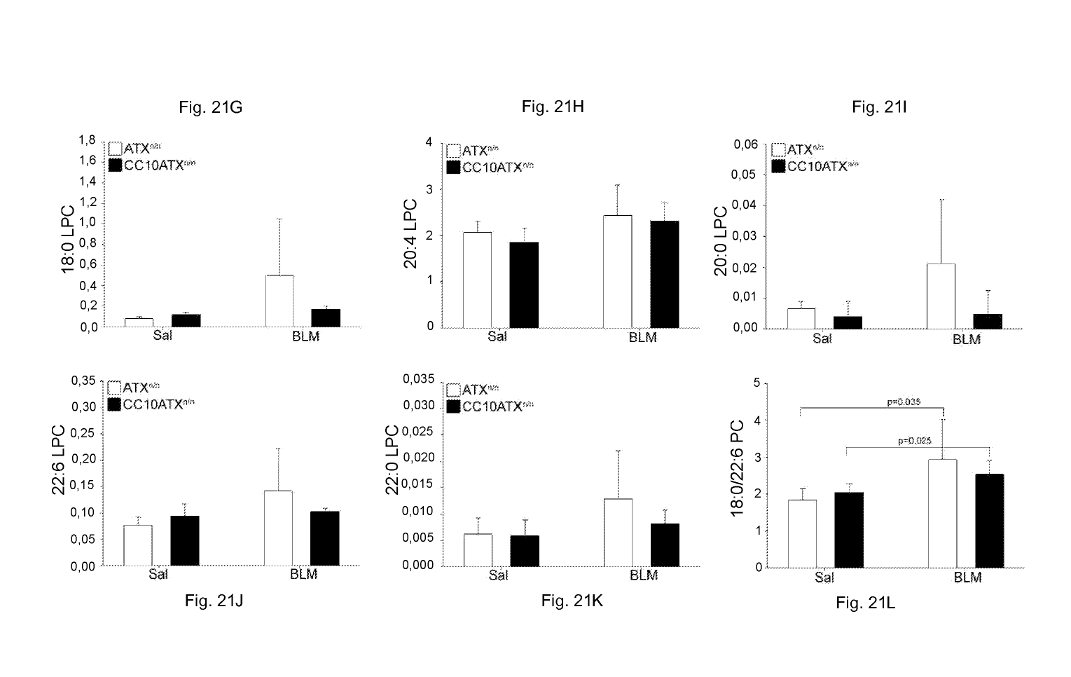

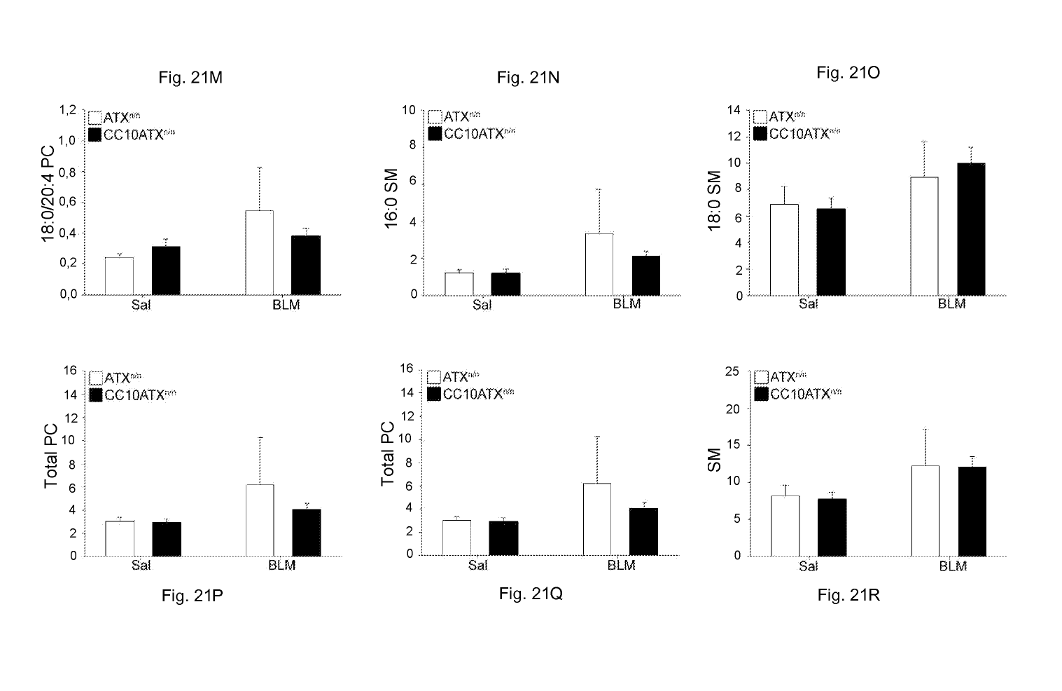

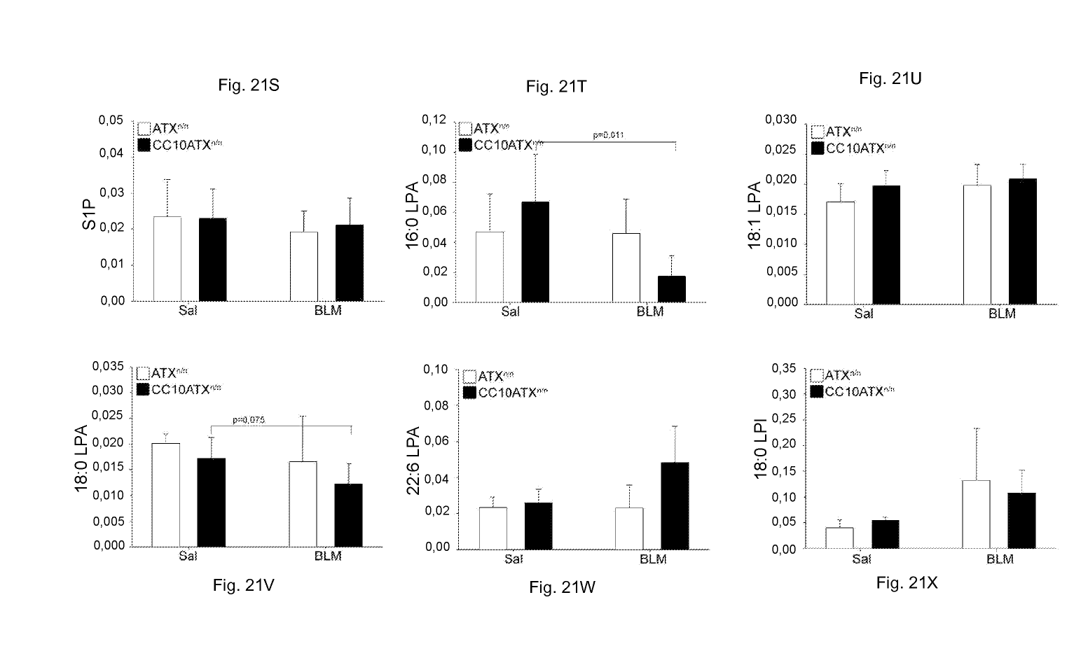

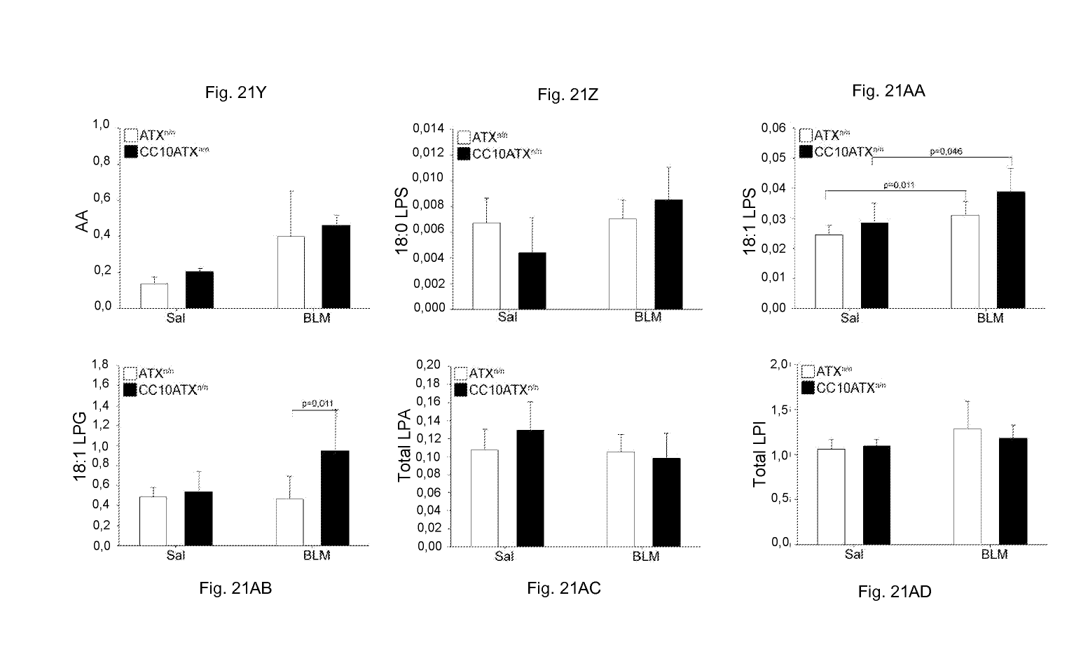

In order to explore for a possible therapeutic use of ATX inhibition in the context of BLM-induced lung pathology, we investigated the effects of the administration of a specific inhibitor of both ATX and LPA receptors, BrP-LPA, in the development of BLM-induced pulmonary fibrosis^{68,69}. To this end, WT mice (C57/BL6) were administrated with biweekly i.p. injections of BrP-LPA (10 mg/Kg), as depicted schematically in FIG. 21, while another cohort of mice was injected with vehicle alone and served as the control group. Remarkably, ATX/LPAR inhibition resulted in similar phenotypes to the genetic inhibition of Enpp2 regarding fibrosis development. Specifically, ATX/LPAR inhibition resulted in the development of focal areas of fibrosis with a patchy distribution in lung parenchyma, as opposed to the vehicle-treated group that manifested a more diffuse pattern of fibrosis, obliterating larger areas of lung tissue (FIG. 19A).

These observed histopathological differences were also reflected in soluble collagen measurements, as Brp treatment had as a result the significant reduction of collagen accumulation when compared to Veh-treated mice (FIG. 19B). In agreement with previous reports⁶⁶ and contrary to the genetic studies described above, BAL total cell counts did not differ between Brp- and Veh-treated groups 14 days post BLM administration (FIG. 19C).

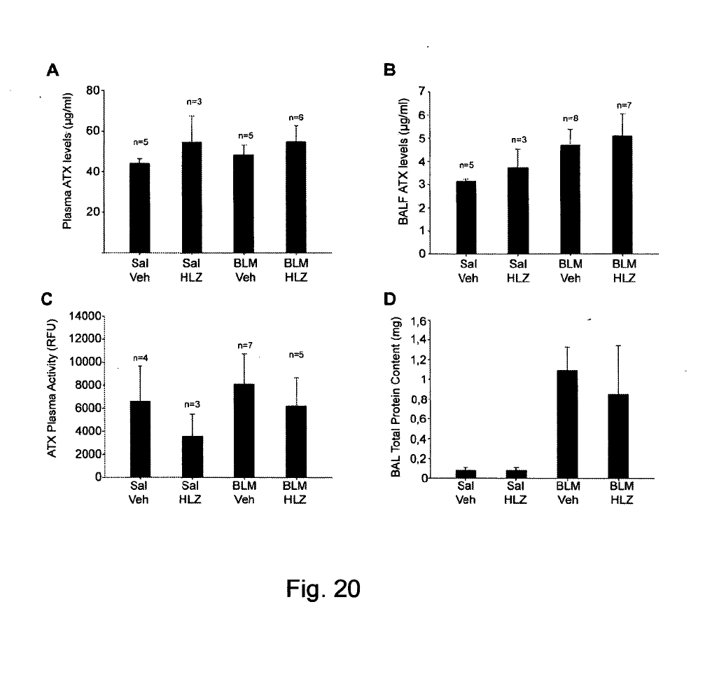

In order to investigate the effects of BrP-LPA treatment in ATX concentration and activity, we performed ELISA assays in plasma and BAL fluids obtained from control and BLM treated mice. As shown in FIG. 20, BrP treatment led to a non statistical increase in the levels of ATX both in plasma and in BAL fluids of saline treated, naïve mice (FIG. 20A, B). The same trend of ATX regulation was also noted in biological fluids of BLM-treated mice. However, ATX activity assays revealed that despite the elevated ATX levels in plasma, BrP administration resulted in reduced ATX activity, both in saline- and BLM-treated mice (FIG. 20C). Unfortunately we failed to determine ATX activity in BAL of mice most probably due to the extended dilution of the protein (lavage with 3 ml of saline).

Finally, since LPAR1 deficiency was found to reduce vascular leak in the course of BLM induced pulmonary fibrosis, we assessed total protein content in BAL fluids from vehicle- and BrP-treated mice. As can be seen in FIG. 20D, total protein content did not differ between the two experimental groups although values in BrP-treated group mice displayed great variation.

Similar results were also obtained with GWJ-A-23 a novel alpha-substituted phosphonate analogue of S32826 and potent specific inhibitor of ATX (Ferry, G., et al. S32826, a nanomolar inhibitor of autotaxin: discovery, synthesis and applications as a pharmacological tool. J Pharmacol Exp Ther 327, 809-819 (2008)), see FIG. 37

More importantly, we have developed monoclonal antibodies against ATX exhibiting good specificity, while able to inhibit ATX's enzymatic assay. These novel biological agents against ATX (a-ATX clones 1F8, 5H3, 2G5, and various combinations) will be administered in increasing concentrations (5, 10 and 20 mg/Kg) either prophylactically or therapeutically. Biological specific inactivation is expected to attenuate disease symptoms in accordance with genetic or small molecule inhibition of ATX.

To examine if pulmonary inflammation and fibrosis lead to altered phospholipid homeostasis various phospholipids in BALF were analyzed with LC/MS, showing significant differences (FIG. 21). Pending the validation in human samples the phospholipid profile could possibly predict disease classification and treatment responsiveness.

The following materials and methods were used in Example 12 and 13 as needed.

Animals.

All mice were bred at the animal facilities of the Alexander Fleming Biomedical Sciences Research Center, under specific pathogen-free conditions. Mice were housed at 20-22° C., 55±5% humidity. and a 12-h light-dark cycle; water and food was given ad libitum. CC10Cre^{+/-} mice were kept in a mixed CBA/C57.Bl6 background while Enpp2^{n/n} mice were bred on a C57.Bl6 background. Cohorts of CC10Cre^{+/-}/Enpp2^{n/n} (hereafter CC10Enpp2^{n/n}) and their Enpp2^{n/n} littermates were generated by crossing CC10Cre^{+/-}Enpp2^{n/n} mice with Enpp2^{n/n} mice.

Lung Cancer Animal Model.

Age and sex matched 6-8 week old mice were divided into an experimental group (CC10Enpp2^{n/n}: n=3, Enpp2^{n/n}: n=4) that received weekly i.p. injections of 1 g/Kg urethane for ten weeks and a control group (CC10Enpp2^{n/n}: n=3, Enpp2^{n/n}: n=3) that received saline i.p. injections instead. All mice were sacrificed at 24 weeks after urethane initiation for disease evaluation.

Histology and Immunocytochemistry.

Upon euthanasia, blood was drawn from the vena cava and was added to 50 mM EDTA for plasma preparations. Lungs were lavaged with an aliquot of 1 ml normal saline followed by 2 ml of saline (Bronchoalveolar Lavage Fluid, BALF). BALF was centrifuged and cell pellets was used for total cell counts with Trypan Blue while supernatants were snap frozen. Lungs were then perfused with 10 ml of PBS through the spontaneous beating right ventricle and instilled with 1 ml of 10% neutral buffered formalin. After dissection lungs fixed in 10% neutral buffered formalin. followed by 70% ethanol. Surface tumors were counted by three blinded readers under a dissecting microscope and averaged. Tissues were embedded in paraffin, and 5-μm-thick sections were cut at the median transverse level of the lungs. Sections were mounted on glass slides and stained with H&E. Neoplastic lesions were counted by three blinded readers and averaged.

Immunohistochemistry.

Immunostaining was performed with peroxidase labeling techniques. Tissue sections were deparaffinized, and endogenous peroxidase activity was blocked by incubation in 1% peroxide. The sections were preincubated with 2% FBS in PBS-Tween for 30 minutes, followed by incubation overnight with the primary antibody. Sections were then washed in PBS-T and incubated for 30 minutes with horseradish peroxidase (HRP)-conjugated anti-rabbit IgG (1:1000 dilution in PBS-T) or with. The sections were further washed with PBS-T. Finally. color was developed by immersing the sections in a solution of 0.05% 3,3′-diaminobenzidine (DAB; Sigma) and 0.01% hydrogen peroxide in PBS. The sections were counterstained with hematoxylin. For immunofluorescence, sections treated as above were incubated for 30 min with AlexaFluor-488 or -555 conjugated secondary antibodies. Finally, sections were further stained with DAPI for 5 min before mounting.

### Example 12

### Efficient Specific Recombination of Enpp2 Gene in the Clara Cell of the Lung

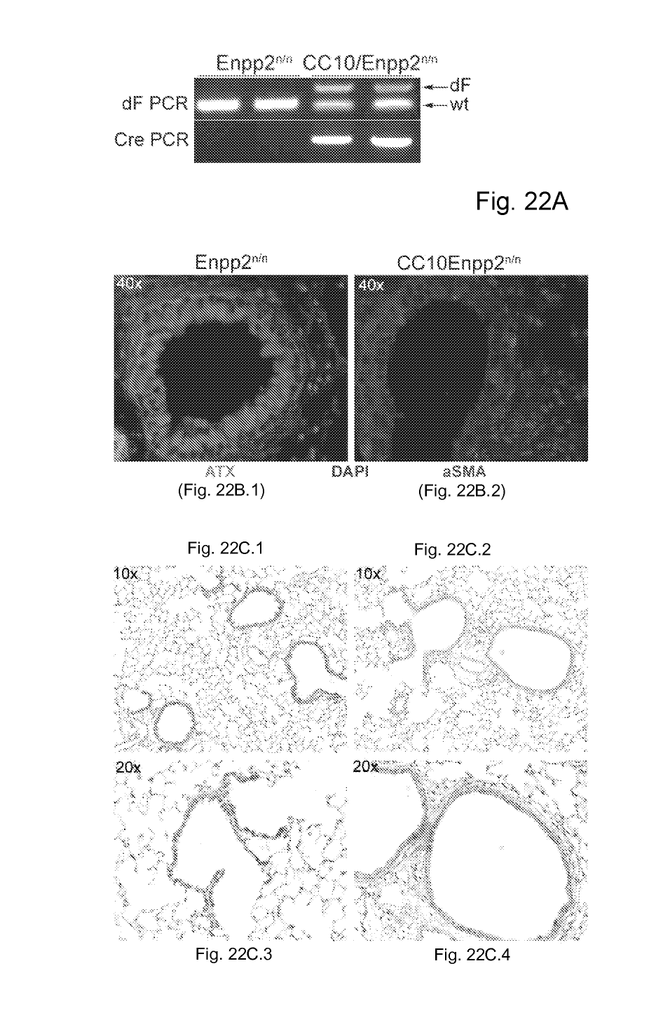

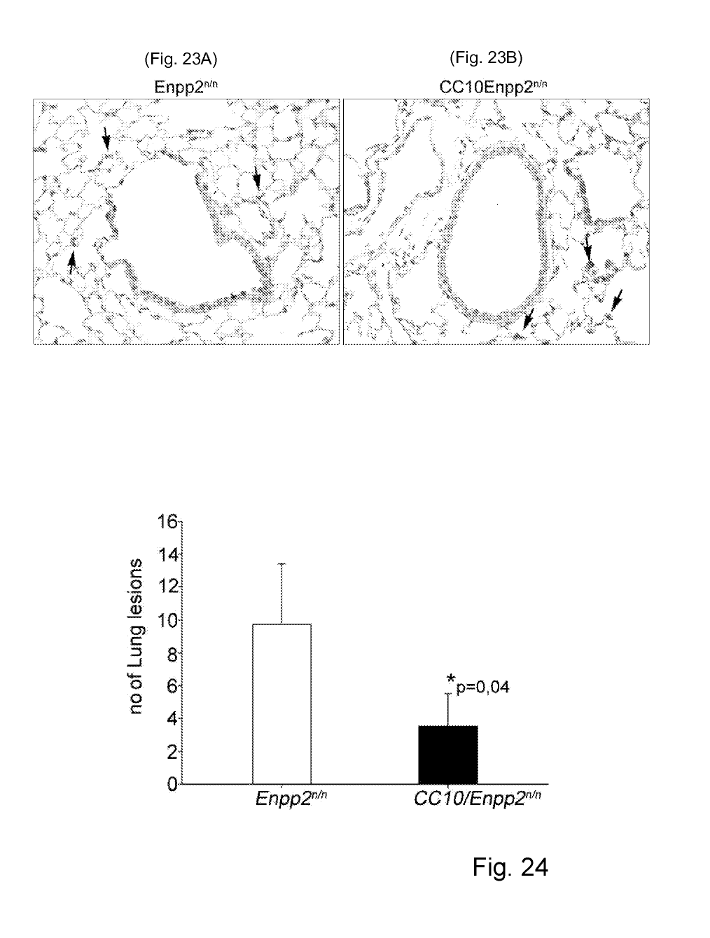

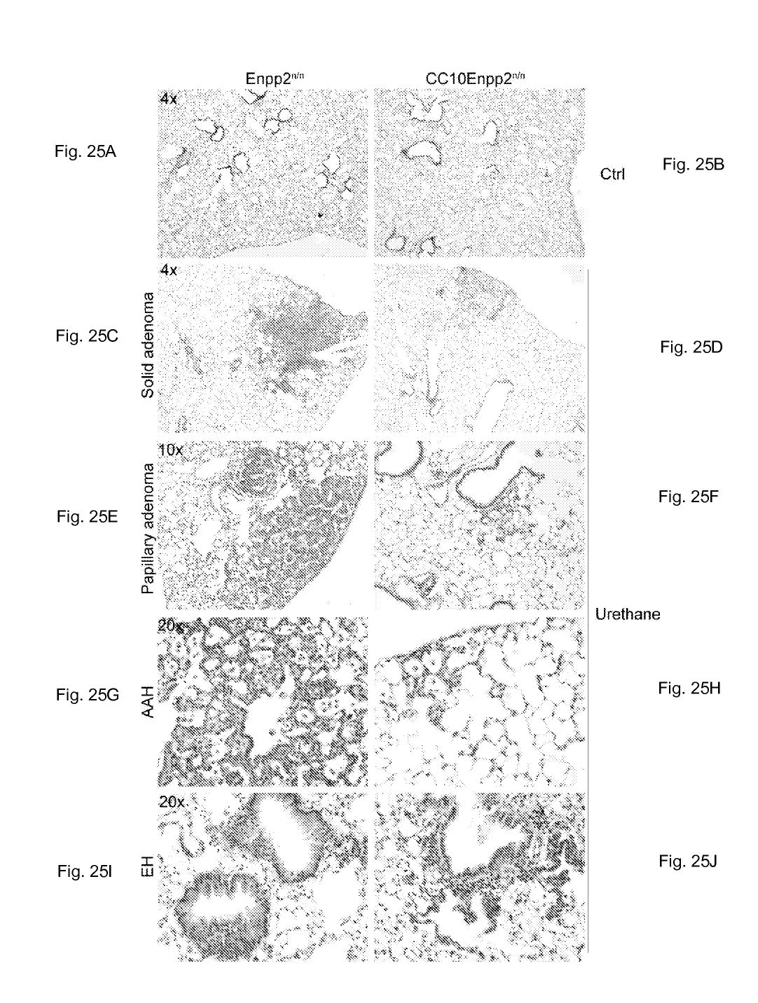

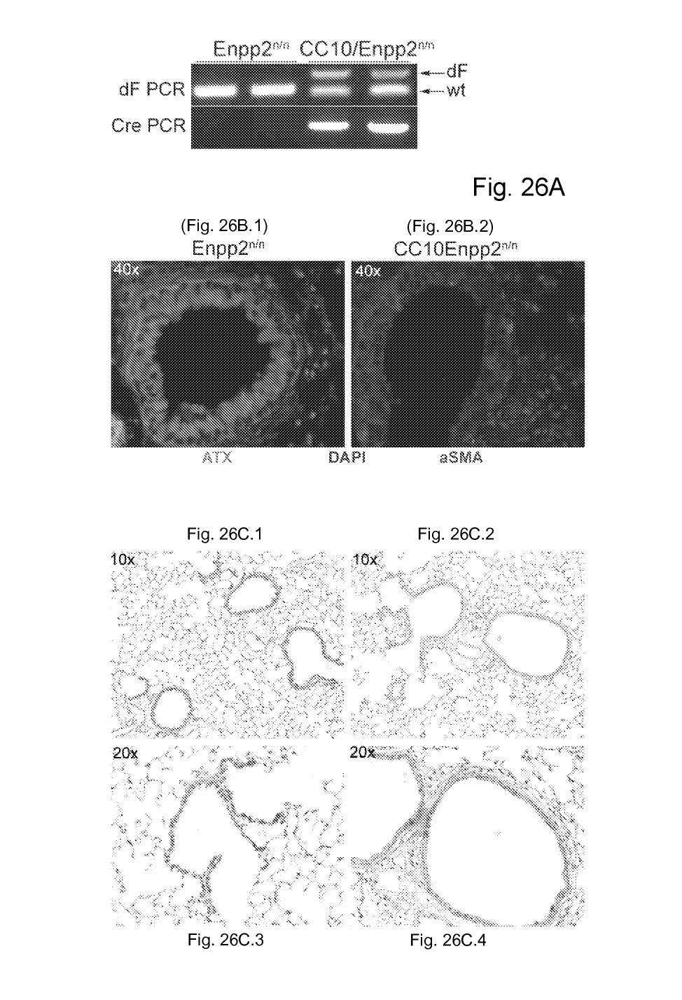

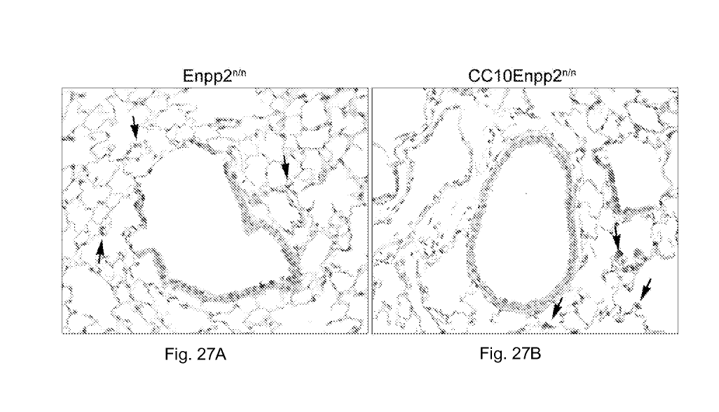

To explore a possible role of ATX and related LPA signalling in the pathogenesis of urethane-induced lung cancer, we conditionally ablated ATX expression in Clara cells of the lungs by mating the conditional Enpp2^{n/n} mice with CC10Cre^{+/-} mice. Efficient recombination of floxed alleles was evaluated by PCR detecting the defloxed band in DNA samples derived from lung tissue sections and immunocytochemistry. As can be seen in FIG. 22A, the defloxed band (dF) is only present in the lungs of CC10Enpp2^{n/n} mice that express the Cre enzyme.

Moreover immunocytochemistry experiments revealed the absence of ATX staining in the majority of epithelial cells of both the major bronchi and the respiratory bronchioles, as can been seen in FIGS. 1B,C. ATX was also found to be expressed by alveolar type II epithelial cells (arrows in FIG. 23) but expression levels were lower than that of Clara cells. ATX deletion in Clara cells did not interfere with type II epithelial cell ATX expression (FIG. 23).

### Example 13

### Specific Inactivation of ATX from Clara Cells Results in Reduced Tumor Formation in Urethane-Induced Lung Cancer

In order to investigate the effect of Enpp2 gene inactivation specifically in the Clara cells of the bronchi during lung cancer development we injected mice intraperitoneally with urethane for 10 consecutive weeks and sacrificed mice 14 weeks later for lung tumor enumeration. As can be seen in FIG. 24. CC10Enpp2^{n/n} mice had reduced number of tumors on the surface of their lungs (9.75±3.6 versus 3.5±1.9 for Enpp2^{n/n} and CC10Enpp2^{n/n} respectively). Histopathologic analysis of lung sections revealed that urethane, as expected, caused a range of different neoplastic lesions: atypical adenomatous hyperplasia (AAH), bronchiolar hyperplasia (EH), papillary and solid adenoma (AD), and adenocarcinoma (AC). Conditional ATX deletion from the bronchiolar epithelium had as a result the incomplete formation of each one of the above neoplastic lesions. Of note, this mouse strain exhibited foci of AAH that involved only few neighbouring alveoli, in contrast to Enpp2^{n/n} mice that presented more organized AAH lesions that occupied larger areas of the alveolar epithelium (FIG. 25). Moreover, Enpp2^{n/n} mice had increased numbers of adenomas that were characterized mainly of papillary rather than solid structure whereas AAH lesions of CC10Enpp2^{n/n} seemed that never progressed to adenomas, contributing to the reduced formation of AD in their lungs. Occasionally some bronchi with EH were present in the lungs of Enpp2^{n/n} mice (FIG. 25, EH) with much lower incidence in the lungs of CC10Enpp2^{n/n} mice. Inflammation was not a prominent histopathologic feature in either mouse strain although the presence of macrophages (giant cells) was more frequent in the lungs of CC10Enpp2^{n/n} mice in close proximity to AAH lesions.

The following materials and methods were used as needed for Examples 14-19.

Animals.

All mice were bred at the animal facilities of the Alexander Fleming Biomedical Sciences Research Center, under specific pathogen-free conditions.

Mice were housed at 20-22° C., 55±5% humidity, and a 12-h light-dark cycle; water and food was given ad libitum. CC10Cre^{+/-} mice were kept in a mixed CBA/C57.Bl6 background while Enpp2^{n/n} mice were bred on a C57.Bl6 background. Cohorts of CC10Cre^{+/-}/Enpp2^{n/n} (hereafter CC10Enpp2^{n/n}) and their Enpp2^{n/n} littermates were generated by crossing CC10Cre^{+/-}Enpp2^{n/n} mice with Enpp2^{n/n} mice. Enpp2-Tg^{+/-} transgenic mice have been described previously and were kept in an mixed FVB-C57BL/6 genetic background. Finally Enpp2^{+/-} mice were kept in a C57/BL6 genetic background.

LPS Animal Model.

6-8 week old littermate mice were divided into an experimental group that received aerosolised LPS (from Pseudomonas aeruginosa, 10 mg dissolved in 3 ml Normal Saline) and a control group that received Normal Saline instead. For inhibition studies WT mice received an intratracheal injection of the specific ATX/LPAR inhibitor HLZ at two doses (100 μg/Kg and 200 μg/Kg) immediately prior to LPS administration. The LPS solution was administered by a custom-made nebuliser flowing at 4 l/min oxygen for 20-25 min into an airtight chamber containing 5-7 mice. All analyses were performed 24 h later.

Histology and Immunocytochemistry.

Upon euthanasia, blood was drawn from the vena cava and was added to 50 mM EDTA for plasma preparations. Lungs were lavaged with an aliquot of 1 ml normal saline followed by 2 ml of saline (Bronchoalveolar Lavage Fluid. BALF). BALF aliquots were centrifuged; the cell pellets were combined and used for total cell counts with Trypan Blue while supernatants were snap-frozen. Lungs were then perfused with 10 ml of PBS through the spontaneous beating right ventricle and the left lung was snap-frozen for future analysis while the right lung was fixed in 10% neutral buffered formalin. Tissues were embedded in paraffin, and 5-μm-thick sections were cut at the median transverse level of the lungs. Sections were mounted on glass slides and stained with H&E.

Immunohistochemistry.

Immunostaining was performed with peroxidase labeling techniques. Tissue sections were deparaffinized. and endogenous peroxidase activity was blocked by incubation in 1% peroxide. The sections were preincubated with 2% FBS in PBS-Tween for 30 minutes, followed by incubation overnight with the primary antibody. Sections were then washed in PBS-T and incubated for 30 minutes with horseradish peroxidase (HRP)-conjugated anti-rabbit IgG (1:1000 dilution in PBS-T) or with. The sections were further washed with PBS-T. Finally, color was developed by immersing the sections in a solution of 0.05% 3,3′-diaminobenzidine (DAB: Sigma) and 0.01% hydrogen peroxide in PBS. The sections were counterstained with hematoxylin. For immunofluorescence, sections treated as above were incubated for 30 min with AlexaFluor-488 or -555 conjugated secondary antibodies. Finally, sections were further stained with DAPI for 5 min before mounting.

ATX Activity Assay.

Autotaxin activity in BALF of mice was determined using the cleavage of the ATX-specific fluorogenic substrate FS-3 (Echelon Biosciences, UT, USA). Assays were conducted in a final volume of 100 μl comprising 20 μl 5× assay buffer (700 mM NaCl, 25 mM KCl, 5 mM MgCl₂ and 250 mM Tris, pH 8.0), 20 μl of FS-3 (final concentration 2.5 μM) and 60 μl of BALF (diluted 1:2 in 1×PBS). Reactions were incubated at 37° C. in a Tecan Infinite 200 microplate reader (Tecan Trading AG, Switzerland) set to make fluorescence measurements every 5 min for 1 hour. Autotaxin activity was quantitated by measuring the rate of increase in fluorescence at 528 nm with excitation at 485 nm.

Total Protein Content Determination.

Total protein concentration in BALFs was measured using the Bradford protein assay (Biorad, Hercules, Calif.) according to manufacturer's recommendations. OD readings of samples were converted to μg/ml using values obtained from a standard curve generated with serial dilutions of bovine serum albumin (50-500 μg/ml).

### ATX ELISA

To detect ATX in biological fluids, a direct ELISA assay was developed using the commercially available anti-phospholipase D polyclonal antibody (Cat. No. 10005375, Cayman chemical, USA). The bottom of a NUNC-IMMUNO 96 MicroWell Elisa Plate was coated overnight with 100 μl of 1:200 diluted serum in 0.05 M carbonate/bicarbonate coating buffer, pH 9.5 (Cat. No. S7795/7277, Sigma, USA), washed three times with 0.05% Tween-20 (Cat. No. P1379, Sigma, USA) in TBS and blocked with 0.1% Bovine Serum Albumin (Cat. No. A7888, Sigma, USA) in 0.05% TBST for 1 hour at RT. For each ELISA assay, 100 μl of two-fold serial dilutions of recombinant ATX protein ranging from 4 to 0.5 μg/ml ware plated as standard curve and treated as all samples. The plate was then incubated with 0.5 μg/ml detection antibody for 1 hour and washed three times with 0.05% TBST. Autotaxin antigen was then detected with an anti-rabbit HRP-labeled secondary antibody (Cat. No. 4010-05, SouthernBiotech. USA) that was developed with TMB substrate (Cat. No. A7888, Sigma, USA). Readings were obtained at 450 nm. All samples and standards were assayed in triplicates.

### Example 14

### Efficient Specific Recombination of Enpp2 gene in the Clara Cells of the Lung

To explore a possible role of ATX and related LPA signalling in the pathogenesis of urethane-induced lung cancer, we conditionally ablated ATX expression in Clara cells of the lungs by mating the conditional Enpp2^{n/n} mice with CC10Cre^{+/-} mice. Efficient recombination of floxed alleles was evaluated by PCR detecting the defloxed band in DNA samples derived from lung tissue sections and immunocytochemistry. As can be seen in FIG. 26A, the defloxed band (dF) is only present in the lungs of CC10Enpp2^{n/n} mice that express the Cre enzyme.

Moreover immunocytochemistry experiments revealed the absence of ATX staining in the majority of epithelial cells of both the major bronchi and the respiratory bronchioles, as can been seen in FIGS. 26B,C. ATX was also found to be expressed by alveolar type II epithelial cells (arrows in FIG. 27) but expression levels were lower than that of Clara cells. ATX deletion in Clara cells did not interfere with type II epithelial cell ATX expression (FIG. 27).

### Example 15

### Specific Inactivation of ATX from Clara Cells Results in Increased Pulmonary Inflammation after Aeroeolised LPS Administration

In order to investigate the effect of the Enpp2 gene inactivation specifically in the Clara cells of the bronchi after LPS challenge, we administrated aerosolised LPS and sacrificed mice 24 hours later for lung pathology evaluation. As can be seen in FIG. 28, both histopathologic examination and total cellularity counts revealed that CC10Enpp2 mice developed an increased neutrophilic lung inflammation (FIGS. 28A and B. respectively). LPS administration in WT mice resulted in an increase in ATX's enzymatic activity when compared to saline-treated control mice, an effect that was attenuated in CC10Enpp2 mice after LPS challenge (FIG. 28C). suggesting that CC10⁺ bronchiolar epithelial cells may represent an important cellular source for the production and secretion of ATX. Furthermore, the increased neutrophil infiltration into the lungs of CC10Enpp2 mice after LPS exposure was correlated with significant increases in BAL inflammatory indices such as BAL protein content (FIG. 28D). indicating that the lower levels of ATX's activity could account for the observed alveolar-capillary barrier disruption.

### Example 16

### Pharmacologic Inhibition of ATX Exacerbates LPS-Induced Pulmonary Inflammation

BrP-LPA is a dual specific inhibitor of both ATX and LPA receptors [1]. In order to test whether the pharmacologic inhibition of the ATX/LPA axis would affect lung pathology after LPS administration. BrP-LPA was delivered intratracheally in WT mice prior to LPS exposure. BrP-LPA treated mice exhibited significant increases in LPS-induced lung inflammation and alveolar edema at 24 hr, as determined by histopathologic examination and BALF's total cell counts (FIGS. 29A and B). Contrary to what was expected, activity assays in BAL fluids failed to detect lower levels of enzymatic activity (FIG. 29C). A possible explanation for this paradox would be that either the selected dosing of the inhibitor did not suffice to completely inhibit ATX's activity, at least at the 24 hr time point or this discrepancy could reflect the existence of positive feedback regulatory mechanisms that could account for the increased activity levels. Nevertheless, BrP-LPA treatment had a dramatic effect in the alveolar-capillary barrier function since BAL protein content was significantly elevated when compared to vehicle-treated mice (FIG. 29D). Of note, BrP-LPA also increased total protein concentration in BAL fluids derived from saline treated-mice whose lungs displayed enlarged, dilated airspaces as can be seen in the respective H/E stained lung sections (FIG. 29A). These results may imply a broader role for the ATX/LPA system in regulating normal and pathological lung function.

### Example 17

### Genetic Overexpression of Enpp2/ATX Attenuate LPS-Induced Lung Pathology

Since genetic or pharmacologic inhibition of ATX deteriorates LPS-induced pulmonary inflammation, we then assessed whether overexpression of ATX would have a beneficial role in the development of LPS-induced lung injury. To this end we used the Enpp2-Tg^{+/-} transgenic mice that have been previously described [2]. In these mice the human ATX cDNA was placed under the control of the promoter of the human a1 antitrypsin gene which is predominantly expressed by liver cells. These mice are reported to contain increased plasma ATX levels (range 1.3-3.6 fold) and a corresponding increase in plasma ATX activity. Moreover and since the a1 antitrypsin gene is also expressed by lung epithelial cells, these transgenic mice were reported to overexpress ATX by lung tissue as well. So in order to investigate the effects of LPS administration in the setting of increased circulating ATX levels we exposed Enpp2-Tg^{+/-} mice and their littermates in aerosolized LPS and evaluated lung pathology 24 hr later. As expected Enpp2-Tg^{+/-} mice displayed significant reduced lung inflammation as judged by histopathologic examination and total cell counts (FIGS. 30A and B, respectively). Both basal and LPS-induced ATX activity was found to be slightly elevated but not statistically different from control WT mice (FIG. 30C). Although not statistically different between WT and Enpp2-Tg^{+/-} mice, total BAL protein content was reduced in the latter after LPS exposure (FIG. 30D). Finally MPO assays, a sensitive marker of neutrophil accumulation, confirmed the attenuated infiltration of neutrophils in the lungs of Enpp2-Tg^{+/-} mice (FIG. 30E). In conclusion, using a mouse strain that overexpresses ATX systemically by the liver, we were able to demonstrate important decreases in LPS-induced lung inflammation despite the fact that this phenomenon could not be directly correlated with statistical significant differences in either BAL ATX activity or total protein measurements. Direct instillation of recombinant ATX protein into the lungs of WT mice or the use of lung overexpressing transgenic mouse strains would be a more preferable choice for studying the impact of increased ATX levels in the mechanisms of LPS-induced lung inflammation.

### Example 18

### Enpp2 Heterozygous Mice Display Enhanced Pulmonary Inflammation after LPS Exposure

Finally, we used the Enpp2^{+/-} mice which have been reported to contain half levels of ATX in their plasma and consequently half ATX activity and half LPA levels [3]. LPS treatment in these mice resulted, as expected, in elevated numbers of infiltrating neutrophils (FIGS. 31A and B) when compared to their WT littermates. Although BAL ATX activity in naïve Enpp2^{+/-} mice was found to be significantly diminished compared to naïve WT mice. LPS induced ATX activity to comparable levels in both mouse strains (FIG. 31B) suggesting that even one copy of the gene suffices for proper protein production. BAL total protein content was reduced in the case of Enpp2^{+/-} mice albeit not significantly (FIG. 31C). These data may point to an early role of ATX in the course of pulmonary inflammation, acting soon after LPS administration and before the production of signals that induce its expression.

### Example 19

### Altered Phospholipid Homeostatis Upon Acute Lung Injury

As modulation of ATX levels had a profound effect in the pathogenesis of LPS-induced acute lung injury we next assessed if acute lung injury has also an effect in the levels of various phospholipids in the BAL fluids of challenged mice. As evident from FIG. 32, LPA administration resulted in the modulation of various phospholipids. Therefore measuring the relative phospholipid levels, as well as the levels of the corresponding enzymes that metabolize the various phospholipids, might have a prognostic value in the severity of the lung injury. Towards this end, we are currently analyzing a cohort of septic and septic shock intensive care patients.

Based on the above results and in order to explore for a therapeutic role of ATX in LPS-acute lung injury, we intratracheally instilled rATX (750 ng) in WT (C57/Bl6) mice prior to LPS exposure. As can be seen in FIG. 33, rATX supplementation had as a result the diminished infiltration of inflammatory cells in the lungs of mice an observation that guarantees further exploration of the therapeutic use of ATX in experimental acute lung injury.

More importantly, since it has recently been shown that exogenous administration of either LPC or LPA, the enzymatic substrate and product respectively of ATX, results in significant reduced lethality in the respective animal models of sepsis, we sought to determine whether circulating levels of ATX are affected during the development of sepsis in humans. To this end blood was isolated from patients at baseline and after the development of sepsis and plasma preparations were made. ATX quantification in these samples revealed that circulating ATX protein levels are reduced upon sepsis development (FIG. 34). Because ATX is the major enzyme for the conversion of LPC to LPA in bodily fluids these changes in plasma ATX levels could possibly reflect alterations in the relative levels of these major phospholipds with as yet unknown consequences in sepsis development.

### Administration

If desired, pharmaceutically acceptable binding agents and adjuvants may comprise part of the formulated drug for use with the invention. Capsules, tablets and pills etc. may contain for example the following compounds: microcrystalline cellulose, gum or gelatin as binders; starch or lactose as excipients; stearates as lubricants; various sweetening or flavouring agents. For capsules the dosage unit may contain a liquid carrier like fatty oils. Likewise coatings of sugar or enteric agents may be part of the dosage unit. The oligonucleotide formulations may also be emulsions of the active pharmaceutical ingredients and a lipid forming a micellular emulsion. A compound described herein, for example, may be mixed with any material that does not impair the desired action, or with material that supplement the desired action. These could include other drugs including other nucleotide compounds. For parenteral, subcutaneous, intradermal or topical administration the formulation may include a sterile diluent, buffers, regulators of tonicity and antibacterials. The active compound may be prepared with carriers that protect against degradation or immediate elimination from the body, including implants or microcapsules with controlled release properties. For intravenous administration the preferred carriers are physiological saline or phosphate buffered saline.

By way of illustration, an ATX inhibitor can be included in a unit formulation such as in a pharmaceutically acceptable carrier or diluent in an amount sufficient to deliver to a patient a therapeutically effective amount without causing serious side effects in the treated patient.

A compound for use with the invention may be administered in a number of ways depending upon whether local or systemic treatment is desired and upon the area to be treated. Administration may be (a) oral (b) pulmonary, e.g., by inhalation or insufflation of powders or aerosols, including by nebulizer; intratracheal, intranasal, (c) topical including epidermal, transdermal, ophthalmic and to mucous membranes including vaginal and rectal delivery; or (d) parenteral including intravenous, intraarterial, subcutaneous, intraperitoneal or intramuscular injection or infusion; or intracranial, e.g., intrathecal or intraventricular, administration. In one embodiment the pharmaceutical composition is administered IV, IP, orally, topically or as a bolus injection or administered directly in to the target organ. Pharmaceutical compositions and formulations for topical administration may include transdermal patches, ointments, lotions, creams, gels, drops, sprays, suppositories, liquids and powders. Conventional pharmaceutical carriers, aqueous, powder or oily bases, thickeners and the like may be necessary or desirable. Coated condoms, gloves and the like may also be useful. Preferred topical formulations include those in which the compounds of the invention are in admixture with a topical delivery agent such as lipids, liposomes, fatty acids, fatty acid esters, steroids, chelating agents and surfactants. Compositions and formulations for oral administration include but is not restricted to powders or granules, microparticulates, nanoparticulates, suspensions or solutions in water or non-aqueous media, capsules, gel capsules, sachets, tablets or minitablets. Compositions and formulations for parenteral, intrathecal or intraventricular administration may include sterile aqueous solutions which may also contain buffers, diluents and other suitable additives such as, but not limited to, penetration enhancers, carrier compounds and other pharmaceutically acceptable carriers or excipients. Particular concentrations of the compounds used herein will be guided by recognized parameters such as the sex and general health of the particular patient. For most applications administration of between from about 1 pg to 200 mg/kg or more will be useful to achieve a desired effect.

The disclosures of the following references are incorporated by reference.
1. Stracke, M. L., Clair, T. & Liotta, L. A. Autotaxin, tumor motility-stimulating exophosphodiesterase. Advances in enzyme regulation 37, 135-144 (1997).
2. Stracke,. M. L., et al. Identification, purification, and partial sequence analysis of autotaxin, a novel motility-stimulating protein. J Biol Chem 267, 2524-2529 (1992).
3. Giganti, A. et al. Murine and Human Autotaxin {alpha}. {beta}, and {gamma}Isoforms: Gene organization, tissue distribution and biochemical characterization. J Biol Chem 283, 7776-7789 (2008).
4. van Meeteren, L. A. & Moolenaar, W. H. Regulation and biological activities of the autotaxin-LPA axis. Prog Lipid Res 46, 145-160 (2007).
5. Jansen, S., et al. Proteolytic maturation and activation of autotaxin (NPP2), a secreted metastasis-enhancing lysophospholipase D. J Cell Sci 118, 3081-3089 (2005).
6. Stefan, C., Jansen, S. & Bollen, M. NPP-type ectophosphodiesterases: unity in diversity. Trends Biochem Sci 30, 542-550 (2005).
7. Umezu-Goto, M., et al. Autotaxin has lysophospholipase D activity leading to tumor cell growth and motility by lysophosphatidic acid production. J Cell Biol 158, 227-233 (2002).
8. Croset, M. Brossard, N., Polette, A. & Lagarde, M. Characterization of plasma unsaturated lysophosphatidylcholines in human and rat. Biochem J 345 Pt 1, 61-67 (2000).
9. Moolenaar, W. H., van Meeteren, L. A. & Giepmans, B. N. The ins and outs of lysophosphatidic acid signaling. Bioessays 26, 870-881 (2004).
10. Goetzl, E. J., et al. Gelsolin binding and cellular presentation of lysophosphatidic acid. J Biol Chem 275, 14573-14578 (2000).
11. Tigyi, G. & Miledi, R. Lysophosphatidates bound to serum albumin activate membrane currents in Xenopus oocytes and neurite retraction in PC12 pheochromocytoma cells. J Biol Chem 267, 21360-21367 (1992).
12. Contos, J. J. Ishii, I. & Chun. J. Lysophosphatidic acid receptors. Mol Pharmacol 68, 1188-1196 (2000).
13. Aidinis, V., et al. Cytoskeletal rearrangements in synovial fibroblasts as a novel pathophysiological determinant of modeled rheumatoid arthritis. PLoS genetics 1, e48 (2005).
14. Kehlen, A., et al. IL-1 beta- and IL-4-induced down-regulation of autotaxin mRNA and PC-1 in fibroblast-like synoviocytes of patients with rheumatoid arthritis (RA). Clinical and experimental immunology 123, 147-154 (2001).
15. Santos, A. N., et al. Treatment of fibroblast-like synoviocytes with IFN-gamma results in the down-regulation of autotaxin mRNA. Biochem Biophys Res Commun 229, 419-424 (1996).
16. Zhao, C., et al. Regulation of lysophosphatidic acid receptor expression and function in human synoviocytes: implications for rheumatoid arthritis? Molecular pharmacology 73, 587-600 (2008).
17. Fotopoulou, S., et al. ATX expression and LPA signalling are vital for the development of the nervous system. Dev Biol under revision (2009).
18. Armaka, M., et al. Mesenchymal cell targeting by TNF as a common pathogenic principle in chronic inflammatory joint and intestinal diseases. The Journal of experimental medicine 205, 331-337 (2008).
19. Keffer, J., et al. Transgenic mice expressing human tumour necrosis factor: a predictive genetic model of arthritis. The EMBO journal 10, 4025-4031 (1991).
20. Li, P. & Schwarz, E. M. The TNF-alpha transgenic mouse model of inflammatory arthritis. Springer Semin Immunopathol 25, 19-33 (2003).
21. Kontoyiannis, D. Pasparakis, M. Pizarro, T. T., Cominelli, F. & Kollias, G. Impaired on/off regulation of TNF biosynthesis in mice lacking TNF AU-rich elements: implications for joint and gut-associated immunopathologies. Immunity 10, 387-398 (1999).
22. Campbell, I. K., Hamilton, J. A. & Wicks, I. P. Collagen-induced arthritis in C57BL/6 (H-2b) mice: new insights into an important disease model of rheumatoid arthritis. European journal of immunology 80, 1568-1575 (2000).
23. Pamuklar, Z. et al. Autotaxin/lysopholipase D and Lysophosphatidic Acid Regulate Murine Hemostasis and Thrombosis. The Journal of biological chemistry (2009).
24. Xu, X. & Prestwich, G. D. Inhibition of tumor growth and angiogenesis by a lysophosphatidic acid antagonist in an engineered three-dimensional lung cancer xenograft model. Cancer 116, 1739-1750.
25. Xu, X., Yang, G., Zhang, H. & Prestwich, G. D. Evaluating dual activity LPA receptor pan-antagonist/autotaxin inhibitors as anti-cancer agents in vivo using engineered human tumors. Prostaglandins Other Lipid Mediat (2009).
26. Aidinis, V., et al. Functional analysis of an arthritogenic synovial fibroblast. Arthritis research & therapy 5. R140-157 (2003).
27. Fuchs, B. & Schiller, J. Lysophospholipids: their generation, physiological role and detection. Are they important disease markers? Mini Rev Med Chem 9, 368-378 (2009).
28. Fuss, B., Baba, H., Phan, T., Tuohy, V. K. & Macklin, W. B. Phosphodiesterase I, a novel adhesion molecule and/or cytokine involved in oligodendrocyte function. J Neurosci 17, 9095-9103 (1997).
29. Kawagoe, H., et al. Molecular cloning and chromosomal assignment of the human brain-type phosphodiesterase I/nucleotide pyrophosphatase gene (PDNP2). Genomics 80, 380-384 (1995).
30. Lee, H. Y., et al. Stimulation of tumor cell motility linked to phosphodiesterase catalytic site of autotaxin. J Biol Chem 271, 24408-24412 (1996).
31. Narita, M., Goji, J., Nakamura, H. & Sano, K. Molecular cloning, expression, and localization of a brain-specific phosphodiesterase I/nucleotide pyrophosphatase (PD-1 alpha) from rat brain. J Biol Chem 269, 28235-28242 (1994).
32. Bachner, D., Ahrens, M., Betat, N., Schrqpder, D. & Gross, G. Developmental expression analysis of murine autotaxin (ATX). Mechanisms of Development 84, 121-125 (1999).
33. Awatramani, R., Soriano, P., Rodriguez, C., Mai. J. J. & Dymecki,. S. M. Cryptic boundaries in roof plate and choroid plexus identified by intersectional gene activation. Nat Genet 835, 70-75 (2003).
34. Li, Y., Chen, J. & Chopp, M. Cell proliferation and differentiation from ependymal, subependymal and choroid plexus cells in response to stroke in rats. J Neurol Sci 198, 137-146 (2002).
35. Dugas, J. C., Tai, Y. C., Speed, T. P., Ngai, J. & Barres, B. A. Functional genomic analysis of oligodendrocyte differentiation. J Neurosci 26, 10967-10983 (2006).
36. Fox, M. A., Alexander, J. K., Afshari, F. S., Colello, R. J. & Fuss, B. Phosphodiesterase-I[alpha]/autotaxin controls cytoskeletal organization and FAK phosphorylation during myelination. Molecular and Cellular Neuroscience 27, 140-150 (2004).
37. Hoelzinger, D.B., et al. Gene expression profile of glioblastoma multiforme invasive phenotype points to new therapeutic targets. Neoplasia 7, 7-16 (2005).
38. Sato, K., et al. Identification of autotaxin as a neurite retraction-inducing factor of PC12 cells in cerebrospinal fluid and its possible sources. J Neurochem 92, 904-914 (2005).
39. Savaskan, N. E., et al. Autotaxin (NPP-2) in the brain: cell type-specific expression and regulation during development and after neurotrauma. Cell Mol Life Sci 64, 230-243 (2007).
40. Ramakers. G. J. & Moolenaar, W. H. Regulation of astrocyte morphology by RhoA and lysophosphatidic acid. Exp Cell Res 245, 252-262 (1998).
41. van Meeteren, L. A., et al. Inhibition of Autotaxin by Lysophosphatidic Acid and Sphingosine 1-Phosphate. J Biol Chem 280, 21155-21161 (2005).
42. Nam, S. W., et al. Autotaxin (ATX). a potent tumor motogen, augments invasive and metastatic potential of ras-transformed cells. Oncogene 19, 241-247 (2000).
43. Kawagoe, H., Stracke, M. L., Nakamura, H. & Sano, K. Expression and Transcriptional Regulation of the PD-I{alpha}/Autotaxin Gene in Neuroblastoma. Cancer Res 57, 2516-2521 (1997).
44. DufnerBeattie, J., Lemons, R. S. & Thorburn, A. Retinoic acid-induced expression of autotaxin in N-myc-amplified neuroblastoma cells. Mol Carcinog 80, 181-189 (2001).
45. Umemura, K., et al. Autotaxin expression is enhanced in frontal cortex of Alzheimer-type dementia patients. Neuroscience Letters 400, 97-100 (2006).
46. Hammack, B. N., et al. Proteomic analysis of multiple sclerosis cerebrospinal fluid. Mult Scler 10, 245-260 (2004).
47. Dennis, J., Nogaroli, L. & Fuss, B. Phosphodiesterase-Ialpha/autotaxin (PD-Ialpha/ATX): a multifunctional protein involved in central nervous system development and disease. J Neurosci Res 82, 737-742 (2005).
48. Fukushima, N., Weiner, J. A. & Chun, J. Lysophosphatidic acid (LPA) is a novel extracellular regulator of cortical neuroblast morphology. Dev Biol 228, 6-18 (2000).
49. Brauer, A. U., et al. A new phospholipid phosphatase. PRG-1, is involved in axon growth and regenerative sprouting. Nat Neurosci 6, 572-578 (2003).
50. Holtsberg, F. W., et al. Lysophosphatidic acid and apoptosis of nerve growth factor-differentiated PC12 cells. J Neurosci Res 58, 685-696 (1998).
51. Kingsbury, M. A., Rehen, S. K., Contos, J. J., Higgins, C. M. & Chun, J. Non-proliferative effects of lysophosphatidic acid enhance cortical growth and folding. Nat Neurosci 6, 1292-1299 (2003).
52. Dawson, J., Hotchin, N. Lax, S. & Rumsby, M. Lysophosphatidic acid induces process retraction in CG-4 line oligodendrocytes and oligodendrocyte precursor cells but not in differentiated oligodendrocytes. J Neurochem 87, 947-957 (2003).
53. Schilling, T. Stock, C., Schwab, A. & Eder, C. Functional importance of Ca2+-activated K+ channels for lysophosphatidic acid-induced microglial migration. Eur J Neurosci 19, 1469-1474 (2004).
54. Steiner, M. R. Urso, J. R., Klein, J. & Steiner, S. M. Multiple astrocyte responses to lysophosphatidic acids. Biochimica et biophysica acta 1582, 154-160 (2002).
55. Kinouchi, H. Imaizumi, S., Yoshimoto, T., Yamamoto, H. & Motomiya, M. Changes of polyphosphoinositides, lysophospholipid, and free fatty acids in transient cerebral ischemia of rat brain. Mol Chem Neuropathol 12, 215-228 (1990).
56. Tigyi, G., et al. Lysophosphatidic acid alters cerebrovascular reactivity in piglets. Am J Physiol 268, H2048-2055 (1995).
57. Sayas, C. L., Moreno-Flores, M. T., Avila, J. & Wandosell, F. The neurite retraction induced by lysophosphatidic acid increases Alzheimer's disease-like Tau phosphorylation. J Biol Chem 274, 37046-37052 (1999).
58. Inoue, M. Ma, L., Aoki, J. Chun, J. & Ueda, H. Autotaxin, a synthetic enzyme of lysophosphatidic acid (LPA), mediates the induction of nerve-injured neuropathic pain. Molecular Pain 4, 6 (2008).
59. Contos. J. J. Fukushima, N. Weiner, J. A. Kaushal. D. & Chun, J. Requirement for the IpA1 lysophosphatidic acid receptor gene in normal suckling behavior. Proc Natl Acad Sci USA 97, 13384-13389 (2000).
60. Contos, J. J., et al. Characterization of lpa(2) (Edg4) and lpa(1)/lpa(2) (Edg2/Edg4) lysophosphatidic acid receptor knockout mice: signaling deficits without obvious phenotypic abnormality attributable to lpa(2). Mol Cell Biol 22, 6921-6929 (2002).
61. Jaillard, C., et al. Edg8/S1P5: an oligodendroglial receptor with dual function on process retraction and cell survival. J Neurosci 25, 1459-1469 (2005).
62. Saba, J. D. Lysophospholipids in development: Miles apart and edging in. Journal of cellular biochemistry 92, 967-992 (2004).
63. Weiner, J. A. & Chun. J. Schwann cell survival mediated by the signaling phospholipid lysophosphatidic acid. Proc Natl Acad Sci USA 96, 5233-5238 (1999).
64. Hovelmeyer, N., et al. Apoptosis of oligodendrocytes via Fas and TNF-R1 is a key event in the induction of experimental autoimmune encephalomyelitis. J Immunol 175, 5875-5884 (2005).
65. Fotopoulou, S., et al. ATX expression and LPA signalling are vital for the development of the nervous system. Dev Biol 339, 451-464 (2010).
66. Tager, A. M., et al. The lysophosphatidic acid receptor LPA1 links pulmonary fibrosis to lung injury by mediating fibroblast recruitment and vascular leak. Nat Med 14, 45-54 (2008).
67. Aoki, J. Mechanisms of lysophosphatidic acid production. Seminars in Cell & Developmental Biology 15, 477-489 (2004).
68. Xu, X. Yang, G. Zhang, H. & Prestwich, G. D. Evaluating dual activity LPA receptor pan-antagonist/autotaxin inhibitors as anti-cancer agents in vivo using engineered human tumors. Prostaglandins & Other Lipid Mediators 89, 140-146 (2009).
69. Prestwich, G. D., et al. Phosphatase-resistant analogues of lysophosphatidic acid: Agonists promote healing, antagonists and autotaxin inhibitors treat cancer. Biochimica et biophysica acta 8, 8 (2008).
70. Demedts, M. & Costabel, U. ATS/ERS international multidisciplinary consensus classification of the idiopathic interstitial pneumonias. Eur Respir J 19, 794-796 (2002).
71. Euer, N., et al. Identification of genes associated with metastasis of mammary carcinoma in metastatic versus non-metastatic cell lines. Anticancer Res 22, 733-740 (2002).
72. Liu, S., et al. Expression of autotaxin and lysophosphatidic acid receptors increases mammary tumorigenesis, invasion, and metastases. Cancer Cell 15, 539-550 (2009).
73. Zhang, G. Zhao, Z. Xu, S., Ni, L. & Wang, X. Expression of autotaxin mRNA in human hepatocellular carcinoma. Chin Med J (Engl) 112, 330-332 (1999).
74. Stassar, M. J., et al. Identification of human renal cell carcinoma associated genes by suppression subtractive hybridization. Br J Cancer 85, 1372-1382 (2001).
75. Kishi, Y., et al. Autotaxin Is Overexpressed in Glioblastoma Multiforme and Contributes to Cell Motility of Glioblastoma by Converting Lysophosphatidylcholine TO Lysophosphatidic Acid. J Biol Chem 281, 17492-17500 (2006).
76. Yang, Y., Mou, L., Liu, N. & Tsao. M. S. Autotaxin expression in non-small-cell lung cancer. Am J Respir Cell Mol Biol 21, 216-222 (1999).
77. Toews, M. L., Ediger, T. L., Romberger, D. J. & Rennard, S. I. Lysophosphatidic acid in airway function and disease. Biochim Biophys Acta 1582, 240-250 (2002).

The following references are numbered in accord with Examples 14-19.
1. Xu X., Yang G. Zhang H., and Prestwich G. D., Prostaglandins & Other Lipid Mediators, 2009, 89(3-4): p. 140-146.
2. Pamuklar Z. Federico L., et al., J. Biol. Chem., 2009, 284(11): p. 7385-7394.
3. Fotopoulou S., Oikonomou N., et al., Developmental Biology. 3839(2): p. 451-464.

The disclosure of all references cited herein are incorporated herein by reference. The invention has been described in detail with reference to preferred embodiments thereof. However, it will be appreciated that those skilled in the art, upon consideration of this disclosure, may make modifications and improvements within the spirit and scope of the invention.

1. A method for preventing, treating, or reducing symptoms of an inflammatory disorder, autoimmune disorder, or fibrosis or malignancy of the lung, the method comprising administering to the mammal a sufficient amount of an autotaxin (ATX) inhibitor, to prevent, treat or reduce symptoms of the inflammatory disorder, autoimmune disorder, or the fibrosis or malignancy of the lung.

2-11. (canceled)

12. A method for the diagnosis of an inflammatory disorder, autoimmune disorder, or fibrosis or malignancy of the lung in a subject, comprising obtaining a biological sample from the mammal and detecting an increase in the amount of autotaxin (ATX) or product thereof and/or a decrease in substrate levels compared to a control, wherein the increase is taken to be indicative of the presence of the inflammatory disorder, autoimmune disorder, or the fibrosis or malignancy of the lung in the subject.

13. The method of claim 12, wherein the inflammatory disorder is rheumatoid arthritis.

14. The method of claim 12, wherein the autoimmune disorder is multiple sclerosis.

15. The method of claim 12, wherein the fibrosis of the lung is an interstitial lung disease.

16. The method of claim 15, wherein the interstitial lung disease is pulmonary fibrosis.

17. The method of claim 12, wherein the method further comprises the step of contacting the sample with an antibody that specifically binds autotaxin (ATX) or an ATX binding fragment thereof and detecting the ATX as being indicative of the presence of the inflammatory disorder, autoimmune disorder, or the fibrosis or malignancy of the lung in the subject.

18. The method of claim 12, wherein the detection step further comprises contacting the biological sample with a detectable ATX substrate under conditions sufficient to produce a detectable product, wherein presence of the detectable product is taken to be indicative of the amount of ATX in the biological sample.

19. The method of claim 12, wherein the ATX product is lysophosphatidic acid (LPA) or a metabolite thereof; and the method further comprises the step of contacting the sample with an antibody that specifically binds the LPA or an LPA-binding fragment thereof; and detecting the LPA or metabolite as being indicative of the presence of the inflammatory disorder, autoimmune disorder, or the fibrosis or malignancy of the lung in the subject.

20. The method of claim 12, wherein the ATX substrate is lysophosphatidylcholine (LPC) or a precursor thereof; and the method further comprises the step of detecting the LPC or precursor by performing one or more of chromatography and mass spectrometery, and detecting the LPC or precursor as being indicative of the presence of the inflammatory disorder, autoimmune disorder, or the fibrosis or malignancy of the lung in the subject.

21. The method of claim 12, wherein the ATX product is lysophosphatidic acid (LPA) or a metabolite thereof and the method further comprises the step of detecting the LPA or metabolite by performing one or more of chromatography and mass spectrometery, and detecting the LPA or metabolite as being indicative of the presence of the inflammatory disorder, autoimmune disorder, or the fibrosis or malignancy of the lung in the subject.

22. The method of claim 12, wherein the method further comprises the step of detecting a nucleic acid that encodes the ATX.

23. The method of claim 22, wherein the method further comprises the step of performing a polymerase chain reaction (PCR) step to amplify the nucleic acid.

24. A method for preventing, treating, or reducing symptoms of sepsis or an acute lung injury comprising administering to the mammal a sufficient amount of autotaxin (ATX) or a biologically active fragment thereof.

25. The method of claim 24 wherein the acute lung injury is induced by a ventilator.

26. The method of claim 24, wherein the acute lung injury is acute respiratory distress syndrome.

27. A method for preventing, treating, or reducing symptoms of an inflammatory disorder, autoimmune disorder, the method comprising administering to the mammal a sufficient amount of an lysophohatidyl acid (LPA) signaling inhibitor, to prevent, treat or reduce symptoms of the inflammatory disorder or autoimmune disorder.

28-31. (canceled)

32. A kit for performing the method of claims 12 or 24.


SEQUENCE LISTING (23 sequences)

SEQ ID NO: 1 - DNA, 25 bp, Artificial
  primer
gtgaaatattcttaatgcctctctg

SEQ ID NO: 2 - DNA, 24 bp, Artificial
  primer
gccttccacatactgtttaattcc

SEQ ID NO: 3 - DNA, 21 bp, Artificial
  primer
ttctggtgcttgtctcactga

SEQ ID NO: 4 - DNA, 22 bp, Artificial
  primer
cagtatgttcggcttcccattc

SEQ ID NO: 5 - DNA, 20 bp, Artificial
  primer
cagatgatgggagagaagca

SEQ ID NO: 6 - DNA, 20 bp, Artificial
  primer
cggcaagtcttcagagtagt

SEQ ID NO: 7 - DNA, 14 bp, Artificial
  primer
gaggaatcgggaca

SEQ ID NO: 8 - DNA, 14 bp, Artificial
  primer
tgaaggtggcgctc

SEQ ID NO: 9 - DNA, 24 bp, Artificial
  primer
gaccacactcagcctagtcaagac

SEQ ID NO: 10 - DNA, 18 bp, Artificial
  primer
cagcatctcggcaggaat

SEQ ID NO: 11 - DNA, 24 bp, Artificial
  primer
gctcccatgaagctaatgaagaca

SEQ ID NO: 12 - DNA, 18 bp, Artificial
  primer
tacgagtagatgatgggg

SEQ ID NO: 13 - DNA, 21 bp, Artificial
  primer
agtgcctccctgtttgtcttc

SEQ ID NO: 14 - DNA, 23 bp, Artificial
  primer
gccagtggcgattaaagttgtaa

SEQ ID NO: 15 - DNA, 18 bp, Artificial
  primer
accctggaggtgaaagtc

SEQ ID NO: 16 - DNA, 18 bp, Artificial
  primer
gaccaccatatgcaaacg

SEQ ID NO: 17 - DNA, 20 bp, Artificial
  primer
cgcatttgacaggaattctt

SEQ ID NO: 18 - DNA, 20 bp, Artificial
  primer
tacacaacacagccgtctca

SEQ ID NO: 19 - DNA, 23 bp, Artificial
  primer
gaccctaaagccattattgctaa

SEQ ID NO: 20 - DNA, 20 bp, Artificial
  primer
gggaaggtgctgtttcatgt

SEQ ID NO: 21 - DNA, 20 bp, Artificial
  primer
cgcatttgacaggaattctt

SEQ ID NO: 22 - DNA, 20 bp, Artificial
  primer
tacacaacacagccgtctca

SEQ ID NO: 23 - DNA, 20 bp, Artificial
  primer
atcaaaatactggggctgcc